(19) United States
(12) Patent Application Publication
(10) Pub. No.: US 2002/0082420 A1
(43) Pub. Date: Jun. 27, 2002

(54) Bicyclic heterocycles, pharmaceutical compositions containing them, their use, and processes for preparing them

(76) Inventors: Frank Himmelsbach, Mittelbiberach (DE); Elke Langkopf, Warthausen (DE); Stefan Blech, Warthausen (DE); Birgit Jung, Schwabenheim (DE); Thomas Metz, Vienna (AT)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/933,597

(22) Filed: Aug. 21, 2001

Related U.S. Application Data
(63) Continuation of application No. PCT/EP00/02229, filed on Mar. 14, 2000.

(51) Int. Cl. C07D 487/04; C07D 239/94; C07D 239/42

(52) U.S. Cl. 544/262; 544/280; 544/293; 544/327

(57) ABSTRACT

A compound of formula (I)

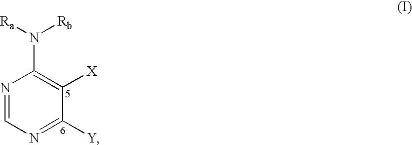

wherein Rₐ, R_{b}, X, and Y are as defined herein, or a tautomer, stereoisomer, or salt thereof, particularly a physiologically acceptable salt thereof. In addition, pharmaceutical compositions comprising an effective amount of a compound of formula (I), methods for the treatment or prophylaxis of benign or malignant tumors, diseases of the airways and lungs, polyps, diseases of the gastrointestinal tract, bile duct, gall bladder, kidneys, and skin, and methods for making compounds of formula (I) are disclosed.

### RELATED APPLICATIONS

This application is a continuation of International Application No. PCT/EP00/02229, filed on Mar. 14, 2000, benefit of which is hereby claimed, pursuant to 35 U.S.C. § 365(c) and § 120.

### SUMMARY OF THE INVENTION

The present invention relates to bicyclic heterocyclic compounds of general formula

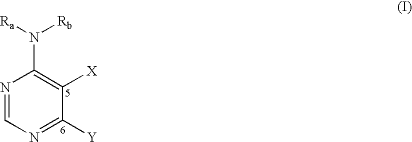

the tautomers, the stereoisomers, and the salts thereof, particularly the physiologically acceptable salts thereof with inorganic or organic acids or bases which have valuable pharmacological properties, particularly an inhibiting effect on signal transduction mediated by tyrosine kinases, their use in treating diseases, particularly tumoral diseases, diseases of the lungs and respiratory tract, and the preparation thereof.

### DETAILED DESCRIPTION OF THE INVENTION

In the bicyclic heterocyclic compounds of general formula I

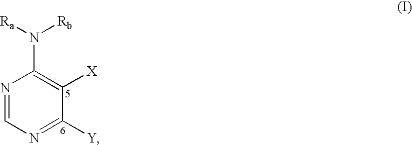

Rₐ denotes a hydrogen atom or a C₁₋₄-alkyl group,

R_{b} denotes a phenyl, benzyl, or 1-phenylethyl group wherein the phenyl nucleus is substituted in each case by the groups R₁ to R₃, wherein

R₁ and R₂, which may be identical or different, each denote a hydrogen, fluorine, chlorine, bromine, or iodine atom,

a C₁₋₄-alkyl, hydroxy, C₁₋₄-alkoxy, C₃₋₆-cycloalkyl, C₄₋₆-cycloalkoxy, C₂₋₅-alkenyl, or C₂₋₅-alkynyl group,

an aryl, aryloxy, arylmethyl, or arylmethoxy group,

a C₃₋₅-alkenyloxy or C₃₋₅-alkynyloxy group, wherein the unsaturated moiety may not be linked to the oxygen atom,

a C₁₋₄-alkylsulfenyl, C₁₋₄-alkylsulfinyl, C₁₋₄-alkylsulfonyl, C₁₋₄-alkylsulfonyloxy, trifluoromethylsulfenyl, trifluoromethylsulfinyl, or trifluoromethylsulfonyl group,

a methyl or methoxy group substituted by 1 to 3 fluorine atoms,

an ethyl or ethoxy group substituted by 1 to 5 fluorine atoms,

a cyano or nitro group or an amino group optionally substituted by one or two C₁₋₄-alkyl groups, wherein the substituents may be identical or different,

or R₁ together with R₂, if they are bound to adjacent carbon atoms, denote a —CH═CH—CH═CH, —CH═CH—NH, or —CH═N—NH group, and

R₃ denotes a hydrogen, fluorine, chlorine, or bromine atom, a C1-4-alkyl, trifluoromethyl, or C₁₋₄-alkoxy group,

X and Y together denote a —N═C(—A—B)—CH═CH—, —CH═N—C(—A—B)═CH—, —CH═C(—A—B)—N═CH—, —CH═CH—C(—A—B)=N—, —N═C(—A—B)—N═CH—, or —CH═N—C(—A—B)═N bridge, wherein the left-hand end of these bridges is linked to position 5 and the right-hand end of these bridges is linked to position 6 of the pyrimidine ring,

A denotes an —O—C₁₋₈-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom of the abovementioned group in each case is linked to the bicyclic heteroaromatic compound,

an —NR₄—C₁₋₈-alkylene, —NR₄—C₃₋₇-cycloalkylene, —NR₄-C₁₋₃-alkylene-C₃₋₇-cycloalkylene, —NR₄-C₃₋₇-cycloalkylene-C₁₋₃-alkylene, or —NR₄—C₁₋₃-alkylene-C₃₋₇-cycloalkylene-C₁₋₃-alkylene group, wherein the —NR₄— moiety of the abovementioned groups in each case is linked to the bicyclic heteroaromatic compound, and

R₄ denotes a hydrogen atom or a C₁₋₄-alkyl group,

an oxygen atom which is linked to a carbon atom of the group B, an —NR₄—C₄₋₇-cycloalkylene-NH—SO₂—C₁₋₄-alkylene or —NR₄—C₄₋₇-cycloalkylene-N(C₁₋₄-alkyl)-SO₂—C₁₋₄-alkylene group, wherein the —NR₄— moiety of the abovementioned groups in each case is linked to the bicyclic heteroaromatic compound and R₄ is as hereinbefore defined,

an —NR₄ group, where the latter is linked to a carbon atom of the group B and R₄ is as hereinbefore defined,

an azetidinylene, pyrrolidinylene, piperidinylene, or hexahydroazepinylene group optionally substituted by one or two methyl groups, wherein in each case the cyclic nitrogen atom of the abovementioned groups is linked to the bicyclic heteroaromatic compound,

an azetidinylene-C₁₋₃-alkylene, pyrrolidinylene-C₁₋₃-alkylene, piperidinylene-C₁₋₃-alkylene, or hexahydroazepinylene-C₁₋₃-alkylene group, wherein in each case the cyclic nitrogen atom of the abovementioned groups is linked to the bicyclic heteroaromatic compound,

a 1,4-piperazinylene or 1,4-homopiperazinylene group, these groups each being linked to a carbon atom of the group B,

a 1,4-piperazinylene-C₁₋₃-alkylene or 1,4-homopiperazinylene-C₁₋₃-alkylene group, wherein in each case the cyclic nitrogen atom of the abovementioned groups is linked to the bicyclic heteroaromatic compound, an —NR₄-azetidinylene, —NR₄-pyrrolidinylene, —NR₄-piperidinylene, or —NR₄-hexahydroazepinylene group, wherein the —NR₄— moiety of the abovementioned groups is linked in each case to the bicyclic heteroaromatic compound and in each case the cyclic nitrogen atom of the abovementioned groups is linked to a carbon atom of the group B,

an —NR₄-azetidinylene-C₁₋₃-alkylene, —NR₄-pyrrolidinylene-C₁₋₃-alkylene, —NR₄-piperidinylene-C₁₋₃-alkylene, or —NR₄-hexahydroazepinylene-C₁₋₃-alkylene group, wherein in each case the —NR₄— moiety of the abovementioned groups is linked to the bicyclic heteroaromatic compound and the cyclic nitrogen atom of the abovementioned groups is in each case linked to the alkylene moiety,

an —NR₄—C₃₋₇-cycloalkylenecarbonyl group, wherein the —NR₄— moiety is linked to the bicyclic heteroaromatic compound and the carbonyl group is linked to a nitrogen atom of the group B,

an —NR₄—C₃₋₇-cycloalkylenecarbonylamino group, wherein the —NR₄— moiety is linked to the bicyclic heteroaromatic compound and the nitrogen atom of the carbonylamino moiety, which may additionally be substituted by a C₁₋₄-alkyl group, is linked to a carbon atom of the group B,

an —NR₄-C₃₋₇-cycloalkylenecarbonylamino-C₁₋₃-alkylene group, wherein the —NR₄— moiety is linked to the bicyclic heteroaromatic compound and the nitrogen atom of the carbonylamino moiety may additionally be substituted by a C₁₋₄-alkyl group,

an azetidinylenecarbonyl, pyrrolidinylenecarbonyl, piperidinylenecarbonyl, or hexahydroazepinylenecarbonyl group, wherein in each case the cyclic nitrogen atom of the abovementioned groups is linked to the bicyclic heteroaromatic compound and the carbonyl group in each case is linked to a nitrogen atom of the group B,

an azetidinylenecarbonylamino, pyrrolidinylenecarbonylamino, piperidinylenecarbonylamino, or hexahydroazepinylenecarbonylamino group, wherein in each case the cyclic nitrogen atom of the abovementioned groups is linked to the bicyclic heteroaromatic compound and the nitrogen atom of the carbonylamino moiety, which may additionally be substituted by a C₁₋₄-alkyl group, is linked to a carbon atom of the group B,

an azetidinylenecarbonylamino-C₁₋₃-alkylene, pyrrolidinylenecarbonylamino-C₁₋₃-alkylene, piperidinylenecarbonylamino-C₁₋₃-alkylene, or hexahydroazepinylenecarbonylamino-C₁₋₃-alkylene group, wherein in each case the cyclic nitrogen atom of the abovementioned groups is linked to the bicyclic heteroaromatic compound and the nitrogen atom of the carbonylamino moiety may additionally be substituted by a C₁₋₄-alkyl group, and

B denotes an R₆O—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group, wherein

R₅ denotes a hydrogen atom,

a C₁₋₄-alkyl group, which may be substituted by a hydroxy, C₁₋₄-alkoxy, carboxy, R₆O—CO, (R₇O—PO—OR₈), (R₇O—PO—R₉), amino, C₁₋₄-alkylamino, or di-(C₁₋₄-alkyl)-amino group, or by a 4- to 7-membered alkyleneimino group, wherein in the abovementioned 6- to 7-membered alkyleneimino groups in each case a methylene group in the 4 position may be replaced by an oxygen or sulfur atom, by a sulfinyl, sulfonyl, imino, or N—(C₁₋₄-alkyl)-imino group,

a C₃₋₇-cycloalkyl or C₃₋₇-cycloalkyl-C₁₋₃-alkyl group,

R₆, R₇, and R₈, which may be identical or different, in each case denote a hydrogen atom,

a C₁₋₈-alkyl group which may be substituted by a hydroxy, C₁₋₄-alkoxy, amino, C₁₋₄-alkylamino, or di-(C₁₋₄-alkyl)-amino group, or by a 4- to 7-membered alkyleneimino group, wherein in the abovementioned 6- to 7-membered alkyleneimino groups in each case a methylene group in the 4 position may be replaced by an oxygen or sulfur atom, by a sulfinyl, sulfonyl, imino, or N—(C₁₋₄-alkyl)-imino group,

a C₄₋₇-cycloalkyl group optionally substituted by one or two methyl groups,

a C₃₋₅-alkenyl or C₃₋₅-alkynyl group, wherein the unsaturated moiety may not be linked to the oxygen atom,

a C₃₋₇-cycloalkyl-C₁₋₄-alkyl, aryl, aryl-C₁₋₄-alkyl, or RₑCO—O—(R_{c}CR_{d}) group, wherein

R_{c} and R_{d}, which may be identical or different, in each case denote a hydrogen atom or a C₁₋₄-alkyl group, and

Rₑ denotes a C₁₋₄-alkyl, C₃₋₇-cycloalkyl, C₁₋₄-alkoxy, or C₅₋₇-cycloalkoxy group,

and R₉ denotes a C₁₋₄-alkyl, aryl, or aryl-C₁₋₄-alkyl group, a 4- to 7-membered alkyleneimino group which is substituted by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a piperazino or homopiperazino group which is substituted in the 4 position by the group R₁₀ and additionally at a cyclic carbon atom by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined, and

R₁₀ denotes a hydrogen atom, a C₁₋₄-alkyl, formyl, C₁₋₄-alkylcarbonyl, or C₁₋₄-alkylsulfonyl group,

a piperazino or homopiperazino group which is substituted in each case in the 4 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a pyrrolidinyl, piperidinyl, or hexahydroazepinyl group substituted in the 1 position by the group R₁₀, wherein the abovementioned 5- to 7-membered rings are each additionally substituted at a carbon atom by an R₆O—CO, (R₇O—PO—OR₉), (R₇O—PO—R₉), R₆O—CO—C₁₋₄-alkyl, bis(R₆O—CO)—C₁₋₄-alkyl, (R₇O—PO—OR₈)-C1-4-alkyl, or (R₇O—PO—R₉)-C₁₋₄-alkyl group wherein R₆ to R₁₀ are as hereinbefore defined,

a pyrrolidinyl, piperidinyl, or hexahydroazepinyl group substituted in the 1 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a 2-oxomorpholino group, which may be substituted by one or two methyl groups,

a 2-oxomorpholinyl group which is substituted in the 4 position by a hydrogen atom, by a C₁₋₄-alkyl, R₆O—CO—C₁₋₄-alkyl, (R₇O—PO—OR₈)-C₁₋₄-alkyl, or (R₇O—PO—R₉)—C₁₋₄-alkyl group, wherein R₆ to R₉ are as hereinbefore defined and the abovementioned 2-oxomorpholinyl groups are in each case linked to a carbon atom of the group A,

a C₅₋₇-cycloalkyl group which is substituted by an amino, C₁₋₄-alkylamino, or di-(C₁₋₄-alkyl)-amino group and by an R₆O—CO group, wherein R₆ is as hereinbefore defined,

a C₅₋₇-cycloalkyl group wherein a methylene group is replaced by an R₆—CO—C₁₋₄-alkyleneimino, [bis(R₆—CO)-C₁₋₄-alkylene]imino, (R₇O—PO—OR₈)-C₁₋₄-alkyleneimino, or (R₇O—PO—R₉)-C₁₋₄-alkyleneimino group and in each case two hydrogen atoms in the cycloalkyl moiety are replaced by a straight-chained alkylene bridge, this bridge containing 2 to 6 carbon atoms, if the two hydrogen atoms are located at the same carbon atom, or contains 1 to 5 carbon atoms if the two hydrogen atoms are located at adjacent carbon atoms, or contains 2 to 4 carbon atoms, if the two hydrogen atoms are located at carbon atoms which are separated by one atom, wherein R₆ to R₉ are as hereinbefore defined,

or A together with B denotes a 1-azetidinyl group wherein the two hydrogen atoms of a methylene group are replaced by a straight-chained C₄₋₆-alkylene bridge, wherein in each case a methylene group in the C₄₋₆-alkylene bridge is replaced by an R₆—CO—C₁₋₄-alkyleneimino, [bis(R₆—CO)—C₁₋₄-alkylene]imino, (R₇O—PO—OR₉)—C₁₋₄-alkyleneimino, or (R₇O—PO—R₉)—C₁₋₄-alkyleneimino group wherein R₆ to R₉ are as hereinbefore defined,

a 1-pyrrolidinyl, 1-piperidinyl, or 1-azacyclohept-1-yl group wherein the two hydrogen atoms of a methylene group are replaced by a straight-chained C₃₋₆-alkylene bridge, wherein in each case a methylene group in the C₃₋₆-alkylene bridge is replaced by an R₆—CO—C₁₋₄-alkyleneimino, [bis(R₆—CO)—C₁₋₄-alkylene]imino, (R₇O—PO—OR₈)—C₁₋₄-alkyleneimino, or (R₇O—PO—R₉)—C₁₋₄-alkyleneimino group wherein R₆ to R₉ are as hereinbefore defined,

a pyrrolidino, piperidino, or hexahydroazepino group which are substituted in each case by an amino, C₁₋₄-alkylamino, or di-(C₁₋₄-alkyl)-amino group and by an R₆—CO group, wherein R₆ is as hereinbefore defined,

a piperazino or homopiperazino group which is substituted in the 4 position by the group R₁₀ and additionally at a cyclic carbon atom by an R₆—CO, (R₇O—PO—OR₈), (R₇O—PO—R₉), R₆—CO—C₁₋₄-alkyl, bis(R₆—CO)-C₁₋₄-alkyl, (R₇O—PO—OR₈)—C₁₋₄-alkyl, or (R₇O—PO—R₉)—C₁₋₄-alkyl group wherein R₆ to R₁₀ are as hereinbefore defined,

a piperazino or homopiperazino group which is substituted in each case in the 4 position by an R₆—CO—C₁₋₄-alkyl, bis(R₆—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined, or

a 2-oxomorpholino group, which may be substituted by one or two methyl groups,

wherein by the aryl moieties mentioned in the definition of the abovementioned groups is meant a phenyl group which may in each case be monosubstituted by R₁₁, mono-, di-, or trisubstituted by R₁₂ or monosubstituted by R₁₁ and additionally mono- or disubstituted by R₁₂, wherein the substituents may be identical or different, and

R₁₁ may denote a cyano, carboxy, C₁₋₄-alkoxycarbonyl, aminocarbonyl, C₁₋₄-alkylaminocarbonyl, di-(C₁₋₄-alkyl)-aminocarbonyl, C₁₋₄-alkylsulfenyl, C₁₋₄-alkylsulfinyl, C₁₋₄-alkylsulfonyl, hydroxy, C₁₋₄-alkylsulfonyloxy, trifluoromethyloxy, nitro, amino, C₁₋₄-alkylamino, di-(C₁₋₄-alkyl)-amino, C₁₋₄-alkylcarbonylamino, N-(C₁₋₄-alkyl)-C₁₋₄-alkylcarbonylamino, C₁₋₄-alkylsulfonylamino, N—(C₁₋₄-alkyl)-C₁₋₄-alkylsulfonylamino, aminosulfonyl, C₁₋₄-alkylaminosulfonyl, or di-(C₁₋₄-alkyl)-aminosulfonyl group or a carbonyl group which is substituted by a 5- to 7-membered alkyleneimino group, wherein in the abovementioned 6- to 7-membered alkyleneimino groups in each case a methylene group in the 4 position may be replaced by an oxygen or sulfur atom, by a sulfinyl, sulfonyl, imino, or N—(C₁₋₄-alkyl)-imino group, and

R₁₂ denotes a fluorine, chlorine, bromine, or iodine atom, a C₁₋₄-alkyl, trifluoromethyl, or C₁₋₄-alkoxy group or

two R₁₂ groups, if they are bound to adjacent carbon atoms, together denote a C₃₋₅-alkylene, methylenedioxy, or 1,3-butadien-1,4-ylene group.

Preferred compounds of the above general formula I are those wherein Rₐ, R_{b}, X, and Y are as hereinbefore defined, with the proviso that A does not denote an —NR₄—C₄₋₇-cycloalkylene-NH—SO₂-C₁₋₄-alkylene or —NR₄-C₄₋₇-cycloalkylene-N(C₁₋₄-alkyl)-SO₂—C₁₋₄-alkylene group, wherein the —NR₄— moiety of the abovementioned groups in each case is linked to the bicyclic heteroaromatic compound and R₄ is as hereinbefore defined, and

does not denote an azetidinylene, pyrrolidinylene, piperidinylene, or hexahydroazepinylene group substituted by one or two methyl groups, wherein in each case the cyclic nitrogen atom of the abovementioned groups is linked to the bicyclic heteroaromatic compound,

the tautomers, stereoisomers, and salts thereof.

Particularly preferred compounds of general formula I are those wherein

Rₐ denotes a hydrogen atom,

R_{b} denotes a phenyl, benzyl, or 1-phenylethyl group wherein the phenyl nucleus is substituted in each case by the groups R₁ to R₃, wherein

R₁ and R₂, which may be identical or different, each denote a hydrogen, fluorine, chlorine, or bromine atom,

a methyl, trifluoromethyl, ethynyl, or amino group,

a phenyl, phenoxy, benzyl, or benzyloxy group or R₁ together with R₂, if they are bound to adjacent carbon atoms, denote a —CH═CH—NH or —CH═N—NH group, and

R₃ denotes a hydrogen, fluorine, chlorine, or bromine atom,

X and Y together denote a —N═C(—A—B)—CH═CH—, —CH═N—C(—A—B)═CH—, —CH═C(—A—B)—N═CH—, —CH═CH—C(—A—B)═N—, —N═C(—A—B)—N═CH—, or —CH═N—C(—A—B)═N— bridge, wherein

the left-hand end of these bridges is linked to position 5 and the right-hand end of these bridges is linked to position 6 of the pyrimidine ring,

A denotes an —NR₄—C₁₋₄-alkylene, —NR₄-cyclohexylene, —NR₄-cyclohexylene-NH—SO₂—C₁₋₃-alkylene, —NR₄-C₁₋₃-alkylene-cyclohexylene, —NR₄-cyclohexylene-C₁₋₃-alkylene, or —NR₄-C₁₋₃-alkylene-cyclohexylene-C₁₋₃-alkylene group, wherein the —NR₄— moiety of the abovementioned groups in each case is linked to the bicyclic heteroaromatic compound, and

R₄ denotes a hydrogen atom or a methyl group,

an —NR₄ group, the latter being linked to a carbon atom of the group B and R₄ is as hereinbefore defined,

a pyrrolidinylene or piperidinylene group optionally substituted by one or two methyl groups, wherein in each case the cyclic nitrogen atom of the abovementioned groups is linked to the bicyclic heteroaromatic compound,

a piperidinylene-C₁₋₃-alkylene group, wherein the cyclic nitrogen atom of the abovementioned group is linked to the bicyclic heteroaromatic compound,

a 1,4-piperazinylene or 1,4-homopiperazinylene group, these groups each being linked to a carbon atom of the group B,

a 1,4-piperazinylene-C₁₋₂-alkylene or 1,4-homopiperazinylene-C₁₋₂-alkylene group, wherein in each case the cyclic nitrogen atom of the abovementioned groups is linked to the bicyclic heteroaromatic compound,

an —NR₄-piperidinylene group, wherein the —NR₄— moiety of the abovementioned group is linked to the bicyclic heteroaromatic compound and the cyclic nitrogen atom of the abovementioned group is linked to a carbon atom of the group B,

an —NR₄-piperidinylene-C₁₋₂-alkylene group, wherein the —NR₄— moiety of the abovementioned group is linked to the bicyclic heteroaromatic compound and the cyclic nitrogen atom of the abovementioned group is linked to the alkylene moiety,

an —NR₄-cyclohexylenecarbonyl group, wherein the —NR₄— moiety is linked to the bicyclic heteroaromatic compound and the carbonyl group is linked to a nitrogen atom of the group B,

an —NR₄-cyclohexylenecarbonylamino group, wherein the —NR₄— moiety is linked to the bicyclic heteroaromatic compound and the nitrogen atom of the carbonylamino moiety is linked to a carbon atom of the group B,

an —NR₄-cyclohexylenecarbonylamino-C₁₋₂-alkylene group, wherein the —NR₄- moiety is linked to the bicyclic heteroaromatic compound,

a piperidinylenecarbonyl group, wherein the cyclic nitrogen atom of the abovementioned group is linked to the bicyclic heteroaromatic compound and the carbonyl group is linked to a nitrogen atom of the group B,

a piperidinylenecarbonylamino group, wherein the cyclic nitrogen atom of the abovementioned group is linked to the bicyclic heteroaromatic compound and the nitrogen atom of the carbonylamino moiety is linked to a carbon atom of the group B,

a piperidinylenecarbonylamino-C₁₋₂-alkylene group, wherein the cyclic nitrogen atom of the abovementioned group is linked to the bicyclic heteroaromatic compound, and B denotes an R₆—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, may additionally be substituted by one or two C₁₋₂-alkyl groups or by an R₆—CO or R₆—CO—C₁₋₂-alkyl group, wherein

R₅ denotes a hydrogen atom or a C₁₋₄-alkyl group which may be substituted by an R₆—CO group, R₆, R₇, and R₈, which may be identical or different, in each case denote a hydrogen atom,

a C₁₋₈-alkyl group,

a cyclopentyl, cyclopentylmethyl, cyclohexyl, or cyclohexylmethyl group,

a phenyl group optionally substituted by one or two methyl groups, a 5-indanyl group or a benzyl group optionally substituted in the phenyl moiety by one or two methyl groups, and

R₉ denotes a methyl or ethyl group,

a pyrrolidino or piperidino group which is substituted in each case by an R₆O—CO or R₆O—CO—C₁₋₄-alkyl group wherein R₆ is as hereinbefore defined,

a piperazino or homopiperazino group which is substituted in each case in the 4 position by the group R₁₀ and is additionally substituted at a cyclic carbon atom by an R₆O—CO or R₆O—CO—C₁₋₄-alkyl group wherein R₆ is as hereinbefore defined, and

R₁₀ denotes a hydrogen atom or a methyl or ethyl group,

a piperazino or homopiperazino group which is substituted in each case in the 4 position by an R₆—CO—C₁₋₄-alkyl, bis(R₆—CO)-C₁₋₄-alkyl, (R₇O—PO—OR₈)-C₁₋₄-alkyl, or (R₇O—PO-R₉)-C₁₋₄-alkyl group wherein R₆ to R₉ are as hereinbefore defined,

a pyrrolidinyl or piperidinyl group substituted in the 1 position by the group R₁₀, which is additionally substituted in each case at a carbon atom by an R₆O—CO or R₆—CO—C₁₋₄-alkyl group wherein R₆ is as hereinbefore defined,

a pyrrolidinyl, piperidinyl, or hexahydroazepinyl group substituted in the 1 position by an RrO—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a 2-oxomorpholino group, which may be substituted by one or two methyl groups,

a 2-oxomorpholinyl group which is substituted in the 4 position by a hydrogen atom, by a methyl, ethyl, or R₆O—CO—C₁₋₄-alkyl group, wherein R₆ is as hereinbefore defined and the abovementioned 2-oxomorpholinyl groups in each case are linked to a carbon atom of the group A,

a C₅₋₆-cycloalkyl group which is substituted by an amino, C₁₋₂-alkylamino, or di-(C₁₋₂-alkyl)-amino group and by an R₆O—CO group, wherein R₆ is as hereinbefore defined,

or A and B together denote a 1-pyrrolidinyl or 1-piperidinyl group wherein the two hydrogen atoms of a methylene group are replaced by a straight-chained C₄₋₅-alkylene bridge, wherein in each case a methylene group in the C₄₋₅-alkylene bridge is replaced by an R₆O—CO—C₁₋₄-alkyleneimino group wherein R₆ is as hereinbefore defined,

a pyrrolidino or piperidino group which is substituted in each case by an amino, C₁₋₂-alkylamino, or di-(C₁₋₂-alkyl)-amino group and by an R₆O—CO group, wherein R₆ is as hereinbefore defined,

a piperazino or homopiperazino group which is substituted in the 4 position by the group R₁₀ and additionally at a cyclic carbon atom by an R₆O—CO or R₆O—CO—C₁₋₄-alkyl group wherein R₆ and RIO are as hereinbefore defined,

a piperazino or homopiperazino group which is substituted in each case in the 4 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined, or

a 2-oxomorpholino group, which may be substituted by one or two methyl groups,

particularly those compounds of general formula I wherein

Rₐ denotes a hydrogen atom,

R_{b} denotes a phenyl, benzyl, or 1-phenylethyl group wherein the phenyl nucleus is substituted in each case by the groups R₁ to R₃, wherein

R₁ and R₂, which may be identical or different, each denote a hydrogen, fluorine, chlorine, or bromine atom,

a methyl, trifluoromethyl, ethynyl, or amino group

or R₁ together with R₂, if they are bound to adjacent carbon atoms, denote a —CH═CH—NH group, and

R₃ denotes a hydrogen, fluorine, chlorine, or bromine atom,

X and Y together denote a —N═C(—A—B)—CH═CH—, —CH═N—C(—A—B)═CH—, —CH═C(—A—B)—N═CH—, —CH═CH—C(—A—B)═N—, —N═C(—A—B)—N═CH—, or —CH═N—C(—A—B)═N— bridge, wherein the left-hand end of these bridges is linked to position 5 and the right-hand end of these bridges is linked to position 6 of the pyrimidine ring,

A denotes an —NR₄—C₁₋₄-alkylene, —NR₄-cyclohexylene, —NR₄-cyclohexylene-NH—SO₂—C₁₋₃-alkylene, —NR₄-methylene-cyclohexylene, —NR₄-cyclohexylene-methylene, or —NR₄-methylene-cyclohexylene-methylene group, wherein the —NR₄- moiety of the abovementioned groups in each case is linked to the bicyclic heteroaromatic compound, and

R₄denotes a hydrogen atom or a methyl group,

an —NR₄ group, the latter being linked to a carbon atom of the group B and R₄ is as hereinbefore defined,

a pyrrolidinylene or piperidinylene group optionally substituted by one or two methyl groups, wherein in each case the cyclic nitrogen atom of the abovementioned groups is linked to the bicyclic heteroaromatic compound,

a piperidinylene-C₁₋₂-alkylene group, wherein the cyclic nitrogen atom of the abovementioned group is linked to the bicyclic heteroaromatic compound,

a 1,4-piperazinylene group, this group being linked in each case to a carbon atom of the group B,

a 1,4-piperazinylene-C₁₋₂-alkylene group, the cyclic nitrogen atom of the abovementioned group being linked to the bicyclic heteroaromatic compound,

an —NR₄-piperidinylene group, wherein the —NR₄— moiety of the abovementioned group is linked to the bicyclic heteroaromatic compound and the cyclic nitrogen atom of the abovementioned group is linked to a carbon atom of the group B,

an —NR₄-cyclohexylenecarbonylamino group, wherein the —NR₄— moiety is linked to the bicyclic heteroaromatic compound and the nitrogen atom of the carbonylamino moiety is linked to a carbon atom of the group B,

an —NR₄-cyclohexylenecarbonylamino-C₁₋₂-alkylene group, wherein the —NR₄— moiety is linked to the bicyclic heteroaromatic compound, and

B denotes an R₆—CO-alkylene-NR₅, (R₇O—PO—OR₈)-alkylene-NR₅, or (R₇O—PO—R₉)-alkylene-NR₅ group wherein in each case the alkylene moiety is straight-chained and contains 1 to 4 carbon atoms, wherein

R₅ denotes a hydrogen atom,

a C₁₋₂-alkyl group which may be substituted by an R₆—CO group, R₆, R₇, and R₈, which may be identical or different, in each case denote a hydrogen atom,

a C₁₋₈-alkyl group,

a cyclopentyl, cyclohexyl, cyclopentylmethyl, or cyclohexylmethyl group,

a phenyl group optionally substituted by one or two methyl groups, a 5-indanyl group or a benzyl group optionally substituted in the phenyl moiety by one or two methyl groups, and

R₉ denotes a methyl or ethyl group,

a pyrrolidino or piperidino group which is substituted in each case by an R₆—CO or RrO—CO—C₁₋₂-alkyl group wherein R₆ is as hereinbefore defined,

a piperazino group which is substituted in the 4 position by an R₆—CO—C₁₋₃-alkyl, (R₇O—PO—OR₈)—C₁₃-alkyl, or (R₇O—PO—R₉)—C₁₃-alkyl group wherein R₆ to R₉ are as hereinbefore defined, with the proviso that R₈ and R₉ in each case denote a methyl or ethyl group, and

a pyrrolidinyl, piperidinyl, or hexahydroazepinyl group substituted in the 1 position by an R₆—CO—C₁₋₄-alkyl, bis(R₆—CO)—C₁₋₄-alkyl, (R₇O—PO—OR₈)—C₁₋₄-alkyl, or (R₇O—PO—R₉)—C₁₋₄-alkyl group wherein R₆ to R₉ are as hereinbefore defined,

a 2-oxomorpholino group, which may be substituted by one or two methyl groups,

or A and B together denote a 1-pyrrolidinyl or 1-piperidinyl group wherein the two hydrogen atoms of a methylene group are replaced by a straight-chained C₄₋₅-alkylene bridge, wherein in each case a methylene group in the C₄₋₅-alkylene bridge is replaced by an R₆—CO—C₁₋₂-alkyleneimino group wherein R₆ is as hereinbefore defined,

a piperidino group which is substituted by an amino group and by an R₆—CO group, wherein R₆ is as hereinbefore defined,

a piperazino group which is substituted in the 4 position by an R₆—CO—C₁₋₄-alkyl group wherein R₆ is as hereinbefore defined, or

a 2-oxomorpholino group, which may be substituted by one or two methyl groups,

the tautomers, stereoisomers, and salts thereof.

Most particularly preferred compounds of the abovementioned general formula I are those wherein X and Y together denote an —N═C(—A—B)—N═CH— bridge,

Particularly those compounds wherein

Rₐ denotes a hydrogen atom,

R_{b} denotes a phenyl, benzyl, or 1-phenylethyl group wherein the phenyl nucleus is substituted in each case by the groups R₁ to R₃, wherein

R₁ and R₂, which may be identical or different, each denote a hydrogen, fluorine, chlorine, or bromine atom,

a methyl or amino group,

or R₁ together with R₂, if they are bound to adjacent carbon atoms, denote an —CH═CH—NH group, and

R₃ denotes a hydrogen, fluorine, chlorine, or bromine atom,

X and Y together denote an —N═C(—A—B)—N═CH— bridge, wherein the left-hand end of this bridge is linked to position 5 and the right-hand end of this bridge is linked to position 6 of the pyrimidine ring,

A denotes an —NR₄—C₁₋₃-alkylene, —NR₄-cyclohexylene, or —NR₄-cyclohexylene-NH—SO₂-ethylene group, wherein the —NR₄— moiety of the abovementioned groups in each case is linked to the bicyclic heteroaromatic compound, and

R₄ denotes a hydrogen atom or a methyl group,

an —NR₄ group, the latter being linked to a carbon atom of the group B and R₄ being as hereinbefore defined,

an optionally methyl-substituted pyrrolidinylene or piperidinylene group, wherein in each case the cyclic nitrogen atom of the abovementioned groups is linked to the bicyclic heteroaromatic compound,

a piperidinylenemethylene group, wherein the cyclic nitrogen atom is linked to the bicyclic heteroaromatic compound,

a 1,4-piperazinylene group, this group being linked to a carbon atom of the group B, and

B denotes an R₆O—CO-alkylene-NR₅ group wherein the alkylene moiety is straight-chained and contains 1 to 4 carbon atoms, wherein

R₅ denotes a hydrogen atom,

a C₁₋₂-alkyl group which may be substituted by an R₆—CO group,

R₆ denotes a hydrogen atom,

a C₁₋₄-alkyl group or a cyclohexyl group,

a pyrrolidino or piperidino group which is substituted in each case by an R₆—CO or R₆—CO—C₁₋₂-alkyl group, wherein R₆ is as hereinbefore defined,

a piperazino group which is substituted in the 4 position by an R₆O—CO-methyl or (R₇O—PO—OR₈)-methyl group wherein R₆ is as hereinbefore defined, and

R₇ and R₈ in each case denotes a methyl or ethyl group,

a piperidinyl group substituted in the 1 position by an R₆—CO—C₁₋₄-alkyl, bis(R₆—CO)—C₁₋₄-alkyl, (R₇O—PO—OR₈)-methyl, or (R₇O—PO-R₉)-methyl group wherein, to R are as hereinbefore defined, and

R₉ denotes a methyl or ethyl group,

a 2-oxomorpholino group,

or A and B together denote a piperidino group which is substituted by an amino group and by an R₆O—CO group, wherein R₆ is as hereinbefore defined,

a piperazino group which is substituted in the 4 position by an R₆—CO—C₁₋₂-alkyl group, wherein R₆ is as hereinbefore defined,

the tautomers, stereoisomers, and salts thereof.

The following particularly preferred compounds of general formula I are mentioned by way of example:

(1) 4-[(3-chloro-4-fluorophenyl)amino]-6-({1-[(methoxycarbonyl)methyl]piperidin-4-yl}amino)pyrimido[5,4-d]pyrimidine,

(2) 4-[(3-chloro-4-fluorophenyl)amino]-6-[(trans-4-{N-[(methoxycarbonyl)methyl]-N-methylamino}cyclohex-1-yl)amino]pyrimido[5,4-d]pyrimidine,

(3) 4-[(3-chloro-4-fluorophenyl)amino]-6-[4-({N-[(methoxycarbonyl)methyl]-N-methylamino}methyl)piperidin-1-yl]pyrimido[5,4-d]pyrimidine

(4) 4-[(3-bromophenyl)amino]-6-({1-[(methoxycarbonyl)methyl]piperidin-4-yl}amino)pyrimido[5 ,4-d]pyrimidine,

(5) 4-[(3-chlorophenyl)amino]-6-({1-[(methoxycarbonyl)methyl]piperidin-4-yl}amino)pyrimido[5,4-d]pyrimidine

(6) 4-[(3-methylphenyl)amino]-6-({1-[(methoxycarbonyl)methyl]piperidin-4-yl}amino)pyrimido[5,4-d]pyrimidine,

(7) 4-[(4-amino-3 ,5-dichlorophenyl)amino]-6-({1-[(methoxycarbonyl)methyl]piperidin-4-yl}amino)pyrimido[5,4-d]pyrimidine,

(8) 4-[(4-amino-3,5-dibromophenyl)amino]-6-({1-[(methoxycarbonyl)methyl]piperidin-4-yl}amino)pyrimido [5,4-d]pyrimidine,

(9) 4-[(indol-5-yl)amino]-6-({1-[(methoxycarbonyl)methyl]piperidin-4-yl}amino)pyrimido[5,4-d]pyrimidine,

(10) 4-[(3-chloro-4-fluorophenyl)amino]-6-[(trans-4-{N,N-bis-[(ethoxycarbonyl)methyl]amino}cyclohex-1-yl)amino]pyrimido[5,4-d]pyrimidine,

(11) 4-[(3-chloro-4-fluorophenyl)amino]-6-[(trans-4-{N,N-bis-[(methoxycarbonyl)methyl]amino}cyclohex-1-yl)amino]pyrimido[5,4-d]pyrimidine,

(12) 4-[(3-chloro-4-fluorophenyl)amino]-6-[(trans-4-{[(methoxy-carbonyl)methyl]amino}cyclohex-1-yl)amino]pyrimido[5,4-d]pyrimidine,

(13) 4-[(3-chloro-4-fluorophenyl)amino]-6-[N-(trans-4-{N,N,-bis[(methoxycarbonyl)methyl]amino}cyclohex-1-yl)-N-methylamino]pyrimido[5,4-d]pyrimidine,

(14) 4-[(3-bromophenyl)amino]-6-(1-[1,1-bis(methoxycarbonyl)methyl]piperidin-4-yl}amino)pyrimido[5,4-d]pyrimidine,

(15) 4-[(3-bromophenyl)amino]-6-({1-[(methoxycarbonyl)methyl]piperidin-3-yl}amino)pyrimido[5,4-d]pyrimidine,

(16) 4-[(3-chloro-4-fluorophenyl)amino]-6-(1-[1,1-bis(methoxycarbonyl)methyl]piperidin-4-yl}amino)pyrimido[5,4-d]pyrimidine,

(17) 4-[(3-bromophenyl)amino]-6-({1-[(diethoxyphosphoryl)methyl]piperidin-4-yl}amino)pyrimido[5,4-d]pyrimidine,

(18) 4-[(3-bromophenyl)amino]-6-[(1-{[(ethoxy)(methyl)phosphoryl]methyl}piperidin-4-yl)amino]pyrimido[5,4-d]pyrimidine,

(19) 4-[(3-chloro-4-fluorophenyl)amino]-6-{[trans-4-(2-oxomorpholin-4-yl)cyclohex-1-yl]amino }pyrimido[5 ,4-d]pyrimidine

and the salts thereof.

The compounds of general formula I may be prepared, for example, by the following methods:

a) reacting a compound of general formula

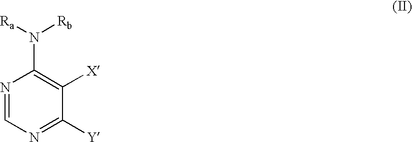

wherein

Rₐ and R_{b} are as hereinbefore defined,

X′ and Y′ together denote a —N═CZ₁—CH═CH—, —CH═N—CZ₁═CH—, —CH═CZ₁—N═CH—, —CH═CH—CZ₁═N—, —N═CZ₁—N═CH—, or —CH═N—CZ₁═N— bridge wherein Z₁ denotes an exchangeable group such as a halogen atom or a substituted sulfinyl or sulfonyl group, e.g., a chlorine or bromine atom, a methylsulfinyl, propylsulfinyl, phenylsulfinyl, benzylsulfinyl, methylsulfonyl, propylsulfonyl, phenylsulfonyl, or benzylsulfonyl group, with a compound of general formula

H—A—B (III)

wherein

A and B are as hereinbefore defined.

The reaction is optionally carried out in a solvent or mixture of solvents such as methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine, pyridine, or 2-dimethylaminopyridine, in the presence of N-ethyl-diisopropylamine (Hünig's base), wherein these organic bases may simultaneously serve as solvents, or in the presence of an inorganic base such as sodium carbonate, potassium carbonate, or sodium hydroxide solution conveniently at temperatures between −20° C. and 200° C., preferably at temperatures between 0° C. and 150° C.

b) In order to prepare a compound of general formula I wherein at least one of the groups R₆ to R₈ denote a hydrogen atom:

Converting a compound of general formula

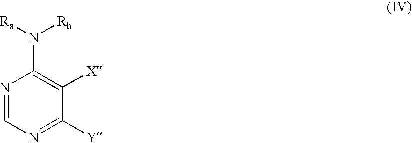

wherein

Rₐ and R_{b} are as hereinbefore defined,

X″ and Y″ together denote a —N═C(—A—B′)—CH═CH—, —CH═N—C(—A—B′)═CH—, —CH═C(—A—B′)—N═CH—, —CH═CH—C(—A—B′)═N—, —N═C(—A—B′)—N═CH—, or —CH═N—C(—A—B′)═N— bridge

wherein

A is as hereinbefore defined, and

B′ has the meanings given for B hereinbefore with the proviso that B′ contains an R₆O—CO, (R₇O—PO—OR₈), or (R₇O—PO—R₉) group, wherein R₉ is as hereinbefore defined and at least one of the groups R₆ to R₈ does not represent a hydrogen atom, by hydrolysis, treating with acids, thermolysis, or hydrogenolysis into a compound of general formula I, wherein at least one of the groups R₆ to R₈ denotes a hydrogen atom.

For example, functional derivatives of the carboxyl group such as the unsubstituted or substituted amides, esters, thioesters, trimethylsilylesters, orthoesters, iminoesters, amidines, or anhydrides, or the nitrile group may be converted by hydrolysis into a carboxyl group,

ester with tertiary alcohols, e.g., the tert-butylester, may be converted by treatment with an acid or thermolysis into a carboxyl group, and

esters with aralkanols, e.g., the benzylesters, may be converted by hydrogenolysis into a carboxyl group.

The hydrolysis is conveniently carried out either in the presence of an acid such as hydrochloric acid, sulfuric acid, phosphoric acid, acetic acid, trichloroacetic acid, trifluoroacetic acid, or mixtures thereof, or in the presence of a base such as lithium hydroxide, sodium hydroxide, or potassium hydroxide in a suitable solvent such as water, water/methanol, water/ethanol, water/isopropanol, methanol, ethanol, water/tetraihydrofaran, or water/dioxane at temperatures between −10° C. and 120° C., e.g., at temperatures between ambient temperature and the boiling temperature of the reaction mixture.

Under the reaction conditions mentioned above, any N-acylamino or N-acylimino groups present, such as an N-trifluoroacetylimino group, may be converted into the corresponding amino or imino groups. Moreover, any alcoholic hydroxy groups present may be converted, during the treatment with an organic acid such as trichloroacetic acid or trifluoroacetic acid, into a corresponding acyloxy group such as the trifluoroacetoxy group.

If B′ in a compound of formula IV contains a cyano or aminocarbonyl group, these groups may also be converted into the carboxyl group with a nitrite, e.g., sodium nitrite, in the presence of an acid such as sulfuric acid, which is conveniently used as the solvent at the same time, at temperatures between 0° C. and 50° C.

If B′ in a compound of formula IV denotes the tert-butyloxycarbonyl group, for example, the tert-butyl group may also be cleaved by treating with an acid such as trifluoroacetic acid, formic acid, p-toluenesulfonic acid, sulfuric acid, hydrochloric acid, phosphoric acid, or polyphosphoric acid optionally in an inert solvent such as methylene chloride, chloroform, benzene, toluene, diethylether, tetrahydrofuran, or dioxane preferably at temperatures between −10° C. and 120° C., e.g., at temperatures between 0° C. and 60° C., or optionally thermally in an inert solvent such as methylene chloride, chloroform, benzene, toluene, tetrahydrofuran, or dioxane and preferably in the presence of a catalytic amount of an acid such as p-toluenesulfonic acid, sulfuric acid, phosphoric acid, or polyphosphoric acid preferably at the boiling temperature of the solvent used, e.g., at temperatures between 40° C. and 120° C. Under the reaction conditions mentioned, any N-tert-butyloxycarbonylamino or N-tert-butyloxycarbonylimino groups present may be converted into the corresponding amino or imino groups.

If B′ in a compound of formula IV contains the benzyloxycarbonyl group, for example, the benzyl group may also be hydrogenolytically cleaved in the presence of a hydrogenation catalyst such as palladium/charcoal in a suitable solvent such as methanol, ethanol, ethanol/water, glacial acetic acid, ethyl acetate, dioxane, or dimethylformamide preferably at temperatures between 0° C. and 50° C., e.g., ambient temperature, and at a hydrogen pressure of 1 to 5 bar. During the hydrogenolysis other groups may be converted at the same time, e.g., a nitro group into an amino group, a benzyloxy group into a hydroxy group and a N-benzylamino, N-benzylimino, N-benzyloxycarbonylamino, or N-benzyloxycarbonylimino group into a corresponding amino or imino group.

c) In order to prepare a compound of general formula I wherein A denotes an —NR₄—C₄₋₇-cycloalkylene-NH—SO₂—CH₂CH₂ or —NR₄—C₄₋₇-cycloalkylene-N(C₁₋₄-alkyl)—SO₂—CH₂CH₂ group and B denotes an R₆O—CO—C₁₋₆-alkylene-NR₅ group, wherein R₄ to R₆ are as hereinbefore defined:

reacting a compound of general formula

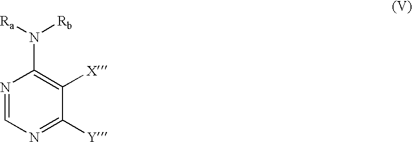

wherein

Rₐ and R_{b} are as hereinbefore defined,

X′″ and Y′″ together denote a —N═C(—A′—H)—CH═CH—, —CH═N—C(—A′—H)═CH—, —CH═C(—A′—H)—N═CH—, —CH═CH—C(—A′—H)═N—, —N═C(—A′—H)—N═CH—, or —CH═N—C(—A′—H)═N— bridge

wherein

A′ denotes an —NR₄—C₄₋₇-cycloalkylene-NH—SO₂—CH═CH₂, or —NR₄—C₄₋₇-cycloalkylene-N(C₁₋₄-alkyl)—SO₂—CH═CH₂ group, wherein R₄ is as hereinbefore defined, with a compound of general formula

R₆O—CO—C₁₋₆-alkylene-HNR₅ (VI)

wherein

R₅ and R₆ are as hereinbefore defined.

The reaction is preferably carried out in a solvent such as methanol, ethanol, or isopropanol in the presence of a base such as N-ethyl-diisopropylamine at temperatures between 0° C. and 100° C., but preferably at the boiling temperature of the reaction mixture.

d) In order to prepare a compound of general formula I wherein B denotes an R₆O—CO-alkylene-NR₅ group wherein the alkylene moiety, which is straight-chained and contains 1 to 6 carbon atoms, may additionally be substituted by one or two C₁₋₂-alkyl groups or by an R₆O—CO or R₆O—CO—C₁₋₂-alkyl group,

a piperazino or homopiperazino group substituted in the 4 position by an R₆O—CO—C₁₋₄-alkyl or bis(R₆O—CO)—C₁₋₄-alkyl group, or

a pyrrolidinyl, piperidinyl, or hexahydroazepinyl group substituted in the 1 position by an R₆O—CO—C₁₋₄-alkyl or bis(R₆O—CO)—C₁₋₄-alkyl group, wherein in each case R₅ and R₆ are as hereinbefore defined:

reacting a compound of general formula

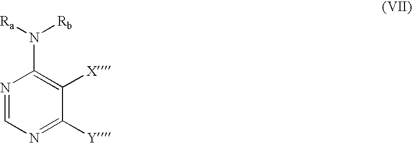

wherein:

Rₐ and R_{b} are as hereinbefore defined,

X″″ and Y″″ together denote a —N═C(—A—B″)—CH═CH—, —CH═N—C(—A—B″)═CH—, —CH═C(—A—B″)—N═CH—, —CH═CH—C(—A—B″)═N—, —N═C(—A—B″)—N═CH—, or —CH═N—C(—A—B″)═N— bridge,

wherein:

A is as hereinbefore defined, and

B″ denotes an R₅NH group wherein R₅ is as hereinbefore defined, a piperazino or homopiperazino group unsubstituted in the 4 position, a pyrrolidinyl, piperidinyl, or hexahydroazepinyl group unsubstituted in the 1 position, with a compound of general formula

R₆O—CO-alkylene-Z₂ (VIII)

wherein:

the alkylene moiety, which is straight-chained and contains 1 to 6 carbon atoms, may additionally be substituted by one or two C₁₋₂-alkyl groups or by an R₆O—CO or R₆—CO—C₁₋₂-alkyl group, wherein R₆ in each case is as hereinbefore defined, and Z₂ denotes an exchangeable group such as a halogen atom or a substituted sulfonyloxy group, e.g., a chlorine or bromine atom, a methylsulfonyloxy, propylsulfonyloxy, phenylsulfonyloxy, or benzylsulfonyloxy group.

The reaction is optionally carried out in a solvent or mixture of solvents such as methylene chloride, dimethylformamide, dimethylsulfoxide, sulfolane, benzene, toluene, chlorobenzene, tetrahydrofuran, benzene/tetrahydrofuran, or dioxane conveniently in the presence of a tertiary organic base such as triethylamine or N-ethyl-diisopropylamine (Hünig's base), wherein these organic bases may simultaneously serve as solvents, or in the presence of an inorganic base such as sodium carbonate, potassium carbonate, or sodium hydroxide solution conveniently at temperatures between −20° C. and 200° C., preferably at temperatures between 0° C. and 150° C.

e) In order to prepare a compound of general formula I wherein B denotes an (R₇O—PO—OR₈)—CH₂—NR₅ or (R₇O—PO-R₉)—CH₂—NR₅ group,

a piperazino or homopiperazino group substituted in the 4 position by an (R₇O—PO—OR₈)—CH₂ or (R₇O—PO—R₉)—CH₂ group, or

a pyrrolidinyl, piperidinyl, or hexahydroazepinyl group substituted in the 1 position by a (R₇O—PO—OR₈)—CH₂ or (R₇O—PO—R₉)—CH₂ group, wherein in each case R₅ and R₇ to R₉ are as hereinbefore defined:

reacting a compound of general formula

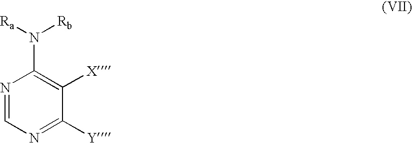

wherein

Rₐ and R_{b} are as hereinbefore defined,

X″″ and Y″″ together denote a —N═C(—A—B″)—CH═CH—, —CH═N—C(—A—B″)═CH—, —CH═C(—A—B″)—N═CH—, —CH═CH—C(—A—B″)═N—, —N═C(—A—B″)—N═CH—, or —CH═N—C(—A—B″)═N— bridge

wherein

A is as hereinbefore defined, and

B″ denotes an R₅NH group wherein R₅ is as hereinbefore defined, a piperazino or homopiperazino group unsubstituted in the 4 position, a pyrrolidinyl, piperidinyl, or hexahydroazepinyl group unsubstituted in the 1 position, with formaldehyde or one of the derivatives thereof and a compound of general formula

H—(R₇O)PO(OR₈) (IX) or

C₁₋₄-alkoxy-P(R₇O)(R₉) (X)

wherein R₇ to R₉ are as hereinbefore defined.

The reaction is conveniently carried out in a solvent or mixture of solvents such as dioxane, tetrahydrofuran, benzene, or toluene at temperatures between 50° C. and 150° C., preferably at the boiling temperature of the solvent used.

f) In order to prepare a compound of general formula I wherein B denotes an R₆O—CO—CH₂CH₂—NRs group wherein the —CH₂CH₂— moiety may be substituted by one or two C₁₋₂-alkyl groups or by an R₆O—CO or R₆—CO—C₁₋₂-alkyl group,

a piperazino or homopiperazino group substituted in the 4 position by an R₆O—CO—CH₂CH₂ group wherein the —CH₂CH₂- moiety may in each case additionally be substituted by an R₆O—CO or R₆O—CO—C₁₋₂-alkyl group, or

a pyrrolidinyl, piperidinyl, or hexahydroazepinyl group substituted in the I position by an R₆O—CO—CH₂CH₂ group wherein the —CH₂CH₂— moiety may in each case additionally be substituted by an R₆O—CO or R₆—CO—C₁₋₂-alkyl group and R₅ and R₆ in each case are as hereinbefore defined:

reacting a compound of general formula

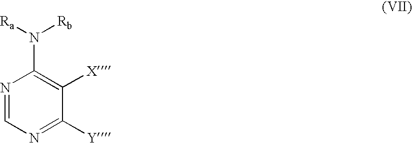

wherein

Rₐ and R_{b} are as hereinbefore defined,

X″″ and Y″″ together denote a —N═C(—A—B″)—CH═CH—, —CH═N—C(—A—B″)═CH—, —CH═C(—A—B″)—N═CH—, —CH═CH—C(—A—B″)═N—, —N═C(—A—B″)—N═CH—, or —CH═N—C(—A—B″)═N— bridge

wherein

A is as hereinbefore defined, and

B″ denotes an R₅NH group wherein R₅ is as hereinbefore defined, a piperazino or homopiperazino group unsubstituted in the 4 position, a pyrrolidinyl, piperidinyl, or hexahydroazepinyl group unsubstituted in the 1 position, with an acrylate of general formula

CH₂═CH—CO—OR₆ (XI)

wherein the vinyl moiety may be substituted by one or two C₁₋₂-alkyl groups or by an R₆O—CO or R₆—CO—C₁₋₂-alkyl group and R₆ in each case is as hereinbefore defined.

The reaction is preferably carried out in a solvent such as methanol, ethanol, or isopropanol at temperatures between 50° C. and 100° C., but preferably at the boiling temperature of the reaction mixture.

If according to the invention a compound of general formula I is obtained which contains a carboxy or hydroxyphosphoryl group, this may be converted by esterification into a corresponding ester of general formula I or

if a compound of general formula I is obtained wherein B denotes an optionally substituted N-(2-hydroxyethyl)-glycine or N-(2-hydroxyethyl)-glycine ester group, this group may be converted by cyclization into a corresponding 2-oxomorpholino compound.

The subsequent esterification is optionally carried out in a solvent or mixture of solvents such as methylene chloride, dimethylformamide, benzene, toluene, chlorobenzene, tetrahydrofuran, benzene/tetrahydrofuran, or dioxane, or particularly advantageously in a corresponding alcohol, optionally in the presence an acid such as hydrochloric acid or in the presence of a dehydrating agent, e.g., in the presence of isobutyl chloroformate, thionyl chloride, trimethylchlorosilane, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, or 1-hydroxybenzotriazole and optionally additionally in the presence of 4-dimethylamino-pyridine, N,N-carbonyldiimidazole, or triphenylphosphine/carbon tetrachloride, conveniently at temperatures between 0° C. and 150° C., preferably at temperatures between 0° C. and 80° C.

The subsequent ester formation may also be carried out by reacting a compound which contains a carboxy or hydroxyphosphoryl group with a corresponding alkyl halide.

The subsequent intramolecular cyclization is optionally carried out in a solvent or mixture of solvents such as acetonitrile, methylene chloride, tetrahydrofuran, dioxane, or toluene in the presence an acid such as hydrochloric acid or p-toluenesulfonic acid at temperatures between −10° C. and 120° C.

In the reactions described hereinbefore, any reactive groups present such as hydroxy, carboxy, phosphono, O-alkylphosphono, amino, alkylamino, or imino groups may be protected during the reaction by conventional protecting groups which are cleaved again after the reaction.

For example, a protecting group for a hydroxy group may be a trimethylsilyl, acetyl, benzoyl, methyl, ethyl, tert-butyl, trityl, benzyl, or tetrahydropyranyl group,

protecting groups for a carboxy group may be a trimethylsilyl, methyl, ethyl, tert-butyl, benzyl, or tetrahydropyranyl group,

protecting groups for a phosphono group may be an alkyl group such as the methyl, ethyl, isopropyl, or n-butyl group, the phenyl or benzyl group, and

protecting groups for an amino, alkylamino, or imino group may be a formyl, acetyl, trifluoroacetyl, ethoxycarbonyl, tert-butoxycarbonyl, benzyloxycarbonyl, benzyl, methoxybenzyl, or 2,4-dimethoxybenzyl group and additionally, for the amino group, a phthalyl group.

Any protecting group used is optionally subsequently cleaved for example by hydrolysis in an aqueous solvent, e.g., in water, isopropanol/water, acetic acid/water, tetrahydrofuran/water, or dioxane/water, in the presence of an acid such as trifluoroacetic acid, hydrochloric acid, or sulfuric acid or in the presence of an alkali metal base such as sodium hydroxide or potassium hydroxide or aprotically, e.g., in the presence of iodotrimethylsilane, at temperatures between 0° C. and 120° C., preferably at temperatures between 10° C. and 100° C. However, a benzyl, methoxybenzyl, or benzyloxycarbonyl group is cleaved, for example, hydrogenolytically, e.g., with hydrogen in the presence of a catalyst such as palladium/charcoal in a suitable solvent such as methanol, ethanol, ethyl acetate, or glacial acetic acid, optionally with the addition of an acid such as hydrochloric acid at temperatures between 0° C. and 100° C., but preferably at temperatures between 20° C. and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert-butyl or tert-butyloxycarbonyl group is preferably cleaved by treating with an acid such as trifluoroacetic acid or hydrochloric acid or by treating with iodotrimethylsilane optionally using a solvent such as methylene chloride, dioxane, methanol, or diethylether.

A trifluoroacetyl group is preferably cleaved by treating with an acid such as hydrochloric acid, optionally in the presence of a solvent such as acetic acid at temperatures between 50° C. and 120° C. or by treating with sodium hydroxide solution optionally in the presence of a solvent such as tetrahydrofuran at temperatures between 0° C. and 50° C.

A phthalyl group is preferably cleaved in the presence of hydrazine or a primary amine such as methylamine, ethylamine, or n-butylamine in a solvent such as methanol, ethanol, isopropanol, toluene/water, or dioxane at temperatures between 20° C. and 50° C.

A single alkyl group may be cleaved from an O,O′-dialkylphosphono group with sodium iodide, for example, in a solvent such as acetone, methyl ethyl ketone, acetonitrile, or dimethylformamide at temperatures between 40° C. and 150° C., but preferably at temperatures between 60° C. and 100° C.

Both alkyl groups may be cleaved from an O,O′-dialkylphosphono group with iodotrimethylsilane, bromotrimethylsilane, or chlorotrimethylsilane/sodium iodide, for example, in a solvent such as methylene chloride, chloroform, or acetonitrile at temperatures between 0° C. and the boiling temperature of the reaction mixture, but preferably at temperatures between 20° C. and 60° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf N. L. Allinger and E. L. Eliel in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.

The enantiomers are preferably separated by column separation on chiral phases or by recrystallization from an optically active solvent or by reacting with an optically active substance which forms salts or derivatives such as, e.g., esters or amides with the racemic compound, particularly acids and the activated derivatives or alcohols thereof, and separating the diastereomeric mixture of salts or derivatives thus obtained, e.g., on the basis of their differences in solubility, wherein the free antipodes may be released from the pure diastereomeric salts or derivatives by the action of suitable agents. Optically active acids in common use are, e.g., the D- and L-forms of tartaric acid or dibenzoyltartaric acid, di-o-tolyltartaric acid, malic acid, mandelic acid, camphorsulfonic acid, glutamic acid, aspartic acid, or quinic acid. An optically active alcohol may be, for example, (+) or (−)-menthol and an optically active acyl group in amides, for example, may be a (+)-or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulfuric acid, phosphoric acid, fumaric acid, succinic acid, lactic acid, citric acid, tartaric acid or maleic acid.

Moreover, if the new compounds of formula I thus obtained contain a carboxy, hydroxyphosphoryl, sulfo, or 5-tetrazolyl group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

The compounds of general formulae II to XI used as starting materials are known from the literature in some cases or may be obtained by methods known from the literature (cf. Examples I to XIX).

For example, a starting compound of general formulae II, IV, V, and VII is obtained by successively replacing exchangeable groups in a corresponding compound which is in turn obtained by known methods, e.g., by introducing halogen into a corresponding hydroxy compound.

A compound of general formula III is obtained by methods known from the literature, for example by reductive alkylation of a corresponding ketone, by alkylation of a corresponding amine, or by adding an amine to a corresponding alkenyl compound and optionally subsequently cleaving any protecting groups used.

As already mentioned hereinbefore, the compounds of general formula I according to the invention and their physiologically acceptable salts have valuable pharmacological properties, particularly an inhibiting effect on signal transduction mediated by the Epidermal Growth Factor receptor (EGF-R), wherein this may be achieved for example by inhibiting ligand bonding, receptor dimerization, or tyrosine kinase itself. It is also possible to block the transmission of signals to components located further down.

The biological properties of the new compounds were investigated as follows:

The inhibition of the EGF-R-mediated signal transmission can be demonstrated, e.g., with cells which express human EGF-R and whose survival and proliferation depend on stimulation by EGF or TGF-alpha. A cell line of murine origin dependent on interleukin-3-(IL-3) which was genetically modified to express functional human EGF-R was used here. The proliferation of these cells known as F/L-HERc can therefore be stimulated either by murine IL-3 or by EGF (cf T. von Ruiden et al., EMBO J. 7, 2749-2756 (1988) and J. H. Pierce et al., Science 239, 628-631 (1988)).

The starting material used for the F/L-HERc cells was the cell line FDC-P₁, the production of which has been described by T. M. Dexter et al., J. Exp. Med. 152. 1036-1047 (1980). Alternatively, however, other growth-factor-dependent cells may also be used (cf., e.g., J.H. Pierce et al., Science 239, 628-631 (1988); H. Shibuya et al., Cell 70, 57-67 (1992); and W. S. Alexander et al., EMBO J. 10, 3683-3691 (1991)). For expressing the human EGF-R cDNA (cf A. Ullrich et al., Nature 309, 418-425 (1984)) recombinant retroviruses were used as described by T. von Ruiden et al., EMBO J. 7, 2749-2756 (1988), except that the retroviral vector LXSN (cf A. D. Miller et al., BioTechniques 7, 980-990 (1989)) was used for the expression of the EGF-R cDNA and the line GP+E86 (cf D. Markowitz et al., J. Virol. 62, 1120-1124 (1988)) was used as the packaging cell.

The test was performed as follows:

F/L-HERc cells were cultivated in RPMI/1640 medium (BioWhittaker), supplemented with 10% fetal calf serum (FCS, Boehringer Mannheim), 2 mM glutamine (BioWhittaker), standard antibiotics and 20 ng/ml of human EGF (Promega), at 37° C. and 5% CO₂. In order to investigate the inhibitory activity of the compounds according to the invention, 1.5×10⁴ cells per well were cultivated in triplicate in 96-well plates in the above medium (200 μl), the cell proliferation being stimulated with either EGF (20 ng/ml) or murine IL-3. The IL-3 used was obtained from culture supernatants of the cell line X63/0 mIL-3 (cf H. Karasuyama et al., Eur. J. Immunol. 18, 97-104 (1988)). The compounds according to the invention were dissolved in 100% dimethylsulfoxide (DMSO) and added to the cultures in various dilutions, the maximum DMSO concentration being 1%. The cultures were incubated for 48 hours at 37° C.

In order to determine the inhibitory activity of the compounds according to the invention the relative cell number was measured in O.D. units using the Cell Titer 96™ Aqueous Non-Radioactive Cell Proliferation Assay (Promega). The relative cell number was calculated as a percentage of the control (F/LHERc cells without inhibitor) and the concentration of active substance which inhibits the proliferation of the cells by 50% (IC₅₀) was derived therefrom.

The following results were obtained:

| Compound | Inhibition of EGF-dependent proliferation |
|---|---|
| (Example No.) | IC₅₀ [nM] |
| 1 | 840 |
| 1(3) | 320 |
| 16 | 2300 |
| 1(8) | 1450 |
| 1(9) | 820 |
| 1(10) | 2510 |
| 1(11) | 2320 |
| 2(1) | 15 |
| 2(7) | 60 |
| 2(10) | 2040 |
| 2(12) | 810 |
| 2(13) | 1030 |
| 2(14) | 1150 |
| 2(15) | 1760 |
| 2(17) | 30 |
| 2(19) | 129 |
| 2(23) | 25 |
| 2(24) | 73 |
| 2(26) | 21 |
| 2(27) | 77 |
| 2(28) | 26 |
| 3(4) | 58 |
| 3(5) | 20 |
| 3(10) | 16 |
| 3(12) | 103 |
| 3(16) | 20 |
| 3(17) | 17 |
| 3(18) | 40 |
| 4(1) | 40 |
| 4(2) | 40 |
| 7 | 122 |

The compounds of general formula I according to the invention thus inhibit the signal transduction by tyrosine kinases, as demonstrated by the example of the human EGF receptor, and are therefore useful for treating pathophysiological processes caused by hyperfunction of tyrosine kinases. These are, e.g., benign or malignant tumors, particularly tumors of epithelial and neuroepithelial origin, metastasization and the abnormal proliferation of vascular endothelial cells (neoangiogenesis).

The compounds according to the invention are also useful for preventing and treating diseases of the airways and lungs which are accompanied by increased or altered production of mucus caused by stimulation by tyrosine kinases, e.g., in inflammatory diseases of the airways such as chronic bronchitis, chronic obstructive bronchitis, asthma, bronchiectasias, allergic, or non-allergic rhinitis or sinusitis, cystic fibrosis, α1-antitrypsin deficiency, or coughs, pulmonary emphysema, pulmonary fibrosis, and hyperreactive airways.

The compounds are also suitable for treating diseases of the gastrointestinal tract and bile duct and gall bladder which are associated with disrupted activity of the tyrosine kinases, such as may be found, e.g., in chronic inflammatory changes such as cholecystitis, Crohn's disease, ulcerative colitis, and ulcers in the gastrointestinal tract or such as may occur in diseases of the gastrointestinal tract which are associated with increased secretions, such as Menetrier's disease, secreting adenomas and protein loss syndrome, and also for treating nasal polyps and polyps of the gastrointestinal tract of various origins such as, e.g., villous or adenomatous polyps of the large bowel, but also polyps in familial polyposis coli, intestinal polyps in Gardner's syndrome, polyps throughout the entire gastrointestinal tract in Peutz-Jeghers syndrome, in inflammatory pseudopolyps, juvenile polyps, Colitis cystica profunda, and Pneumatosis cystoides intestinales.

Moreover, the compounds of general formula I and the physiologically acceptable salts thereof may be used to treat kidney diseases, particularly in cystic changes such as cystic kidneys, for treating renal cysts which may be idiopathic in origin or occur in syndromes such as, e.g., tuberculous sclerosis, in von-Hippel-Lindau Syndrome, in nephronophthisis and spongy kidney and other diseases caused by aberrant function of tyrosine kinases, such as, e.g., epidermal hyperproliferation (psoriasis), inflammatory processes, diseases of the immune system, hyperproliferation of hematopoietic cells, etc.

By reason of their biological properties the compounds according to the invention may be used on their own or in conjunction with other pharmacologically active compounds, for example in tumour therapy, in monotherapy or in conjunction with other anti-tumour therapeutic agents, for example in combination with topoisomerase inhibitors (e.g., etoposide), mitosis inhibitors (e.g., vinblastine), compounds which interact with nucleic acids (e.g., cisplatin, cyclophosphamide, adriamycin), hormone antagonists (e.g., tamoxifen), inhibitors of metabolic processes (e.g., 5-FU etc.), cytokines (e.g., interferons), antibodies, etc. For treating respiratory tract diseases, these compounds may be used on their own or in conjunction with other therapeutic agents for the airways, such as substances with a secretolytic, broncholytic and/or antiinflammatory activity. For treating diseases in the region of the gastrointestinal tract, these compounds may also be administered on their own or in conjunction with substances having an effect on motility or secretion or antiinflammatory substances. These combinations may be administered either simultaneously or sequentially.

These compounds may be administered either on their own or in conjunction with other active substances by intravenous, subcutaneous, intramuscular, intrarectal, intraperitoneal or intranasal route, by inhalation or transdermally or orally, wherein aerosol formulations are particularly suitable for inhalation.

For pharmaceutical use the compounds according to the invention are generally used for warm-blooded vertebrates, particularly humans, in doses of 0.01-100 mg/kg of body weight, preferably 0.1-15 mg/kg. For administration they are formulated with one or more conventional inert carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearyl alcohol, carboxymethylcellulose, or fatty substances such as hard fat or suitable mixtures thereof in conventional galenic preparations such as plain or coated tablets, capsules, powders, suspensions, solutions, sprays, or suppositories.

The following Examples are intended to illustrate the present invention without restricting it.

Preparation of the starting products:

### EXAMPLE I

4-amino-1-[(ethoxycarbonyl)methyl]piperidine dihydrochloride

Hydrogen chloride gas is passed through a solution of 2.36 g of 4-[(tert-butyloxycarbonyl)amino]-1-[(ethoxycarbonyl)methyl]piperidine in ethanol for about 10 minutes. The solution heats up significantly and after a short time a thick precipitate is formed. The suspension is refluxed for a further half hour, during which time the precipitate goes back into solution. The reaction mixture is concentrated by evaporation, taken up with toluene, and again concentrated by evaporation. The residue is stirred with acetone, suction filtered, and washed with acetone and diethylether. The almost colorless, crystalline product is dried in the desiccator. Yield: 2.15 g of (100% of theory); melting point: 156° C. (decomposition); mass spectrum (ESI⁺): m/z=187 [M+H]⁺.

The following compounds are obtained analogously to Example I:

(1) 4-amino-1-[(methoxycarbonyl)methyl]piperidine×4.4 trifluoroacetic acid

Carried out with trifluoroacetic acid in methylene chloride. ¹H-NMR (200 MHz, DMSO-d₆): *=1.7-2.0 (m, 2H), 2.0-2.2 (m, 2H), 3.0-3.4 (m, 3H), 3.45-3.65 (m, 2H), 3.75 (s, 3H), 4.2 (s, 2H), 8.25 (br s, 3H). Elemental analysis:

| | | | |
|---|---|---|---|
| calc: | C 29.94 | H 3.05 | N 4.16 |
| Found: | C 31.09 | H 3.65 | N 4.14 |

(2) 4-amino-1-[(propyloxycarbonyl)methyl]piperidine dihydrochloride

Melting point: 148° C.-154° C. (decomposition); mass spectrum (ESI⁺): m/z=201 [M+H]⁺.

(3) 4-amino-1-[(isopropyloxycarbonyl)methyl]piperidine dihydrochloride

Melting point: 159° C.-168° C.; mass spectrum (ESI⁺): m/z=201 [M+H]⁺.

(4) 4-amino-1-[(cyclohexyloxycarbonyl)methyl]piperidine×2 trifluoroacetic acid

Carried out with trifluoroacetic acid in methylene chloride. Melting point: 133° C.-138° C.; mass spectrum (ESI⁺): m/z=241 [M+H]⁺.

(5) 4-amino-1-[2-(methoxycarbonyl)ethyl]piperidine dihydrochloride

Melting point: 213° C.-215° C. (decomposition); mass spectrum (ESI⁺): m/z=187 [M+H]⁺.

(6) 4-amino-1-[3-(methoxycarbonyl)propyl]piperidine dihydrochloride

Melting point: 170° C.-172° C.; mass spectrum (EI): m/z=200 [M]⁺.

(7) trans-4-amino-1-{N-[(methoxycarbonyl)methyl]-N-methylamino }cyclohexane dihydrochloride

R_{f} value: 0.15 (silica gel, methylene chloride/methanol/concentrated, aqueous ammonia solution=90:10:1); mass spectrum (ESI⁺): m/z=201 [M+H]⁺.

(8) trans-4-amino-1-{N-[2-(methoxycarbonyl)ethyl]-N-methylamino}cyclohexane dihydrochloride

R_{f} value: 0.16 (silica gel, methylene chloride/methanol/concentrated, aqueous ammonia solution=90:10:1); mass spectrum (ESI⁺): m/z=215 [M+H]⁺.

(9) trans-4-amino-1-{N-[3-(methoxycarbonyl)propyl]-N-methylamino }cyclohexane dihydrochloride

Melting point: 170° C.-190° C. (decomposition); mass spectrum (ESI⁺): m/z 229 [M+H]⁺.

(10) 1-{1-[2-(ethoxycarbonyl)ethyl]piperidin-4-yl}piperazine×3 trifluoroacetic acid

Carried out with trifluoroacetic acid in methylene chloride. Melting point: 183° C.-186° C. (decomposition). Elemental analysis:

| | | | |
|---|---|---|---|
| calc: | C 39.29 | H 4.95 | N 6.87 |
| Found: | C 39.01 | H 4.97 | N 7.03 |

(11) 4-({N-[(methoxycarbonyl)methyl]-N-methylamino}methyl)piperidine×2 trifluoroacetic acid

Carried out with trifluoroacetic acid in methylene chloride.

(12) 4-{[2-(methoxycarbonyl)piperidine-1-yl]methyl}piperidine×2 trifluoroacetic acid

Carried out with trifluoroacetic acid in methylene chloride. R_{f} value: 0.30 (silica gel, methylene chloride/methanol/concentrated aqueous ammonia solution=90:10:0.1).

(13) 4-{[2-(methoxycarbonyl)-pyrrolidin-1-yl]methyl}piperidine×2 trifluoroacetic acid

Carried out with trifluoroacetic acid in methylene chloride. R_{f} value: 0.13 (silica gel, methylene chloride/methanol/concentrated aqueous ammonia solution=90:10:0.1).

(14) 4-({4-[(ethoxycarbonyl)methyl]piperazin-1-yl}methyl)piperidine×2 trifluoroacetic acid

Carried out with trifluoroacetic acid in methylene chloride. R_{f} value: 0.18 (silica gel, methylene chloride/methanol/concentrated aqueous ammonia solution=90:10:0.1).

### EXAMPLE II

1-[(ethoxycarbonyl)methyl]-4-(2-aminoethyl)piperidine dihydrochloride

1.0 g of 1-[(ethoxycarbonyl)methyl]-4-(cyanomethyl)piperidine hydrochloride is dissolved in 15 ml ethanol and 1.0 ml of ethanolic hydrochloric acid and hydrogenated in the presence of 0.15 g of palladium (10% on activated charcoal) as catalyst at 50° C. at a hydrogen pressure of 50 psi in a Parr apparatus until the calculated amount of hydrogen is taken up. The catalyst is filtered off and the filtrate is concentrated by evaporation. The residue is taken up in acetone and ethanolic hydrochloric acid is added dropwise until the dihydrochloride is precipitated. The precipitate is suction filtered, washed with acetone and diethylether, and dried in the desiccator. Yield: 760 mg (66% of theory); R_{f} value: 0.22 (silica gel, toluene/dioxane/methanol/concentrated, aqueous ammonia solution=20:50:20:2)

### EXAMPLE III

3-{4-[2-(methoxycarbonyl)ethyl]piperdin-1-yl} -pyrrolidine dihydrochloride

5.3 g of 4-[2-(methoxycarbonyl)ethyljpiperidine and 2.07 g of sodium acetate are added to a solution of 4.4 g of N-benzyl-3-pyrrolidinone in 45 ml methanol. Then 1.61 g of sodium cyanoborohydride is added and the reaction mixture is stirred for three days at ambient temperature. For working up, the reaction mixture is concentrated by evaporation and the residue is stirred with saturated sodium hydrogen carbonate solution. The aqueous phase is extracted with ethyl acetate, the combined extracts are washed with water and saturated sodium chloride solution, dried over sodium sulfate, and concentrated by evaporation. The crude product is purified by chromatography over a silica gel column with methylene chloride/methanol (9:1). Yield: 5.60 g (67% of theory) of N-benzyl-3-{4-[2-(methoxy-carbonyl)ethyl]piperdin-1-yl}-pyrrolidine as a yellowish oil; R_{f} value: 0.54 (silica gel, methylene chloride/methanol=9:1).

In order to cleave the benzyl protecting group 5.4 g of the product obtained are dissolved in 100 ml methanol, acidified with IN hydrochloric acid and hydrogenated in the presence of 1.5 g of palladium (10% on activated charcoal) at ambient temperature at a hydrogen pressure of 50 psi in a Parr apparatus. The catalyst is filtered off, the filtrate is concentrated by evaporation and the brownish crystalline product is dried in the desiccator. Yield: 5.10 g (100% of theory); R_{f} value: 0.56 (reversed phase ready-made thin layer plate RP-8 (E. Merck), methanol/5% aqueous sodium chloride solution =6:4).

### EXAMPLE IV

4-[(tert-butyloxycarbonyl)aminol-1-[(ethoxycarbonyl)methyl]piperidine

1.36 ml of ethyl bromoacetate and 2.77 ml of triethylamine are added to 2.00 g of 4-[(tert-butyloxycarbonyl)amino]piperidine in 15 ml acetonitrile at ambient temperature. The reaction mixture is stirred at 65° C. for about two hours, during which time a clear solution is formed. The solvent is distilled off using a rotary evaporator, the residue is stirred with ice-cold water and made alkaline with a little potassium carbonate solution. The precipitate thus formed is suction filtered and the aqueous phase is extracted with ethyl acetate. The combined extracts are washed with water and saturated sodium chloride solution, dried over magnesium sulfate, and concentrated by evaporation. The residue is combined with the precipitate filtered off, washed with water, and dried in the desiccator. Yield: 2.40 g (84% of theory); melting point: 76-79° C.; mass spectrum (ESI⁺): 309 [M+Na]⁺.

The following compounds are obtained analogously to Example IV:

(1) 4-[(tert-butyloxycarbonyl)amino]-1-[(methoxycarbonyl)methyl]piperidine

Melting point: 96° C.-98° C.; R_{f} value: 0.21 (silica gel, cyclohexane/ethyl acetate=1:1)

(2) 4-[(tert-butyloxycarbonyl)amino]-1-[(propyloxycarbonyl)methyl]piperidine

Melting point: 97° C.-99° C.; mass spectrum (ESI⁺): 323 [M+Na]⁺.

(3) 4-[(tert-butyloxycarbonyl)amino]-1-[(isopropyloxycarbonyl)methyl]piperidine

Melting point: 94° C.-96° C.; mass spectrum (ESI⁺): 323 [M+Na]⁺.

(4) 4-[(tert-butyloxycarbonyl)amino]-1-[(cyclohexyloxycarbonyl)methyl]piperidine

Melting point: 102° C.-104° C.; mass spectrum (ESI⁺): 363 [M+Na]⁺.

(5) 4-[(tert-butyloxycarbonyl)amino]-1-[3-(methoxycarbonyl)propyl]piperidine

R_{f} value: 0.75 (silica gel, methylene chloride/methanol/concentrated aqueous ammonia solution=90:10:0.1); mass spectrum (ESI⁺): 301 [M+H]⁺.

(6) trans-4-[(tert-butyloxycarbonyl)amino]1-{N-[(methoxycarbonyl)methyl]-N-methylamino}cyclohexane

R_{f} value: 0.65 (silica gel, methylene chloride/methanol/concentrated aqueous ammonia solution=90:10:1); mass spectrum (ESI⁺): 301 [M+H]⁺.

(7) trans-4-[(tert-butyloxycarbonyl)amino]-1-{N-[3-(methoxycarbonyl)propyl]-N-methylamino}cyclohexane

R_{f} value: 0.50 (silica gel, methylene chloride/methanol/concentrated aqueous ammonia solution=90:10:1); mass spectrum (ESI⁺): 329 [M+H]⁺.

(8) 1-[(ethoxycarbonyl)methyl]-4-(cyanomethyl)piperidine hydrochloride (after reacting the crude product obtained to form the hydrochloride)

Melting point: 131° C.-136° C.; R_{f} value: 0.67 (silica gel, methylene chloride/methanol/concentrated aqueous ammonia solution=95:5:1).

### EXAMPLE V

4-[(tert-butyloxycarbonyl)amino]-1-[2-(methoxycarbonyl)ethyl]piperidine

6.45 g of methyl acrylate is added to 5.00 g of 4-[(tert-butyloxycarbonyl)amino]piperidine in 20 ml methanol. The reaction mixture is stirred for 7.5 hours at 70° C. After the reaction has ended, the reaction mixture is concentrated by evaporation, leaving a white solid. Yield: 7.09 g (99% of theory); melting point: 91-93° C.; mass spectrum (ESI⁺): 287 [M+H]⁺.

The following compounds are obtained analogously to Example V:

(1) trans-4-[(tert-butyloxycarbonyl)amino]-1-{N-[2-(methoxycarbonyl)ethyl]-N-methylamino}cyclohexane

R_{f} value: 0.55 (silica gel, methylene chloride/methanol/concentrated aqueous ammonia solution=90:10:1); mass spectrum (ESI⁺): 315 [M+H]⁺.

(2) 1-{1-[2-(ethoxycarbonyl)ethyl]piperidin-4-yl}-4-(tert-butyloxycarbonyl)piperazine

R_{f} value: 0.29 (silica gel, methylene chloride/methanol/concentrated aqueous ammonia solution=95:5:1)

### EXAMPLE VI

trans-4-[(tert-butyloxyearbonyl)amino]-1-(methylamino)cyclohexane

A suspension of 26.30 g of trans-4-[(tert-butyloxycarbonyl)amino]-1-[N-(trifluoromethylcarbonyl)-N-methylamino]cyclohexane in 250 ml methanol is heated to 50° C. with stirring for a few minutes, until a clear solution is formed. Then 50 ml of 2N sodium hydroxide solution is added with stirring. A slightly cloudy solution is formed which is stirred for a further 2.5 hours at ambient temperature. The reaction mixture is concentrated by evaporation, the residue is taken up in 2N citric acid solution and extracted with methylene chloride/methanol (9:1). Then it is made alkaline with 2N sodium hydroxide solution and extracted again with methylene chloride/methanol (9:1). The combined extracts are dried over magnesium sulfate and concentrated by evaporation. Yield: 16.00 g (86% of theory); melting point: 120° C.-122° C.; mass spectrum (ESI⁺): 229 [M+H]⁺.

### EXAMPLE VII

trans-4-[(tert-butyloxycarbonyl)amino]1-[N-(trifluoromethylcarbonyl)-N-methylaminolcyclohexane

4.54 g of sodium hydride at ambient temperature is added in batches with stirring to a suspension of 27.10 g of trans-4-[(tert-butyloxycarbonyl)amino]-1-[(trifluoromethylcarbonyl)amino]cyclohexane in 220 ml of dimethylformamide. The slightly cloudy reaction solution is stirred for approximately a further 20 minutes at ambient temperature, then 6.47 ml of methyl iodide is added dropwise while cooling with an ice bath, whereupon a colorless precipitate slowly settles out. The reaction mixture is stirred overnight at ambient temperature and then poured onto 750 ml of ice-cold water for working up and neutralized with citric acid. The precipitate formed is filtered off, washed with water and dried in the desiccator. Yield: 26.40 g (93% of theory); melting point: 158° C.-166° C.; R_{f} value: 0.75 (silica gel, methylene chloride/methanol=95:5).

### EXAMPLE VIII

trans-4-[(tert-butyloxycarbonyl)amino]-1-[(trifluoromethylcarbonyl)amino]cyclohexane 10.56 ml of methyl trifluoroacetate are quickly added dropwise to 22.10 g of 1-amino-4-[(tert-butyloxycarbonyl)amino]cyclohexane in 110 ml methanol while cooling with an ice bath, whereupon a white precipitate is formed. Then the ice bath is removed and the reaction mixture is stirred for a further 3.5 hours at ambient temperature. The precipitate formed is filtered off, washed with 50 ml ice-cold methanol and a little diethylether, and dried in the desiccator. Yield: 27.26 g (85% of theory); melting point: 245° C.-246° C. (decomposition); R_{f} value: 0.4 (silica gel, methylene chloride/methanol=95:5).

### EXAMPLE IX

N-(3-aminopropyl)-sarcosine ethyl ester hydrochloride 20 ml trifluoroacetic acid is added dropwise to a solution of 6.10 g of N-[3-(tert-butyloxycarbonylamino)-propyl]-sarcosine ethylester in 40 ml methylene chloride while cooling with an ice bath. The reaction mixture is then stirred for about another three hours at 0° C. until the development of gas has ceased. For working up, the solvent is substantially distilled off in vacuo using the rotary evaporator. The residue is taken up in ethereal hydrochloric acid solution and again concentrated to dryness by evaporation. Yield: 4.72 g (86% of theory); R_{f} value: 0.80 (silica gel, acetonitrile/water/trifluoroacetic acid=50:50:1); mass spectrum (EI): m/z=174 [M]⁺.

### EXAMPLE X

N-[3-(tert-butyloxycarbonylamino)propyl]sarcosine ethyl ester

A solution of 17.90 g of 3-(tert-butyloxycarbonylamino)propyl bromide in 50 ml acetonitrile is added dropwise, within 30 minutes, to a mixture of 11.55 g of sarcosine ethyl ester hydrochloride and 28.8 ml of Huinig's base in 200 ml acetonitrile while cooling with an ice bath. The reaction mixture is allowed to come back up to ambient temperature overnight in the ice bath. Then the solvent is distilled off using a rotary evaporator, the residue is taken up in tert-butyl methyl ether, and washed with ice-cold water. The organic phase is dried over magnesium sulfate and concentrated by evaporation. The crude product is chromatographed on a silica gel column with methylene chloride/methanol/concentrated aqueous ammonia solution (100:2:0.1). Yield: 20.62 g (30% of theory); R_{f} value: 0.50 (silica gel, methylene chloride/methanol/concentrated aqueous ammonia solution=20:1:0.1); mass spectrum (ESI⁺): m/z=275 [M+H]⁺.

### EXAMPLE XI

4-[(3-chloro-4-fluorophenyl)amino]-6-{4-[(tert-butyloxycarbonyl)amino]-4-(methoxycarbonyl)piperidin-1-yl}pyrimido[5,4-d]pyrimidine

1.03 g of 4-[(tert-butyloxycarbonyl)amino]-4-(methoxycarbonyl)piperidine is added to 676 mg of 4-[(3-chloro-4-fluorophenyl)amino]-6-methylsulfinylpyrimido[5,4-d]pyrimidine and 0.42 ml triethylamine in 10 ml dioxane and the reaction mixture is refluxed for one hour. The reaction solution is concentrated by evaporation and the residue taken up in methylene chloride. The solution is washed with dilute potassium carbonate solution and water, dried over magnesium sulfate, and concentrated by evaporation. The crude product is purified by chromatography over a silica gel column with methylene chloride/methanol (98:2). Yield: 750 mg (71% of theory); melting point: 186° C.-189° C. (decomposition); mass spectrum (ESI⁺): m/z=532, 534 [M+H]⁺.

The following compounds are obtained analogously to Example XI:

(1) 4-[(3-chloro-4-fluorophenyl)amino]-6-(N— {trans-4-[(tert-butyloxycarbonyl)amino]cyclohex-1-yl}-N-methylamino)pyrimido[5,4-d]pyrimidine

Melting point: 202.5° C.-204.5° C.; mass spectrum (ESI⁺): m/z=502, 504 [M+H]⁺.

(2) 4-[(3-chloro-4-fluorophenyl)amino]-6-[N-(trans-4-methylaminocyclohex-1-yl)-N-methylamino]pyrimido[5,4-d]pyrimidine

R_{f} value: 0.30 (silica gel, toluene/dioxane/methanol/concentrated aqueous ammonia solution=20:50:20:10); mass spectrum (ESI⁺): m/z=416, 418 [M+H]⁺.

(3) 4-[(3-bromophenyl)amino]-6-{[1-(tert-butyloxycarbonyl)piperidin-4-yl]amino}pyrimido[5,4-d]pyrimidine

Melting point: 205° C.; mass spectrum (ESI⁺): m/z=500, 502 [M+H]⁺.

(4) 4-[(3-bromophenyl)amino]-6-{[1-(tert-butyloxycarbonyl)piperidine-3-yl]amino}pyrimido[5,4-d]pyrimidine

Melting point: 218° C. (decomposition); mass spectrum (EI): m/z=499, 501 [M]⁺.

### EXAMPLE XII

4-[(tert-butyloxycarbonyl)amino]-4-(methoxycarbonyl)piperidine 2.44 g of 1-benzyl-4-[(tert-butyloxycarbonyl)amino]-4-(methoxycarbonyl)piperidine in 20 ml methanol are hydrogenated in the presence of 300 mg palladium (10% on activated charcoal) as catalyst at ambient temperature at a hydrogen pressure of 50 psi for about 22 hours until the calculated amount of hydrogen is taken up. The catalyst is filtered off and the filtrate concentrated by evaporation. Yield: 1.72 g (95% of theory); R_{f} value: 0.15 (silica gel, methylene chloride/methanol/concentrated aqueous ammonia solution=90:10:1); mass spectrum (ESI⁺): m/z=259 [M+H]⁺.

### EXAMPLE XIII

1-benzyl-4-[(tert-butyloxycarbonyl)amino]-4-(methoxycarbonyl)piperidine

3.97 g of di-tert-butyl pyrocarbonate is added to a suspension of 5.05 g of 4-amino-1-benzyl-4-(methoxycarbonyl)piperidine in 80 ml methylene chloride. Then 16 ml of 2N sodium hydroxide solution is added dropwise, with stirring, at ambient temperature, whereupon a precipitate is formed which is in the aqueous phase. After one hour the organic phase is separated off, dried over magnesium sulfate, and concentrated by evaporation. Since the crude product mixture obtained still contains starting material, it is dissolved in 30 ml tetrahydrofuran, mixed with 1.50 g of di-tert-butyl pyrocarbonate and a spatula tip of 4-dimethylaminopyridine, and refluxed for three hours. The reaction mixture is concentrated by evaporation, leaving a brown resin which is reacted without any further purification. Yield: 2.64 g (48% of theory); R_{f} value: 0.65 (silica gel, methylene chloride/methanollconcentrated aqueous ammonia solution=90:10:1); mass spectrum (EI): m/z=348 [M]⁺.

### EXAMPLE XIV

1-(tert-butyloxycarbonyl)-4-({N-[(methoxycarbonyl)methyl]-N-methylamino}methyl)piperidine

First, 11.0 g of sarcosine methyl ester hydrochloride are converted into the free base by treating with 10-15% potassium carbonate solution. This is then heated to 110° C. together with 2.0 g of (1-tert-butyloxycarbonyl)-4-[(methylsulfonyloxy)methyl]piperidine in a pressurised vessel for six hours at a pressure of 2 bar. Then the reaction mixture is rinsed out of the pressurised vessel with methanol and concentrated by evaporation. A brown oil is left which is stirred with a little water. The aqueous phase is separated off and the organic phase is diluted with methylene chloride, dried over sodium sulfate, and freed from solvent using a rotary evaporator. The crude product obtained is reacted without any further purification. Yield: 2.49 g of brownish oil.

The following compounds are obtained analogously to Example XIV:

(1) 1-tert-butyloxycarbonyl-4-{[2-(methoxycarbonyl)piperidin-1-yl]methyl}piperidine

R_{f} value: 0.86 (silica gel, petroleum ether/ethyl acetate/methanol=10:10:1) Mass spectrum (ESI⁺): m/z=341 [M+H]⁺.

(2) 1-tert-butyloxycarbonyl-4-{[2-(methoxycarbonyl)pyrrolidin-1-yl]methyl}piperidine

R_{f} value: 0.74 (silica gel, petroleum ether/ethyl acetate/methanol=10:10:1); mass spectrum (ESI⁺): m/z=327 [M+H]⁺.

(3) 1-tert-butyloxycarbonyl-4-({4-[(ethoxycarbonyl)methyl]piperazin-1-yl}methyl)piperidine

R_{f} value: 0.69 (silica gel, methylene chloride/methanol=9:1); mass spectrum (ESI⁺): m/z=370 [M+H]⁺.

### EXAMPLE XV

4-[(3-chloro-4-fluorophenyl)amino]-6-({trans-4-[(2-hydroxyethyl)amino]cyclohex-1-yl}amino)pyrimido [5,4-d]pyrimidine

0.23 ml of 2-bromoethanol and 0.61 ml of diisopropylethylamine is added to 1.16 g of 4-[(3-chloro-4-fluorophenyl)amino]-6-[(trans-4-aminocyclohex-1-yl)amino]pyrimido[5,4-d]pyrimidine in 8 ml acetonitrile at ambient temperature. The resulting mixture is refluxed. After about 5 hours, another 0.05 ml of 2-bromoethanol is added and the mixture is heated for another eight hours to complete the reaction. The suspension is concentrated by evaporation, the residue is mixed with ice-cold water, made slightly alkaline with sodium hydroxide solution, and suction filtered. The still moist filter residue is taken up in methylene chloride/methanol. The cloudy solution is washed with water, dried over magnesium sulfate, and concentrated by evaporation. The yellow crude product is stirred with about 30 ml methanol, briefly heated to boiling, cooled slightly, suction filtered, and washed with cold methanol. Yield: 990 mg (76% of theory); melting point: 165° C.-172° C.; mass spectrum (ESI⁺): m/z=432, 434 [M+H]⁺.

The following compound is obtained analogously to Example XV:

(1) 4-[(3-chloro-4-fluorophenyl)amino]-6-(4-{[(2-hydroxyethyl)amino]methyl}piperidin-1-yl)pyrimido[5,4-d]pyrimidine

R_{f} value: 0.50 (silica gel, toluene/dioxane/methanol/concentrated aqueous ammonia solution=20:50:20:3); mass spectrum (ESI⁺): m/z=432, 434 [M+H]⁺.

### EXAMPLE XVI

4-[(3-chloro-4-fluorophenyl)amino]-6-[N-(trans-4-aminocyclohex1-yl)-N-methylamino]pyrimido[5,4-d]pyrimidine

3.0 ml trifluoroacetic acid is added dropwise to 2.10 g of 4-[(3-chloro-4-fluorophenyl)amino]-6-(N-{trans-4-[(tert-butyloxycarbonyl)amino]cyclohex-1-yl}-N-methylamino)pyrimido[5,4-d]pyrimidine in 30 ml methylene chloride. The reaction mixture is stirred for 1.5 hours at ambient temperature, left to stand overnight, and concentrated by evaporation the next morning. The residue is taken up in methylene chloride/methanol (5:1), washed with 2N sodium hydroxide solution and water, dried over magnesium sulfate, and concentrated by evaporation. The yellow crude product is triturated with diethyl ether, suction filtered, and dried in vacuo. Yield: 1.60 g (95% of theory); melting point: 203° C.-205° C.; mass spectrum (ESI⁺): m/z=402, 404 [M+H]⁺.

The following compounds are obtained analogously to Example XVI:

(1) 4-[(3-bromophenyl)amino]-6-[(piperidin-4-yl)amino]pyrimido[5,4-d]pyrimidine

Melting point: 215° C.; mass spectrum (ESI⁺): m/z=400, 402 [M+H]⁺.

(2) 4-[(3-bromophenyl)amino]-6-[(piperidin-3-yl)amino]pyrimido[5,4-d]pyrimidine

Melting point: 178° C.; mass spectrum (ESI⁺): m/z=400, 402 [M+H]⁺.

### EXAMPLE XVII

4-[(3-chloro-4-fluorophenyl)amino]-6-({trans-4-[(vinylsulfonyl)amino]cyclohex-1-yl}lamino)p=rimido[5.4-d]pyrimidine

0.38 ml of diisopropylethylamine is added to 388 mg of 4-[(3-chloro-4-fluorophenyl)amino]-6-[(trans-4-aminocyclohex-1-yl)amino]pyrimido[5,4-d]pyrimidine in 25 ml of tetrahydrofuran. The mixture is cooled to −55° C. under a nitrogen atmosphere in a bath of acetone and dry ice. Then a solution of 0.13 ml chloroethane sulfonic acid chloride in 5 ml of tetrahydrofuran is added dropwise and stirred for a further 1.5 hours at −55° C. The reaction mixture is quenched with a mixture of 10 ml of IN hydrochloric acid and 10 ml of saturated sodium chloride solution and mixed with some ethyl acetate. The organic phase is filtered through 8.5 g of EXTRELUT® (E. Merck, Darmstadt) and eluted with 100 ml of methylene chloride/methanol (9:1). The filtrate is concentrated by evaporation, leaving a yellow solid. Yield: 216 mg (45% of theory); melting point: 226-230° C. (decomposition); mass spectrum (EI): m/z=477, 479 [M]⁺.

### EXAMPLE XVIII

(R)-4-[(1-phenylethyl)amino]-6-methylsulfinylpyrimido[5.4-d]pyrimidine and (R)-4-[(1-phenylethyl)amino]-6-methylsulfonylpyrimido[5.4-d]pyrimidine

28.80 g of 3-chloroperbenzoic acid (content: 70%) is added batchwise, with stirring, to 17.40 g of (R)-4-[(1-phenylethyl)amino]-6-methylthiopyrimido[5,4-d]pyrimidine in 180 ml methylene chloride at ambient temperature. Then the reaction mixture is stirred for about an hour at ambient temperature. The white precipitate formed is filtered off and the filtrate is washed with sodium hydrogen carbonate solution, dried over magnesium sulfate, and concentrated by evaporation. The oily orange residue is a mixture of sulfone and sulfoxide (about 85:15 according to ¹H-NMR). R_{f} value: 0.47 (silica gel, cyclohexane/ethyl acetate/methanol 5:4:1); mass spectrum (ESI⁺): m/z=352 [M+Na]⁺(sulfone), 336 [M+Na]+(sulfoxide).

### EXAMPLE XIX

(R)-4-[(1-phenylethyl)amino]-6-methylthiopyrimido [5.4-d]pyrimidine

10.7 ml of diisopropylethylamine and 9.4 ml of D(+)-1-phenylethylamine are added to 13.00 g of 4-chloro-6-methylthiopyrimido[5,4-d]pyrimidine in 100 ml of dimethylformamide. The mixture is stirred for four hours at ambient temperature. For working up, the reaction mixture is poured onto 200 ml of water. The aqueous phase is extracted with methylene chloride, and the combined organic phases are dried over magnesium sulfate and concentrated by evaporation. The dark brown oily residue is taken up in ethyl acetate and extracted with 10% citric acid. The organic phase is dried over magnesium sulfate and concentrated by evaporation, leaving a reddish-brown oil. Yield: 17.40 g (96% of theory); R_{f} value: 0.63 (silica gel, cyclohexane/ethyl acetate/methanol=5:4:1); mass spectrum (ESI⁺): m/z=298 [M+H]⁺.

Preparation of the end products:

### EXAMPLE 1

4-[(3-chloro-4-fluorophenyl)amino]-6-{ [1-(carboxymethyl)piperidin-4-yl]amino}pyrimido[5.4-d]pyrimidine

2.0 ml of IN sodium hydroxide solution is added to a suspension of 400 mg of 4-[(3-chloro-4-fluorophenyl)amino]-6-({1-[(methoxycarbonyl)methyl]piperidin-4-yl}amino)pyrimido[5,4-d]pyrimidine in 5.0 ml tetrahydrofuran. The clear solution formed is stirred for approximately a further three hours at ambient temperature. Then the reaction solution is neutralized with 1N hydrochloric acid and concentrated by evaporation using a rotary evaporator until the product starts to crystallize out. The yellow precipitate is filtered off, washed with water and diethylether, and dried in vacuo at 60° C. Yield: 365 mg (96% of theory); melting point: 155° C. (decomposition); mass spectrum (EI): m/z=431, 433 [M]⁺.

The following compounds are obtained analogously to Example 1:

(1) 4-[(3-chloro-4-fluorophenyl)amino]-6-{[1-(2-carboxyethyl)piperidin-4-yl]amino}pyrimido[5,4-d]pyrimidine

Melting point: 217° C.-225° C.; mass spectrum (EI): m/z=445, 447 [M]⁺.

(2) 4-[(3-chloro-4-fluorophenyl)amino]-6-{[1-(3-carboxypropyl)piperidin-4-yl]amino}pyrimido[5,4-d]pyrimidine

Melting point: 145° C.-165° C.; mass spectrum (EI): m/z=459, 461 [M]⁺.

(3) 4-[(3-chloro-4-fluorophenyl)amino]-6-({trans-4-[N-(carboxymethyl)-N-methylamino]cyclohex-1-yl}amino)pyrimido[5,4-d]pyrimidine

Melting point: 220° C.-228° C.; mass spectrum (ESI⁺): m/z=460, 462 [M+H]⁺. (4) 4-[(3-chloro-4-fluorophenyl)amino]-6-({trans-4-[N-(2-carboxyethyl)-N-methylamino]cyclohex-1-yl}amino)pyrimido[5,4-d]pyrimidine

Melting point: 202° C.-205° C.; mass spectrum (ESI⁺): m/z=474, 476 [M+H]⁺.

(5) 4-[(3-chloro-4-fluorophenyl)amino]-6-({trans-4-[N-(3-carboxypropyl)-N-methylamino]cyclohex-1-yl}amino)pyrimido[5,4-d]pyrimidine

Melting point: 217° C.-221° C.; mass spectrum (ESI⁺): m/z=488, 490 [M+H]⁺.

(6) 4-[(3-chloro-4-fluorophenyl)amino]-6-[4-(carboxymethyl)piperazin-1-yl]pyrimido[5,4-d]pyrimidine

Melting point: 240° C. (decomposition); mass spectrum (EI): m/z=417, 419 [M]⁺.

(7) 4-[(3-chloro-4-fluorophenyl)amino]-6-[4-(2-carboxyethyl)piperazin-1-yl]pyrimido[5,4-d]pyrimidine

Melting point: 111° C.-145° C.; mass spectrum (EI): m/z=431, 433 [M]⁺.

(8) 4-[(3-chloro-4-fluorophenyl)amino]-6-{4-[1-(2-carboxyethyl)piperidin-4-yl]piperazin-1-yl}pyrimido[5,4-d]pyrimidine

Melting point: 213° C. (decomposition); mass spectrum (EI): m/z=514, 516 [M]⁺.

(9) 4-[(3-chloro-4-fluorophenyl)amino]-6-{2-[1-(carboxymethyl)piperidin-4-yl]ethylamino}pyrimido[5,4-d]pyrimidine

Melting point: 246° C.-249° C. (decomposition); mass spectrum (EI): m/z=459, 461 [M]⁺.

(10) 4-[(3-chloro-4-fluorophenyl)amino]-6-[2-(4-carboxypiperidin-1-yl)ethylamino]pyrimido [5,4-d]pyrimidine

Melting point: 190° C. (decomposition); mass spectrum (EI): m/z=445, 447 [M]⁺.

(11) 4-[(3-chloro-4-fluorophenyl)amino]-6-[4-[N-(2-carboxyethyl)-N-methylamino]piperidin-1-yl]pyrimido[5,4-d]pyrimidine

Melting point: 139° C.-165° C. (decomposition); mass spectrum (EI): m/z=459, 461 [M]+.

(12) 4-[(3-chloro-4-fluorophenyl)amino]-6-{3-[4-(2-carboxyethyl)piperdin-1-yl]-pyrrolidin-1-yl}pyrimido[5,4-d]pyrimidine

R_{f} value: 0.63 (silica gel, methylene chloride/methanol/triethylamine 2:1:0.1); mass spectrum , (EI): m/z=499, 501 [M]⁺.

(13) 4-[(3-chloro-4-fluorophenyl)amino]-6-{2-[4-(carboxymethyl)piperazin-1-yl]ethylamino}pyrimido[5,4-d]pyrimidine

Melting point: 240° C.-242° C. (decomposition); mass spectrum: (ESF⁻): m/z=459, 461 [M-H]⁻.

(14) 4-[(3-chloro-4-fluorophenyl)amino]-6-(4-amino-4-carboxypiperidin-1-yl)pyrimido[5,4-d]pyrimidine

Melting point: 277° C.-282° C.; mass spectrum (EI): m/z=417, 419 [M]⁺.

(15) 4-[(3-chloro-4-fluorophenyl)amino]-6-[4-(N-carboxymethyl-N-methylamino)piperidin-1-yl]pyrimido[5,4-d]pyrimidine

R_{f} value: 0.05 (silica gel, methylene chloride/methanol/concentrated aqueous ammonia solution=90:10:0.1); mass spectrum (ESI⁻): m/z=444, 446 [M-H]⁻.

(16) 4-[(3-chloro-4-fluorophenyl)amino]-6-(4-{[(2-carboxyethyl)amino]methyl}piperidin1-yl)pyrimido[5,4-d]pyrimidine

Melting point: 209° C.-214° C.; mass spectrum (ESI⁻): m/z=458, 460 [M-H]⁻.

(17) 4-[(3-chloro-4-fluorophenyl)amino]-6-(4-{[(carboxymethyl)amino]methyl}piperidin-1-yl)pyrimido[5,4-d]pyrimidine

Melting point: 226° C.-235° C.; mass spectrum (ESI⁻): m/z=444, 446 [M-H]⁻.

(18) 4-[(3-chloro-4-fluorophenyl)amino]-6-({trans-4-[N,N-bis(carboxymethyl)amino]cyclohex-1-yl}amino)pyrimido[5,4-d]pyrimidine

Melting point: 245° C. (decomposition); mass spectrum (ESI⁻): m/z=502, 504 [M-H]⁻.

(19) 4-[(3-chloro-4-fluorophenyl)amino]-6-(4-{[N,N-bis(2-carboxyethyl)amino]methyl}piperidin-1-yl)pyrimido[5,4-d]pyrimidine

Melting point: 160° C.-169° C.; mass spectrum (ESI⁻): m/z=530, 532 [M-H]⁻.

(20) 4-[(3-chloro-4-fluorophenyl)amino]-6-(3-{[N,N-bis(2-carboxyethyl)amino]methyl}piperidin-1-yl)pyrimido[5,4-d]pyrimidine

R_{f} value: 0.79 (Reversed phase ready-made TLC plate (E. Merck), acetonitrile/water/trifluoroacetic acid 90:10:1); mass spectrum (ESI⁻): m/z=530, 532 [M-H]⁻.

(21) 4-[(3-chloro-4-fluorophenyl)amino]-6-(4-{[N,N-bis(carboxymethyl)amino]methyl}piperidin-1-yl)pyrimido[5,4-d]pyrimidine

R_{f} value: 0.85 (Reversed phase ready-made TLC plate (E. Merck), acetonitrile/water/trifluoroacetic acid=90:10:1); mass spectrum (ESI⁻): m/z=502, 504 [M-H]⁻.

(22) 4-[(3-chloro-4-fluorophenyl)amino]-6-(N-{trans-4-[N′,N′-bis(carboxymethyl)amino]cyclohex-1-yl}-N-methylamino)pyrimido[5,4-d]pyrimidine

R_{f} value: 0.33 (Reversed phase ready-made TLC plate (E. Merck), methanol/5% aqueous sodium chloride solution=8:2); mass spectrum (ESI⁻): m/z=516, 518 [M-H]⁻.

(23) 4-[(3-chloro-4-fluorophenyl)amino]-6-(N-{trans-4-[(carboxymethyl)amino]cyclohex-1-yl}-N-methylamino)pyrimido[5,4-d]pyrimidine

R_{f} value: 0.30 (Reversed phase ready-made TLC plate (E. Merck), methanol/5% aqueous sodium chloride solution=8:2); mass spectrum (ESI⁻): m/z=558, 560 [M-H]⁻.

(24) 4-[(3-chloro-4-fluorophenyl)amino]-6-(3-{[(2-carboxyethyl)amino]methyl}piperidin-1-yl)pyrimido[5,4-d]pyrimidine

Melting point: 173° C.-179° C.; mass spectrum (ESI⁻): m/z=558, 560 [M-H]⁻.

(25) 4-[(3-chloro-4-fluorophenyl)amino]-6-(3-{[N,N-bis(carboxymethyl)amino]methyl}piperidin-1-yl)pyrimido[5,4-d]pyrimidine

R_{f} value: 0.82 (Reversed phase ready-made TLC plate (E. Merck), acetonitrile/water/trifluoroacetic acid=90:10:1); mass spectrum (ESI⁻): m/z=502, 504 [M-H]⁻.

(26) 4-[(3-chloro-4-fluorophenyl)amino]-6-(3-{[(carboxymethyl)amino]methyl}piperidin-1-yl)pyrimido[5,4-d]pyrimidine

R_{f} value: 0.82 (Reversed phase ready-made TLC plate (E. Merck), acetonitrile/water/trifluoroacetic acid=90:10:1); mass spectrum (ESI⁻): m/z=444, 446 [M-H]⁻.

(27) 4-[(3-chloro-4-fluorophenyl)amino]-6-({trans-4-[(carboxymethyl)amino]cyclohex-1-yl}amino)pyrimido [5,4-d]pyrimidine

Melting point: 201° C.-205° C. (decomposition); mass spectrum (ESI⁻): m/z=444, 446 [M-H]⁻.

(28) 4-[(3-chloro-4-fluorophenyl)amino]-6-(N-{trans-4-[N′-(carboxymethyl)-N′-methylamino]cyclohex-1-yl}-N-methylamino)pyrimido[5,4-d]pyrimidine

Melting point: 200° C. (decomposition); mass spectrum (ESI⁻): m/z=472, 474 [M-H]⁻.

(29) 4-[(3-chloro-4-fluorophenyl)amino]-6-{4-[(2-carboxypiperidin-1-yl)methyl]piperidine-1-y}pyrimido[5,4-d]pyrimidine

Carried out with potassium tert-butoxide as base. Melting point: 225-237° C. (decomposition); mass spectrum (ESI⁻): m/z=498, 500 [M-H]⁻.

(30) 4-[(3-chloro-4-fluorophenyl)amino]-6-{4-methyl-4-[(2-carboxyethyl)amino]piperidin-1-yl}pyrimido[5,4-d]pyrimidine

Melting point: 157-160° C.; mass spectrum (ESI⁻): m/z=458, 460 [M-H]⁻.

(31) 4-[(3-chloro-4-fluorophenyl)amino]-6-(4-{[4-(carboxymethyl)piperazin-1-yl]methyl}piperidin-1-yl)pyrimido[5,4-d]pyrimidine

R_{f} value: 0.60 (Reversed phase ready-made TLC plate (E. Merck), acetonitrile/water/trifluoroacetic acid=90:10:1); mass spectrum (ESI⁻): m/z=513, 515 [M-H]⁻.

(32) 4-[(3-chloro-4-fluorophenyl)amino]-6-{4-methyl-4-[(carboxymethyl)amino]piperidin-1-yl}pyrimido[5,4-d]pyrimidine

Melting point: 160° C. (decomposition); mass spectrum (ESI⁻): m/z=444, 446 [M-H]⁻. (33) 4-[(3-chloro-4-fluorophenyl)amino]-6-{4-[(2-carboxy-pyrrolidin-1-yl)methyl]piperidin-1-yl}pyrimido[5,4-d]pyrimidine

Carried out with potassium tert-butoxide as base. Melting point: 140-162° C. (decomposition); mass spectrum (ESI⁻): m/z=484, 486 [M-H]⁻.

### EXAMPLE 2

4-[(3-chloro-4-fluorophenyl)amino]-6-({1-[(ethoxycarbonyl)methyl]piperidin-4-yl}amino)pyrimido[5,4-d]pyrimidine 778 mg of 4-amino-1-[(ethoxycarbonyl)methyl]piperidine dihydrochloride is added to 676 mg of a mixture of 4-[(3-chloro-4-fluorophenyl)amino]-6-methylsulfinylpyrimido[5,4-d]pyrimidine and 4-[(3-chloro-4-fluorophenyl)amino]-6-methylsulfonylpyrimido[5,4-d]pyrimidine in 14 ml dioxane and 2 ml ethanol. Then 0.55 ml of triethylamine and 829 mg of potassium carbonate are added and the reaction mixture is refluxed for about seven hours. Then the reaction mixture is concentrated by evaporation and the residue is stirred with ice-cold water, suction filtered, washed with water, and dried. The brownish-yellow crude product is purified by chromatography on a silica gel column with methylene chloride/ethanol (95:5). Yield: 526 mg (57% of theory); melting point: 136-138° C.; Mass spectrum (EI): m/z=459, 461 [M]⁺.

The following compounds are obtained analogously to Example 2:

(1) 4-[(3-chloro-4-fluorophenyl)amino]-6-({1-[(methoxycarbonyl)methyl]piperidin-4-yl}amino)pyrimido [5,4-d]pyrimidine

Melting point: 162° C.-164° C.; mass spectrum (EI): m/z=445, 447 [M]⁺.

(2) 4-[(3-chloro-4-fluorophenyl)amino]-6-({1-[(propyloxycarbonyl)methyl]piperidin-4-yl}amino)pyrimido[5,4-d]pyrimidine

Melting point: 135° C.-137° C.; mass spectrum (EI): m/z=473, 475 [M]⁺.

(3) 4-[(3-chloro-4-fluorophenyl)amino]-6-({1-[(isopropyloxycarbonyl)methyl]piperidin-4-yl}amino)pyrimido[5,4-d]pyrimidine

Melting point: 175° C.-177° C.; mass spectrum (EI): m/z=473, 475 [M]⁺.

(4) 4-[(3-chloro-4-fluorophenyl)amino]-6-({1-[(cyclohexyloxycarbonyl)methyl]piperidin-4-yl}amino)pyrimido[5,4-d]pyrimidine

Melting point: 184° C.-186° C.; mass spectrum (EI): m/z=513, 515 [M]⁺.

(5) 4-[(3-chloro-4-fluorophenyl)amino]-6-({1-[2-(methoxycarbonyl)ethyl]piperidin-4-yl}amino)pyrimido[5,4-d]pyrimidine

Melting point: 136° C.-137° C.; mass spectrum (ESI⁺): m/z=460, 462 [M+H]⁺.

(6) 4-[(3-chloro-4-fluorophenyl)amino]-6-({1-[3-(methoxycarbonyl)propyl]piperidin-4-yl}amino)pyrimido[5,4-d]pyrimidine

Melting point: 135° C.-137° C.; mass spectrum (EI): m/z=473, 475 [M]⁺.

(7) 4-[(3-chloro-4-fluorophenyl)amino]-6-[(trans-4-{N-[(methoxycarbonyl)methyl]-N-methylamino}cyclohex-1-yl)amino]pyrimido[5,4-d]pyrimidine

Melting point: 131° C.-134° C.; mass spectrum (EI): m/z=473, 475 [M]⁺.

(8) 4-[(3-chloro-4-fluorophenyl)amino]-6-[(trans-4-{N-[2-(methoxycarbonyl)ethyl]-N-methylamino}cyclohex-1-yl)amino]pyrimido[5,4-d]pyrimidine

Melting point: 126° C.-128° C.; mass spectrum (EI): m/z=487, 489 [M]⁺.

(9) 4-[(3-chloro-4-fluorophenyl)amino]-6-[(trans-4-{N-[3-(methoxycarbonyl)propyl]-N-methylamino}cyclohex-1-yl)amino]pyrimido[5,4-d]pyrimidine

Melting point: 99° C.-102° C.; mass spectrum (ESI⁺): m/z=502, 504 [M+H]⁺.

(10) 4-[(3-chloro-4-fluorophenyl)amino]-6-[4-[(ethoxycarbonyl)methyl]piperazin-1-yl]pyrimido[5,4-d]pyrimidine

Melting point: 179° C.-182° C.; mass spectrum (EI): m/z=445, 447 [M]⁺.

(11) 4-[(3-chloro-4-fluorophenyl)amino]-6-[4-[2-(methoxycarbonyl)ethyl]piperazin-1-yl]pyrimido[5,4-d]pyrimidine

Melting point: 140° C.-142° C.; mass spectrum (EI): m/z=445, 447 [M]⁺.

(12) 4-[(3-chloro-4-fluorophenyl)amino]-6-(4-{1-[2-(ethoxycarbonyl)ethyl]piperidin-4-yl}piperazin-1-yl)pyrimido[5,4-d]pyrimidine

R_{f} value: 0.51 (silica gel, methylene chloride/methanol=9:1); mass spectrum (EI): m/z=542, 544 [M]⁺.

(13) 4-[(3-chloro-4-fluorophenyl)amino]-6-(2-{1-[(ethoxycarbonyl)methyl]piperidin-4-yl}ethylamino)pyrimido[5,4-d]pyrimidine

Melting point: 128° C.-130° C.; mass spectrum (EI): m/z=487, 489 [M]⁺.

(14) 4-[(3-chloro-4-fluorophenyl)amino]-6-{2-[4-(ethoxycarbonyl)piperidin-1-yl]ethylamino}pyrimido[5,4-d]pyrimidine

Melting point: 137° C.-139° C.; mass spectrum (EI): m/z=473, 475 [M]⁺.

(15) 4-[(3-chloro-4-fluorophenyl)amino]-6-(4-{N-[2-(methoxycarbonyl)ethyl]-N-methylamino}piperidin-1-yl)pyrimido[5,4-d]pyrimidine

R_{f} value: 0.15 (silica gel, petroleum ether/ethyl acetate/methanol=5:5:1); mass spectrum (EI): m/z=473, 475 [M]⁺.

(16) 4-[(3-chloro-4-fluorophenyl)amino]-6-(3-{4-[2-(methoxycarbonyl)ethyl]piperidin-1-yl}-pyrrolidin-1-yl)pyrimido[5,4-d]pyrimidine

Melting point: 166° C.-168° C.; mass spectrum (EI): m/z=513, 515 [M]⁺.

(17) 4-[(3-bromophenyl)amino]-6-({1-[(methoxycarbonyl)methyllpiperidin-4-yl}amino)pyrimido[5,4-d]pyrimidine

Melting point: 144° C.; mass spectrum (ESI⁺): m/z=472, 474 [M+H]⁺.

(18) 4-[(3-bromophenyl)amino]-6-(3-{N-[(methoxycarbonyl)methyl]-N-methylamino}propylamino)pyrimido[5,4-d]pyrimidine

R_{f} value: 0.35 (silica gel, cyclohexane/ethyl acetate/methanol=5:4:1); mass spectrum (ESI⁺): m/z=474,476 [M+H]+.

(19) 4-[(3-chloro-4-fluorophenyl)amino]-6-[4-({N-[(methoxycarbonyl)methyl]-N-methylamino}methyl)piperidin-1-yl]pyrimido[5,4-d]pyrimidine

R_{f} value: 0.88 (silica gel, methylene chloride/methanol/concentrated aqueous ammonia solution=90:10:0.1); mass spectrum (EI): m/z=473, 475 [M]⁺.

(20) 4-[(3-chloro-4-fluorophenyl)amino]-6-(4-{[2-(methoxycarbonyl)piperidin-1-yl]methyl}piperidin-1-yl)pyrimido[5,4-d]pyrimidine

R_{f} value: 0.73 (silica gel, petroleum ether/ethyl acetate/methanol=10:10:1); mass spectrum (ESI⁺): m/z=514, 516 [M+H]⁺.

(21) 4-[(3-chloro-4-fluorophenyl)amino]-6-(4-{[2-(methoxycarbonyl)pyrrolidin 1-yl]methyl}piperidin-1-yl)pyrimido[5,4-d]pyrimidine

Melting point: 151° C.-154° C.; mass spectrum (ESI): m/z=500, 502 [M+H]⁺.

(22) 4-[(3-chloro-4-fluorophenyl)amino]-6-[4-({4-[(ethoxycarbonyl)methyl]piperazin-1-yl}methyl)piperidin-1-yl]pyrimido[5,4-d]pyrimidine

Melting point: 145° C.-149° C.; mass spectrum (ESI⁺): m/z=543, 545 [M+H]⁺.

(23) 4-[(3-chlorophenyl)amino]-6-({1-[(methoxycarbonyl)methyl]piperidin-4-yl}amino)pyrimido[5,4-d]pyrimidine

Melting point: 129° C.; mass spectrum (EI): m/z=427, 429 [M]⁺.

(24) 4-[(3-methylphenyl)amino]-6-({1-[(methoxycarbonyl)methyl]piperidin-4-yl}amino)pyrimido[5,4-d]pyrimidine

Melting point: 164° C.; mass spectrum (EI): m/z=407 [M]⁺.

(25) (R)-4-[(1-phenylethyl)amino]-6-({1-[(methoxycarbonyl)methyl]piperidin-4-yl}amino)pyrimido[5,4-d]pyrimidine R_{f} value: 0.39 (silica gel, ethyl acetate/methanol=95:5); mass spectrum (EI): m/z=421 [M]⁺.

(26) 4-[(4-amino-3,5-dichlorophenyl)amino]-6-({1-[(methoxycarbonyl)methyl]piperidin-4-yl}amino)pyrimido[5,4-d]pyrimidine

Melting point: 218° C.; mass spectrum (EI): m/z=476, 478, 480 [M]⁺.

(27) 4-[(4-amino-3,5-dibromophenyl)amino]-6-({1-[(methoxycarbonyl)methyl]piperidin-4-yl}amino)pyrimido[5,4-d]pyrimidine

Melting point: 167° C.; mass spectrum (EI): m/z=564, 566, 568 [M]⁺.

(28) 4-[(indol-5-yl)amino]-6-({1-[(methoxycarbonyl)methyl]piperidin-4-yl}amino)pyrimido [5,4-d]pyrimidine

Melting point: 167° C.; mass spectrum (EI): m/z=432 [M]⁺.

### EXAMPLE 3

4-[(3-chloro-4-fluorophenyl)amino]-6-(2-{4-[(ethoxycarbonyl)methyl]piperazin-1-yl}ethylamino)pyrimido[5.4-d]pyrimidine

2.08 ml of triethylamine and 0.61 ml of ethyl bromoacetate are added to 2.01 g of 4-[(3-chloro-4-fluorophenyl)amino]-6-[2-(piperazin-1-yl)ethylamino]pyrimido[5,4-d]pyrimidine in 50 ml pyridine. The reaction mixture is stirred for two hours at ambient temperature. Then the reaction mixture is concentrated by evaporation, water is added, and the mixture is extracted with methylene chloride. The combined organic phases are dried over magnesium sulfate and concentrated by evaporation. The yellow crude product is purified by chromatography on an aluminium oxide column (activity III) with methylene chloride/ethanol (99:1). Yield: 1.97 g (81% of theory); melting point: 128° C.-129° C.; mass spectrum (ESI⁺): m/z=489 [M+H]⁺.

The following compounds are obtained analogously to Example 3:

(1) 4-[(3-chloro-4-fluorophenyl)amino]-6-(4-{N-[(ethoxycarbonyl)methyl]-N-methylamino}piperidin-1-yl)pyrimido[5,4-d]pyrimidine R_{f} value: 0.63 (silica gel, methylene chloride/methanol/concentrated aqueous ammonia solution=90:10:0.1); mass spectrum (EI): m/z=473, 475 [M]⁺.

(2) 4-[(3-chloro-4-fluorophenyl)amino]-6-[4-({[2-(methoxycarbonyl)ethyl]amino}methyl)piperidin-1-yl]pyrimido[5,4-d]pyrimidine

The reaction is carried out with methyl 3-bromopropionate. R_{f} value: 0.57 (silica gel, methylene chloride/methanol/concentrated aqueous ammonia solution=90:10:0.1); mass spectrum (EI): m/z=473, 475 [M]⁺.

(3) 4-[(3-chloro-4-fluorophenyl)amino] -6-[4-({[(methoxycarbonyl)methyl]amino } methyl)piperidin-1-yl]pyrimido[5 ,4-d]pyrimidine

R_{f} value: 0.66 (silica gel, methylene chloride/methanol/concentrated aqueous ammonia solution=90:10:0.1); mass spectrum (EI): m/z=459, 461 [M]⁺.

(4) 4-[(3-chloro-4-fluorophenyl)amino]-6-[(trans-4-{N,N-bis-[(ethoxycarbonyl)methyl]amino}cyclohex-1-yl)amino]pyrimido[5,4-d]pyrimidine

The reaction is carried out in acetonitrile with diisopropyl-ethylamine as the auxiliary base. Melting point: 155° C.-157° C.; mass spectrum (EI): m/z=559, 561 [M]⁺.

(5) 4-[(3-chloro-4-fluorophenyl)amino]-6-[(trans-4-{N,N-bis-[(methoxycarbonyl)methyl]amino }cyclohex-1-yl)amino]pyrimido [5,4-d]pyrimidine

The reaction is carried out in acetonitrile with diisopropyl-ethylamine as the auxiliary base. Melting point: 181° C.-184° C.; mass spectrum (ESI⁺): m/z=532, 534 [M+H]⁺.

(6) 4-[(3-chloro-4-fluorophenyl)amino]-6-[4-({[(ethoxycarbonyl)-methyl]amino}methyl)piperidin-1-yl]pyrimido[5,4-d]pyrimidine

The reaction is carried out in acetonitrile with diisopropyl-ethylamine as the auxiliary base. R_{f} value: 0.75 (silica gel, methylene chloride/methanol/concentrated aqueous ammonia solution 90:10:1); mass spectrum (EI): m/z=473, 475 [M]⁺.

(7) 4-[(3-chloro-4-fluorophenyl)amino]-6-[4-({N,N-bis[(ethoxy-carbonyl)methyl]amino}methyl)piperidin-1-yl]pyrimido[5,4-d]pyrimidine

The reaction is carried out in acetonitrile with diisopropyl-ethylamine as the auxiliary base. R_{f} value: 0.65 (silica gel, methylene chloride/methanol=95:5); mass spectrum (ESI⁺): m/z=560, 562 [M+H]⁺.

(8) 4-[(3-chloro-4-fluorophenyl)amino]-6-[4-({N,N-bis[(methoxy-carbonyl)methyl]amino}methyl)piperidin-1-yl]pyrimido[5,4-d]pyrimidine

The reaction is carried out in acetonitrile with diisopropyl-ethylamine as the auxiliary base. R_{f} value: 0.81 (silica gel, methylene chloride/methanol/concentrated aqueous ammonia solution 90:10:0.1); mass spectrum (ESI⁺): m/z=532, 534 [M+H]⁺.

(9) 4-[(3-chloro-4-fluorophenyl)amino]-6-[3-({N,N-bis[(methoxy-carbonyl)methyl]amino}methyl)piperidin-1-yl]pyrimido[5,4-d]pyrimidine

The reaction is carried out in acetonitrile with diisopropyl-ethylamine as the auxiliary base. R_{f} value: 0.83 (silica gel, methylene chloride/methanol/concentrated aqueous ammonia solution=90:10:0.1); mass spectrum (ESI): m/z=532, 534 [M+H]⁺.

(10) 4-[(3-chloro-4-fluorophenyl)amino]-6-[(trans-4-{[(methoxycarbonyl)methyl]amino}cyclohex-1-yl)amino]pyrimido[5,4-d]pyrimidine

The reaction is carried out in acetonitrile with diisopropyl-ethylamine as the auxiliary base. Melting point: 141° C.-143° C.; mass spectrum (EI): m/z=459, 461 [M]⁺. p0 (11) 4-[(3-chloro-4-fluorophenyl)amino]-6-[N-(trans-4-{[(methoxycarbonyl)methyl]amino}cyclohex-1-yl)-N-methylamino]pyrimido[5,4-d]pyrimidine

The reaction is carried out in acetonitrile with diisopropyl-ethylamine as the auxiliary base. Melting point: 169.5° C.-171.5° C.; mass spectrum (ESI⁺): m/z=474, 476 [M+H]⁺.

(12) 4-[(3-chloro-4-fluorophenyl)amino]-6-[N-(trans-4-{N,N′-bis[(methoxycarbonyl)methyl]amino}cyclohex-1-yl)-N-methylamino]pyrimido[5,4-d]pyrimidine

For method see Example 3(11). Melting point: 162-164° C.; mass spectrum (ESI⁺): m/z=546, 548 [M+H]⁺.

(13) 4-[(3-chloro-4-fluorophenyl)amino]-6-[3-({[(methoxycarbonyl)methyl]amino} methyl)piperidin-1-yl]pyrimido[5,4-d]pyrimidine

The reaction is carried out in acetonitrile with diisopropyl-ethylamine as the auxiliary base. R_{f} value: 0.76 (silica gel, methylene chloride/methanol/concentrated aqueous ammonia solution=90:10:0.1); mass spectrum (ESI⁺): m/z=460, 462 [M+H]⁺.

(14) 4-[(3-chloro-4-fluorophenyl)amino]-6-[N-(trans-4-{N′-[(methoxycarbonyl)methyl]-N′-methylaminol cyclohex-1-yl)-N-methylamino]pyrimido[5,4-d]pyrimidine

The reaction is carried out in acetonitrile with diisopropyl-ethylamine as the auxiliary base. Melting point: 137° C.-139.5° C.; mass spectrum (ESI⁺): m/z=488, 490 [M+H]⁺.

(15) 4-[(3-chloro-4-fluorophenyl)amino]-6-{4-methyl-4-[(methoxycarbonyl)methyl]aminopiperidin-1-yl}pyrimido[5,4-d]pyrimidine

The reaction is carried out in acetonitrile with diisopropyl-ethylamine as the auxiliary base. R_{f} value: 0.59 (silica gel, methylene chloride/methanol/concentrated aqueous ammonia solution=90:10:1); mass spectrum (ESI⁺): m/z=460, 462 [M+H]⁺.

(16) 4-[(3-bromophenyl)amino]-6-(1-[1,1-bis(methoxycarbonyl)methyl]piperidin-4-yl}amino)pyrimido[5,4-d]pyrimidine

The reaction is carried out with dimethyl bromomalonate in acetonitrile in the presence of diisopropyl-ethylamine as the auxiliary base. Melting point: 158-160° C.; mass spectrum (ESI⁺): m/z=530, 532 [M+H]⁺.

(17) 4-[(3-bromophenyl)amino]-6-({1-[(methoxycarbonyl)methyl]piperidin-3-yl}amino)pyrimido[5,4-d]pyrimidine

The reaction is carried out in acetonitrile with diisopropyl-ethylamine as the auxiliary base. Melting point: 113° C.; mass spectrum (ESI⁺): m/z=472, 474 [M+H]⁺.

(18) 4-[(3-chloro-4-fluorophenyl)amino]-6-(1-[1,1-bis(methoxycarbonyl)methyl]piperidin-4-yl}amino)pyrimido[5,4-d]pyrimidine

The reaction is carried out with dimethyl bromomalonate in acetonitrile in the presence of diisopropyl-ethylamine as the auxiliary base. Melting point: 192° C.-193° C.; mass spectrum (ESI⁺): m/z=504, 506 [M+H]⁺.

### EXAMPLE 4

4-[(3-chloro-4-fluorophenyl)amino]-6-(2-{4-[(diethoxyphosphoryl)methyl]piperazin-1-yl}ethylamino)pyrimido[5.4-d]pyrimidine

A suspension of 500 mg of 4-[(3-chloro-4-fluorophenyl)amino]-6-[2-(piperazin-1-yl)ethylamino]pyrimido[5,4-d]pyrimidine in 15 ml dioxane is heated to 95° C.-100° C. with stirring until the solid is substantially dissolved. Then, initially 100 μl of 37% formaldehyde solution and 190 μl of diethylphosphite are added with heating. The reaction mixture is stirred for about 4 hours at 100° C. For working up, the reaction mixture is concentrated by evaporation, the residue is stirred with a little ice-cold water and extracted with methylene chloride. The combined organic phases are dried over sodium sulfate and concentrated by evaporation. The brownish-yellow crude product is purified by chromatography on an aluminium oxide column (activity III) with methylene chloride/methanol (98.5:1.5). Yield: 250 mg (36% of theory); R_{f} value: 0.70 (silica gel, methylene chloride/methanol/concentrated aqueous ammonia solution=9:1:0.01); mass spectrum (ESI⁻): m/z=551, 553 [M-H]⁻⁻

The following compounds are obtained analogously to Example 4:

(1) 4-[(3-bromophenyl)amino]-6-({1-[(diethoxyphosphoryl)methyl]piperidin-4-yl}amino)pyrimido[5,4-d]pyrimidine

R_{f} value: 0.36 (silica gel, methylene chloride/methanol/concentrated aqueous ammonia solution=90:10:0.5); mass spectrum (EJ): m/z=549, 551 [M]⁺.

(2) 4-[(3-bromophenyl)amino]-6-[(1-{[(ethoxy)(methyl)phosphoryl]methyl}piperidin-4-yl)amino]pyrimido [5,4-d]pyrimidine

Reaction with diethoxymethylphosphine in tetrahydrofuran. R_{f} value: 0.25 (silica gel, methylene chloride/methanol/concentrated aqueous ammonia solution=90:10:1); mass spectrum (EI): m/z=519, 521 [M]⁺.

### EXAMPLE 5

4-[(3-chloro-4-fluorophenyl)amino]-6-[4-amino-4-(methoxycarbonyl)piperidin-1-yl]pyrimido[5.4-d]pyrimidine

A suspension of 720 mg 4-[(3-chloro-4-fluorophenyl)amino]-6-{4-[(tert-butyloxycarbonyl)amino]-4-(methoxycarbonyl)piperidin-1-yl}pyrimido[5,4-d]pyrimidine in 10 ml methylene chloride is mixed with 2 ml trifluoroacetic acid with stirring. The solution formed with the release of gas is left to stand overnight and then evaporated to dryness. The residue is taken up in methylene chloride, washed with dilute potassium carbonate solution and water, and dried over magnesium sulfate. The solvent is distilled off and the yellow resin remaining is stirred with a little methanol. The yellow precipitate is suction filtered, washed with a little cold methanol, and dried in the desiccator. Yield: 565 mg (97% of theory); melting point: 182-184° C.; mass spectrum (ESI⁺): m/z=432, 434 [M+H]⁺.

### EXAMPLE 6

4-[(3-chloro-4-fluorophenyl)aminol-6-[4-({INN-bis[2-(methoxycarbonyl)ethyl]amino}methyl)piperidin-1-yl]pyrimido[5.4-d]pyrimidine

0.73 ml methyl acrylate is added to 1.00 g of 4-[(3-chloro-4-fluorophenyl)amino]-6-(4-aminomethylpiperidin-1-yl)pyrimido[5,4-d]pyrimidine in 25 ml methanol. The reaction mixture is refluxed for four hours, then another 0.35 ml of methyl acrylate is added. After another five hours under reflux, the reaction is almost complete and the mixture is concentrated by evaporation. The orange-yellow crude product is purified by chromatography on a silica gel column with petroleum ether/ethyl acetate/methanol (1:1:0.1) as eluant. Yield: 1.02 g (71% of theory); melting point: 113° C.-118° C.; mass spectrum (ESI⁺): m/z=560, 562 [M+H]⁺.

The following compounds are obtained analogously to Example 6:

(1) 4-[(3-chloro-4-fluorophenyl)amino]-6-[3-({N,N-bis[2-(methoxycarbonyl)ethyl]amino}methyl)piperidin-1-yl]pyrimido-[5,4-d]pyrimidine

R_{f} value: 0.90 (silica gel, methylene chloride/methanol/concentrated aqueous ammonia solution=90:10:0.1); mass spectrum (ESI⁺): m/z=560, 562 [M+H]⁺.

(2) 4-[(3-chloro-4-fluorophenyl)amino]-6-[3-({[2-(methoxycarbonyl)ethyl]amino}methyl)piperidin-1-yl]pyrimido[5,4-d]pyrimidine

Only 1.5 equivalents of methyl acrylate are used. R_{f} value: 0.60 (silica gel, methylene chloride/methanol/concentrated aqueous ammonia solution=90:10:0.1); mass spectrum (ESI⁺): m/z=474, 476 [M+H]⁺.

(3) 4-[(3-chloro-4-fluorophenyl)amino]-6-{4-methyl-4-[2-(methoxycarbonyl)ethyl]aminopiperidin-1-yl}pyrimido[5,4-d]pyrimidine

Only 1.4 equivalents of methyl acrylate are used). Melting point: 134-135° C.; mass spectrum (ESI⁺): m/z=474, 476 [M+H]⁺.

(4) 4-[(3-bromophenyl)amino]-6-({1-[1,2-bis(methoxycarbonyl)ethyl]piperidin-4-yl}amino)pyrimido[5,4-d]pyrimidine

The reaction is carried out with dimethyl maleate in dioxane. Melting point: 193° C.; mass spectrum (ESI⁻): m/z=542, 544 [M-H]⁻.

(5) 4-[(3-bromophenyl)amino]-6-[(1-{1-[(ethoxycarbonyl)methyl]-2-(ethoxycarbonyl)ethyl}piperidin-4-yl)amino]pyrimido-[5,4-d]pyrimidine

The reaction is carried out with diethyl glutaconate in dioxane. Melting point: 132° C.; mass spectrum (ESI⁺): m/z=586, 588 [M+H]⁺.

### EXAMPLE 7

4-[(3-chloro-4-fluorophenyl)amino]-6-{[trans-4-(2-oxomorpholin-4-yl)cvclohex-1-yl]amino}pyrimido[5,4-d]pyrimidine

0.61 ml of diisopropylethylamine and 0.39 ml of ethyl bromoacetate is added to 970 mg of 4-[(3-chloro-4-fluorophenyl)amino]-6-({trans-4-[(2-hydroxyethyl)amino]cyclohex-1-yl}amino)pyrimido[5,4-d]pyrimidine in 5 ml dimethylformamide at ambient temperature. The suspension is briefly heated to 50° C. in a water bath until a clear solution is formed. Then the reaction mixture is stirred for a further three hours at ambient temperature. For working up, the mixture is combined with ice-cold water. The phases are separated and the aqueous phase is extracted with ethyl acetate. The combined organic phases are washed with water and saturated sodium chloride solution, dried over magnesium sulfate, and concentrated by evaporation. The crude product is purified by chromatography on a silica gel column with methylene chloride/methanol (98.5:1.5 to 97:3) as eluant. Product is obtained exclusively as a yellow crystalline solid. Yield: 466 mg (44% of theory); melting point: 213-223° C.; mass spectrum (ESI⁺): m/z=472, 474 [M+H]⁺.

The following compounds are obtained analogously to Example 7:

(1) 4-[(3-chloro-4-fluorophenyl)amino]-6-{4-[(2-oxomorpholin-4-yl)methyl]piperidin 1-yl}pyrimido[5,4-d]pyrimidine

The reaction is carried out in acetonitrile as solvent, producing predominantly non-cyclized product which is cyclized to form the lactone by heating with a little p-toluenesulfonic acid in toluene. Melting point: 202-204° C.; mass spectrum (ESI⁺): m/z=472, 474 [M+H]⁺.

### EXAMPLE 8

4-[(3-chloro-4-fluorophenyl)amino]-6-[(trans-4-{[(2-{N-[(methoxycarbonyl)methyl]-N-methylamino}ethyl)sulfonyl]amino}cyclohex-1-yl)amino]pyrimido[5.4-d]pyrimidine

0.21 ml Diisopropylethylamine and 176 mg of sarcosine methyl ester hydrochloride are added to 195 mg of 4-[(3-chloro-4-fluorophenyl)amino]-6-({trans-4-[(vinylsulfonyl)amino]cyclohex-1-yl}amino)pyrimido[5,4-d]pyrimidine in 10 ml methanol at ambient temperature. The reaction mixture is refluxed for about 25 hours. After the reaction has ended the mixture is concentrated by evaporation. Since the product is obviously partly in the form of the free acid the residue is again dissolved in methanol, cooled under a nitrogen atmosphere in a bath of acetone/dry ice, and combined with 0.2 ml of thionyl chloride. After heating to ambient temperature the solvent is distilled off in vacuo, the residue is dissolved in methylene chloride/methanol, washed with dilute sodium carbonate solution, dried over magnesium sulfate, and concentrated by evaporation. The brownish crude product is purified by chromatography on a silica gel column with methylene chloride/methanol (98:2). Yield: 51 mg (22% of theory); melting point: 171-174° C.; mass spectrum (ESI⁺): m/z=581, 583 [M+H]⁺.

The following compound is obtained analogously to Example 8:

(1) 4-[(3-chloro-4-fluorophenyl)amino]-6-{[trans-4-({[2-(2-oxomorpholin-4-yl)ethyl]sulfonyl}amino)cyclohex-1-yl]amino}pyrimido[5,4-d]pyrimidine

By reaction with ethyl (2-hydroxyethylamino)acetate hydrochloride in ethanol with no subsequent re-esterification as described in Example 8. R_{f} value: 0.39 (silica gel, methylene chloride/methanol=95:5); mass spectrum (EI): m/z=578, 580 [M]⁺.

The following compounds may also be obtained analogously to the preceding Examples and other methods known from the literature:

(1) 4-[(3-methylphenyl)amino]-6-({1-[(methoxycarbonyl)methyl]piperidin-4-yl}amino)pyrimido[5,4-d]pyrimidine

(2) 4-[(3-chlorophenyl)amino]-6-({1-[(methoxycarbonyl)methyl]piperidin-4-yl}amino)pyrimido[5,4-d]pyrimidine

(3) 4-[(3-ethynylphenyl)amino]-6-({1-[(methoxycarbonyl)methyl]piperidin-4-yl}amino)pyrimido[5,4-d]pyrimidine

(4) 4-[(3-trifluoromethylphenyl)amino]-6-({1-[(methoxycarbonyl)methyl]piperidin-4-yl}amino)pyrimido[5,4-d]pyrimidine

(5) 4-[(3-bromophenyl)amino]-6-({1-[(methoxycarbonyl)methyl]piperidin-4-yl}amino)pyrimido[5,4-d]pyrimidine

(6) 4-[(4-amino-3,5-dibromo-phenyl)amino]-6-({1-[(methoxycarbonyl)methyl]piperidin-4-yl}amino)pyrimido[5,4-d]pyrimidine

(7) 4-[(4-amino-3,5-dichlor-phenyl)amino]-6-({1-[(methoxycarbonyl)methyl]piperidin-4-yl}amino)pyrimido[5,4-d]pyrimidine

(8) 4-[(indol-5-yl)amino]-6-({1-[(methoxycarbonyl)methyl]piperidin-4-yl}amino)pyrimido[5,4-d]pyrimidine

(9) 4-[(3-bromophenyl)amino]-6-(N-{1-[(methoxycarbonyl)methyl]piperidin-4-yl}-N-methylamino)pyrimido[5,4-d]pyrimidine

(10) 4-[(3-bromophenyl)amino]-6-({1-[(methoxycarbonyl)ethyl]piperidin-4-yl}amino)pyrimido [5,4-d]pyrimidine

(11) 4-[(3-chlorophenyl)amino]-6-({1-[(methoxycarbonyl)ethyl]piperidin-4-y}amino)pyrimido[5,4-d]pyrimidine

(12) 4-[(3-chloro-4-fluorophenyl)amino]-6-({1-[(methoxycarbonyl)ethyl]piperidin-4-yl}amino)pyrimido[5,4-d]pyrimidine

(13) 4-[(3-methylphenyl)amino]-6-({1-[(methoxycarbonyl)ethyl]piperidin-4-yl}amino)pyrimido[5,4-d]pyrimidine

(14) 4-[(3-ethynylphenyl) amino]-6-({1-[(methoxycarbonyl)ethyl]piperidin-4-yl} amino)pyrimido[5,4-d]pyrimidine

(15) 4-[(3-chlorophenyl)amino]-6-({1-[1,2-bis(methoxycarbonyl)ethyl]piperidin-4-yl}amino)pyrimido[5,4-d]pyrimidine

(16) 4-[(3-chlorophenyl)amino]-6-[(1-{1-[(methoxycarbonyl)methyl]-2-(methoxycarbonyl)ethyl}piperidin-4-yl)amino]pyrimido[5,4-d]pyrimidine

(17) 4-[(3-chlorophenyl)amino]-6-[(1-{1-[(ethoxycarbonyl)methyl]-2-(ethoxycarbonyl)ethyllpiperidin-4-yl)amino]pyrimido[5,4-d]pyrimidine

(18) 4-[(3-chlorophenyl)amino]-6-({1-[1,2-bis(ethoxycarbonyl)ethyl]piperidin-4-yl}amino)pyrimido[5,4-d]pyrimidine

(19) 4-[(3-chlorophenyl)amino]-6-({1-[(diethoxyphosphoryl)methyl]piperidin-4-yl}amino)pyrimido[5,4-d]pyrimidine

(20) 4-[(3-chlorophenyl)amino]-6-({1-[(dimethoxyphosphoryl)methyl]piperidin-4-yl}amino)pyrimido[5,4-d]pyrimidine

(21) 4-[(3-chlorophenyl)amino]-6-[(1-{[(methoxy)(methyl)phosphoryl]methyl}piperidin-4-yl)amino]pyrimido[5,4-d]pyrimidine

(22) 4-[(3-chlorophenyl)amino]-6-[(1-{[(ethoxy)(methyl)phosphoryl]methyl}piperidin-4-yl)amino]pyrimido[5,4-d]pyrimidine

(23) 4-[(3-chlorophenyl)amino]-6-({1-[(hexyloxycarbonyl)methyl]piperidin-4-yl}amino)pyrimido [5,4-d]pyrimidine

(24) 4-[(3-chlorophenyl)amino]-6-(2-{N-[(methoxycarbonyl)methyl]-N-methylamino}ethylamino)pyrimido[5,4-d]pyrimidine

(25) 4-[(3-chlorophenyl)amino]-6-(3-{N-[(methoxycarbonyl)methyl]-N-methylamino}propylamino)pyrimido[5,4-d]pyrimidine

(26) 4-[(3-chlorophenyl)amino]-6-(4-{N-[(methoxycarbonyl)methyl]-N-methylamino}butylamino)pyrimido [5,4-d]pyrimidine

(27) 4-[(3-chlorophenyl)amino]-6-(3-{NN-bis[(methoxycarbonyl)-methyl]amino}propylamino)pyrimido[5,4-d]pyrimidine

(28) 4-[(3-chlorophenyl)amino]-6-({-[(methoxycarbonyl)methyl]piperidin-3-yl}amino)pyrimido[5,4-d]pyrimidine

(29) 4-[(3-bromophenyl)amino]-6-({1-[(methoxycarbonyl)methyl]piperidin-3-yl}amino)pyrimido[5,4-d]pyrimidine

(30) 4-[(3-methylphenyl)amino]-6-({1-[(methoxycarbonyl)methyl]piperidin-3-yl}amino)pyrimido[5,4-d]pyrimidine

(31) 4-[(3-ethynylphenyl)amino]-6-({1-[(methoxycarbonyl)methyl]piperidin-3-yl}amino)pyrimido[5,4-d]pyrimidine

(32) 4-[(3-chlorophenyl)amino]-6-({1-[(methoxycarbonyl)methyl]-azepan-4-yl}amino)pyrimido [5,4-d]pyrimidine

(33) 4-[(3-bromophenyl)amino]-6-({1-[(methoxycarbonyl)methyl]-azepan-4-yl }amino)pyrimido[5,4-d]pyrimidine

(34) 4-[(3-methylphenyl)amino]-6-({1-[(methoxycarbonyl)methyl]-azepan-4-yl}amino)pyrimido[5,4-d]pyrimidine

(35) 4-[(3-ethynylphenyl)amino]-6-({1-[(methoxycarbonyl)methyl]-azepan-4-yl}amino)pyrimido[5,4-d]pyrimidine

(36) 4-[(3-chlorophenyl)amino]-6-(4-{N-[(ethoxycarbonyl)methyl]-N-methylamino}piperidin-1-yl)pyrimido[5,4-d]pyrimidine

(37) 4-[(3-chlorophenyl)amino]-6-(4-{N-[(methoxycarbonyl)methyl]-N-methylamino}piperidin-1-yl)pyrimido[5,4-d]pyrimidine

(38) 4-[(3-chlorophenyl)amino]-6-(4-[(methoxycarbonyl)methyl]aminopiperidin-1-yl)pyrimido[5,4-d]pyrimidine

(39) 4-[(3-chlorophenyl)amino]-6-(4-{NN-bis[(methoxycarbonyl)methyl]amino}piperidin-1-yl)pyrimido[5,4-d]pyrimidine

(40) 4-[(3-chlorophenyl)amino]-6-(4-{N-[(dimethoxyphosphoryl)methyl]-N-methylamino}piperidin-1-yl)pyrimido[5,4-d]pyrimidine

(41) 4-[(3-chlorophenyl)amino]-6-[4-(N-{[(ethoxy)(methyl)phosphoryl]methyl}-N-methylamino)piperidin-1-yl]pyrimido[5,4-d]pyrimidine

(42) 4-[(3-chlorophenyl)amino]-6-[4-({N-[(methoxycarbonyl)methyl]-N-methylamino}methyl)piperidin-1-yl]pyrimido[5,4-d]pyrimidine

(43) 4-[(3-bromophenyl)amino]-6-[4-({N-[(methoxycarbonyl)methyl]-N-methylamino}methyl)piperidin-1-yl]pyrimido[5,4-d]pyrimidine

(44) 4-[(3-methylphenyl)amino]-6-[4-({N-[(methoxycarbonyl)methyl]-N-methylamino}methyl)piperidin-1-yl]pyrimido[5,4-d]pyrimidine

(45) 4-[(3-ethynylphenyl)amino]-6-[4-({N-[(methoxycarbonyl)methyl]-N-methylamino}methyl)piperidin-1-yl]pyrimido [5,4-d]pyrimidine

(46) 4-[(3-chlorophenyl)amino]-6-[4-({N-[(ethoxycarbonyl)methyl]-N-methylamino}methyl)piperidin-1-yl]pyrimido[5,4-d]pyrimidine

(47) 4-[(3-chlorophenyl)amino]-6-[4-({[(ethoxycarbonyl)methyl]amino}methyl)piperidin 1-yl]pyrimido[5,4-d]pyrimidine

(48) 4-[(3-chlorophenyl)amino]-6-[4-({N,N-bis[(ethoxycarbonyl)-methyl]amino}methyl)piperidin-1-yl]pyrimido[5,4-d]pyrimidine

(49) 4-[(3-chlorophenyl)amino]-6-[4-({N-[(dimethoxyphosphoryl)methyl]-N-methylamino}methyl)piperidin-1-yl]pyrimido[5,4-d]pyrimidine

(50) 4-[(3-chlorophenyl)amino]-6-[4-({N-[(diethoxyphosphoryl)methyl]-N-methylamino}methyl)piperidin-1-yl]pyrimido[5,4-d]pyrimidine

(51) 4-[(3-chlorophenyl)amino]-6-[4-(N-{[(ethoxy)(methyl)phosphoryl]methyl}-N-methylamino)methyl]piperidin-1-yl]pyrimido[5,4-d]pyrimidine

(52) 4-[(3-chlorophenyl)amino]-6-(2-{1-[(methoxycarbonyl)methyl]piperidin-4-yl}ethylamino)pyrimido[5,4-d]pyrimidine

(53) 4-[(3-bromophenyl)amino]-6-(2-{1-[(methoxycarbonyl)methyl]piperidin-4-yl}ethylamino)pyrimido[5,4-d]pyrimidine

(54) 4-[(3-methylphenyl)amino]-6-(2-{1-[(methoxycarbonyl)methyl]piperidin-4-yl} ethylamino)pyrimido[5,4-d]pyrimidine

(55) 4-[(3-ethynylphenyl)amino] -6-(2-{1-[(methoxycarbonyl)methyl]piperidin-4-yl }ethylamino)pyrimido[5,4-d]pyrimidine

(56) 4-[(3-chlorophenyl)amino]-6-(2-{4-[(methoxycarbonyl)methyl]piperazin 1-yl}ethylamino)pyrimido[5,4-d]pyrimidine

(57) 4-[(3-bromophenyl)amino]-6-(2-{4-[(methoxycarbonyl)methyl]piperazin-1-yl}ethylamino)pyrimido[5,4-d]pyrimidine

(58) 4-[(3-ethynylphenyl)amino]-6-(2-{4-[(methoxycarbonyl)methyl]piperazin-1-yl}ethylamino)pyrimido[5,4-d]pyrimidine

(59) 4-[(3-methylphenyl)amino]-6-(2-{4-[(methoxycarbonyl)methyl]piperazin-1-yl}ethylamino)pyrimido[5,4-d]pyrimidine

(60) 4-[(3-chlorophenyl)amino]-6-(2-{⁴-[(dimethoxyphosphoryl)methyl]piperazin-1-yl}ethylamino)pyrimido[5,4-d]pyrimidine

(61) 4-[(3-chlorophenyl)amino]-6-(2-{1-[(dimethoxyphosphoryl)methyl]piperidin-4-yl}ethylamino)pyrimido [5,4-d]pyrimidine

(62) 4-[(3-chlorophenyl)amino]-6-[2-(4-{[(ethoxy)(methyl)phosphoryl]methyl}piperazin-1-yl)ethylamino]pyrimido[5,4-d]pyrimidine

(63) 4-[(3-chlorophenyl)amino]-6-[2-(1-{[(ethoxy)(methyl)phosphoryl]methyl}piperidin-4-yl)ethylamino]pyrimido[5,4-d]pyrimidine

(64) 4-[(3-chlorophenyl)amino]-6-(3-{1-[(methoxycarbonyl)methyl]piperidin-4-yl}propylamino)pyrimido[5,4-d]pyrimidine

(65) 4-[(3-chlorophenyl)amino]-6-(3-{4-[(methoxycarbonyl)methyl]piperazin-1-yl}propylamino)pyrimido[5,4-d]pyrimidine

(66) 4-[(3-bromophenyl)amino]-6-(3-{4-[(methoxycarbonyl)methyl]piperazin-1-yl}propylamino)pyrimido[5,4-d]pyrimidine

(67) 4-[(3-methylphenyl)amino]-6-(3-{4-[(methoxycarbonyl)methyl]piperazin-1-yl}propylamino)pyrimido[5,4-d]pyrimidine

(68) 4-[(3-ethynylphenyl)amino]-6-(3-{4-[(methoxycarbonyl)methyl]piperazin-1-yl}propylamino)pyrimido[5,4-d]pyrimidine

(69) 4-[(3-chloro-4-fluorophenyl)amino]-6-(3-{4-[(methoxycarbonyl)methyl]piperazin-1-yl}propylamino)pyrimido[5,4-d]pyrimidine

(70) 4-[(3-chlorophenyl)amino]-6-({1-[(ethoxycarbonyl)methyl]piperidin-4-yl}methylamino)pyrimido[5,4-d]pyrimidine

(71) 4-[(3-bromophenyl)amino]-6-({1-[(ethoxycarbonyl)methyl]piperidin-4-yl}methylamino)pyrimido[5,4-d]pyrimidine

(72) 4-[(3-methylphenyl)amino]-6-({1-[(ethoxycarbonyl)methyl]piperidin-4-yl}methylamino)pyrimido[5,4-d]pyrimidine

(73) 4-[(3-ethynylphenyl)amino]-6-({1-[(ethoxycarbonyl)methyl]piperidin-4-yl}methylamino)pyrimido[5,4-d]pyrimidine

(74) 4-[(3-chlorophenyl)amino]-6-{[4-({N-[(ethoxycarbonyl)methyl]-N-methylamino}methyl)cyclohex-1-yl]methylamino}pyrimido[5,4-d]pyrimidine

(75) 4-[(3-chlorophenyl)amino]-6-[(4-{N-[(ethoxycarbonyl)methyl]-N-methylamino}cyclohex-1-yl)methylamino]pyrimido[5,4-d]pyrimidine

(76) 4-[(3-chlorophenyl)amino]-6-[(4-{[(ethoxycarbonyl)methyl]amino}cyclohex-1-yl)amino]pyrimido[5,4-d]pyrimidine

(77) 4-[(3-chloro-4-fluorophenyl)amino]-6-[(4-{[(ethoxycarbonyl)methyl]amino}cyclohex-1-yl)amino]pyrimido[5,4-d]pyrimidine

(78) 4-[(3-methylphenyl)amino]-6-[(4-{[(ethoxycarbonyl)methyl]amino}cyclohex-1-yl)amino]pyrimido[5,4-d]pyrimidine

(79) 4-[(3-bromophenyl)amino]-6-[(4-{[(ethoxycarbonyl)methyl]amino}cyclohex-1-yl)amino]pyrimido[5,4-d]pyrimidine

(80) 4-[3-(ethynylphenyl)amino]-6-[(4-{[(ethoxycarbonyl)methyl]amino}cyclohex-1-yl)amino]pyrimido[5,4-d]pyrimidine

(81) 4-[(3-chlorophenyl)amino]-6-{[4-({N-[(ethoxycarbonyl)methyl]-N-methylamino}methyl)cyclohex-1-yl]amino}pyrimido[5,4-d]pyrimidine (82) 4-[(3-chlorophenyl)amino]-6-({4-[(3-{N-[(ethoxycarbonyl)methyl]-N-methylamino}propyl)aminocarbonyl]cyclohex-1-yl} amino)pyrimido[5,4-d]pyrimidine

(83) 4-[(3-chlorophenyl)amino]-6-{[4-({1-[(methoxycarbonyl)methyl]piperidin-4-yl}aminocarbonyl)cyclohex-1-yl]amino}pyrimido[5,4-d]pyrimidine

(84) 4-[(3-chlorophenyl)amino]-6-{[4-({4-[(methoxycarbonyl)methyl]piperazin-1-yl}carbonyl)cyclohex-1-yl]amino}pyrimido[5,4-d]pyrimidine

(85) 4-[(3-chlorophenyl)amino]-6-[4-(2-{N-[(ethoxycarbonyl)methyl]-N-methylamino}ethyl)piperazin-1-yl]pyrimido[5,4-d]pyrimidine

(86) 4-[(3-chlorophenyl)amino]-6-(4-{1-[(methoxycarbonyl)methyl]piperidin-4-yl}piperidin-1-yl)pyrimido[5,4-d]pyrimidine

(87) 4-[(3-chlorophenyl)amino]-6-{7-[(methoxycarbonyl)methyl]-2,7-diaza-spiro[4.4]non-2-yl}pyrimido[5,4-d]pyrimidine

(88) 4-[(3-chlorophenyl)amino]-6-[(1-{1-[(methoxycarbonyl)methyl]piperidin-4-yl}piperidin-4-yl)amino]pyrimido[5,4-d]pyrimidine

(89) 4-[(3-chlorophenyl)amino]-6-({1-[(methoxycarbonyl)methyl]piperidin-4-yl}amino)pyrido[3,4-d]pyrimidine

(90) 4-[(3-bromophenyl)amino]-6-({1-[(methoxycarbonyl)methyl]piperidin-4-yl}amino)pyrido[3,4-d]pyrimidine

(91) 4-[(3-methylphenyl)amino]-6-({1-[(methoxycarbonyl)methyl]piperidin-4-yl}amino)pyrido[3,4-d]pyrimidine

(92) 4-[(3-ethynylphenyl)amino]-6-({1-[(methoxycarbonyl)methyl]piperidin-4-yl}amino)pyrido[3,4-d]pyrimidine

(93) 4-[(3-chloro-4-fluorophenyl)amino]-6-({1-[(methoxycarbonyl)methyl]piperidin-4-yl}amino)pyrido[3,4-d]pyrimidine

(94) 4-[(3-chlorophenyl)amino]-6-({1-[(methoxycarbonyl)methyl]piperidin-4-yl}amino)pyrido[3,2-d]pyrimidine

(95) 4-[(3-bromophenyl)amino]-6-({1-[(methoxycarbonyl)methyl]piperidin-4-yl}amino)pyrido[3,2-d]pyrimidine

(96) 4-[(3-methylphenyl)amino]-6-({1-[(methoxycarbonyl)methyl]piperidin-4-yl}amino)pyrido[3,2-d]pyrimidine

(97) 4-[(3-ethynylphenyl)amino]-6-({1-[(methoxycarbonyl)methyl]piperidin-4-yl}amino)pyrido[3,2-d]pyrimidine

(98) 4-[(3-chlorophenyl)amino]-7-({1-[(methoxycarbonyl)methyl]piperidin-4-yl}amino)pyrido[4,3-d]pyrimidine

(99) 4-[(3-chlorophenyl)amino]-7-({1-[(methoxycarbonyl)methyl]piperidin-4-yl}amino)pyrimido[4,5-d]pyrimidine

(100) 4-[(3-chlorophenyl)amino]-7-({1-[(methoxycarbonyl)methyl]piperidin-4-yl}amino)pyrido[2,3-d]pyrimidine

(101) 4-[(3-chlorophenyl)amino]-6-[4-amino-4-(methoxycarbonyl)piperidin-1-yl]pyrimido[5,4-d]pyrimidine

(102) 4-[(3-bromophenyl)amino]-6-[4-amino-4-(methoxycarbonyl)piperidin-1-yl]pyrimido [5,4-d]pyrimidine

(103) 4-[(3-methylphenyl)amino]-6-[4-amino-4-(methoxycarbonyl)piperidin-1-yl]pyrimido[5,4-d]pyrimidine

(104) 4-[(3-ethynylphenyl)amino]-6-[4-amino-4-(methoxycarbonyl)piperidin-1-yl]pyrimido[5,4-d]pyrimidine

(105) 4-[(3-chloro-4-luorophenyl)amino]-6-[4-amino-4-(methoxycarbonyl)piperidin-1-yl]pyrimido[5,4-d]pyrimidine

(106) 4-[(1-phenylethyl)amino]-6-({1-[(methoxycarbonyl)methyl]piperidin-4-yl}amino)pyrimido[5,4-d]pyrimidine

(107) 4-[(3-chlorophenyl)amino]-6-{[4-(2-oxomorpholin-4-yl)cyclohex-1-yl]amino}pyrimido[5,4-d]pyrimidine

(108) 4-[(3-chlorophenyl)amino]-6-{4-[(2-oxomorpholin-4-yl)methyl]piperidin-1-yl}pyrimido[5,4-d]pyrimidine

(109) 4-[(3-chlorophenyl)amino]-6-{[2-(2-oxomorpholin-4-yl)ethyl]aminopyrimido[5,4-d]pyrimidine

Example 9: Coated Tablets Containing 75 mg of Active Substance

| Component | Amount per tablet core (mg) |
|---|---|
| active substance | 75 |
| calcium phosphate | 93.0 |
| corn starch | 35.5 |
| polyvinylpyrrolidone | 10.0 |
| hydroxypropylmethylcellulose | 15.0 |
| magnesium stearate | 1.5 |
| TOTAL | 230.0 |

Preparation:

The active substance is mixed with calcium phosphate, corn starch, polyvinylpyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape. Weight of core: 230 mg; die: 9 mm, convex. The tablet cores thus produced are coated with a film consisting essentially of hydroxypropylmethylcellulose. The finished film-coated tablets are polished with beeswax. Weight of coated tablet: 245 mg.

Example 10: Tablets Containing 100 mg of Active Substance

| Component | Amount per tablet (mg) |
|---|---|
| active substance | 100.0 |
| lactose | 80.0 |
| corn starch | 34.0 |
| polyvinylpyrrolidone | 4.0 |
| magnesium stearate | 2.0 |
| TOTAL | 220.0 |

Preparation:

The active substance, lactose, and starch are mixed together and uniformly moistened with an aqueous solution of the polyvinylpyrrolidone. After the moist composition has been screened (2.0 mm mesh size) and dried in a rack-type drier at 50° C., it is screened again (1.5 mm mesh size) and the lubricant is added. The finished mixture is compressed to form tablets. Weight of tablet: 220 mg; diameter: 10 mm, biplanar, facetted on both sides and notched on one side.

Example 11: Tablets Containing 150 mg of Active Substance

| Component | Amount per tablet (mg) |
|---|---|
| active substance | 150.0 |
| powdered lactose | 89.0 |
| corn starch | 40.0 |
| colloidal silica | 10.0 |
| polyvinylpyrrolidone | 10.0 |
| magnesium stearate | 1.0 |
| TOTAL | 300.0 |

Preparation:

The active substance mixed with lactose, corn starch, and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture. Weight of tablet: 300 mg; die: 10 mm, flat.

Example 12: Hard Gelatine Capsules Containing 150 mg of
Active Substance

| Component | Amount per capsule (mg) |
|---|---|
| active substance | 150.0 |
| corn starch (dried) | approx. 80.0 |
| lactose (powdered) | approx. 87.0 |
| magnesium stearate | 3.0 |
| TOTAL | 320.0 |

Preparation:

The active substance is mixed with the excipients, passed through a screen with a mesh size of 0.75 mm and homogeneously mixed using a suitable apparatus. The finished mixture is packed into size 1 hard gelatine capsules. Capsule filling: approx. 320 mg; capsule shell: size 1 hard gelatine capsule.

Example 13: Suppositories Containing 150 mg of Active Substance

| Component | Amount per suppository (mg) |
|---|---|
| active substance | 150.0 |
| polyethyleneglycol 1500 | 550.0 |
| polyethyleneglycol 6000 | 460.0 |
| polyoxyethylene sorbitan monostearate | 840.0 |
| TOTAL | 2000.0 |

Preparation:

After the suppository mass has been melted the active substance is homogeneously distributed therein and the melt is poured into chilled molds.

Example 14: Suspension Containing 50 mg of Active Substance/5 ml

| | |
|---|---|
| Component | Amount/100 ml suspension |

| | | |
|---|---|---|
| active substance | 1.0 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavoring | 0.30 | g |
| distilled water | ad 100 | ml |

Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavoring have been added and dissolved, the suspension is evacuated with stirring to eliminate air. 5 ml of suspension contains 50 mg of active substance.

Example 15: Ampoules Containing 10 mg of Active Substance

| Component | Amount |
|---|---|
| active substance | 10.0 mg |
| 0.01N hydrochloric acid | q.s. |
| double-distilled water | ad 2.0 ml |

Preparation:

The active substance is dissolved in the necessary amount of 0.01 N HCl, made isotonic with common salt, filtered sterile, and transferred into 2 ml ampoules.

Example 16: Ampoules Containing 50 mg of Active Substance

| Component | Amount |
|---|---|
| active substance | 50.0 mg |
| 0.01N hydrochloric acid | q.s. |
| double-distilled water | ad 10.0 ml |

Preparation:

The active substance is dissolved in the necessary amount of 0.01 N HCl, made isotonic with common salt, filtered sterile, and transferred into 10 ml ampoules.

Example 17: Capsules for Powder Inhalation Containing 5 mg of
Active Substance

| Component | Amount per capsule (mg) |
|---|---|
| active substance | 5.0 |
| lactose for inhalation | 15.0 |
| TOTAL | 20.0 |

Preparation:

The active substance is mixed with lactose for inhalation. The mixture is packed into capsules in a capsule-making machine (weight of the empty capsule approx. 50 mg). Weight of capsule: 70.0 mg; size of capsule: 3.

EXAMPLE 18
Solution for Inhalation for Hand-Held
Nebulisers Containing 2.5 mg of Active Substance

| Component | Amount per spray |
|---|---|
| active substance | 2.500 mg |
| benzalkonium chloride | 0.001 mg |
| 1N hydrochloric acid | q.s. |
| ethanol/water (50/50) | ad 15.000 mg |

Preparation:

The active substance and benzalkonium chloride are dissolved in ethanol/water (50/50). The pH of the solution is adjusted with IN hydrochloric acid. The resulting solution is filtered and transferred into suitable containers for use in hand-held nebulizers (cartridges). Contents of the container: 4.5 g.

We claim:

1. A compound of formula (I) 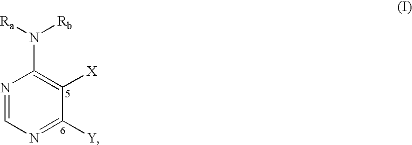
wherein:
Rₐ is a hydrogen atom or a C₁₋₄-alkyl group;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, bromine, or iodine atom,
(ii) a C₁₋₄-alkyl, hydroxy, C₁₋₄-alkoxy, C₃₋₆-cycloalkyl, C₄₋₆-cycloalkoxy, C₂₋₅-alkenyl, or C₂₋₅-alkynyl group,
(iii) an aryl, aryloxy, arylmethyl, or arylmethoxy group,
(iv) a C₃₋₅-alkenyloxy or C₃₋₅-alkynyloxy group, wherein the unsaturated moiety is not linked to the oxygen atom,
(v) a C₁₋₄-alkylsulfenyl, C₁₋₄-alkylsulfinyl, C₁₋₄-alkylsulfonyl, C₁₋₄-alkylsulfonyloxy, trifluoromethylsulfenyl, trifluoromethylsulfinyl, or trifluoromethylsulfonyl group,
(vi) a methyl or methoxy group substituted by 1 to 3 fluorine atoms,
(vii) an ethyl or ethoxy group substituted by 1 to 5 fluorine atoms, or
(viii) a cyano or nitro group or an amino group optionally substituted by one or two C₁₋₄-alkyl groups, wherein the substituents are identical or different, or
R₁ together with R₂, if they are bound to adjacent carbon atoms, are a —CH═CH—CH═CH, —CH═CH—NH, or —CH═N—NH group, and
R₃ is a hydrogen, fluorine, chlorine, or bromine atom, or a C₁₋₄-alkyl, trifluoromethyl, or C₁₋₄-alkoxy group;
X and Y together are a —N═C(—A—B)—CH═CH—, —CH═N—C(—A—B)═CH—, —CH═C(—A—B)—N═CH—, —CH═CH—C(—A—B)═N—, —N═C(—A—B)—N═CH—, or —CH═N—C(—A—B)═N— bridge, wherein the left-hand end of these bridges is linked to position 5 and the right-hand end of these bridges is linked to position 6 of the pyrimidine ring of the compound of formula (I), and
A is a group consisting of:
(a) an —O—C₁₋₈-alkylene, —O—C₄₋₇-cycloalkylene, —O—C₁₋₃-alkylene-C₃₋₇-cycloalkylene, —O—C₄₋₇-cycloalkylene-C₁₋₃-alkylene, or —O—C₁₋₃-alkylene-C₃₋₇-cycloalkylene-C₁₋₃-alkylene group, wherein the oxygen atom thereof in each case is linked to the bicyclic heteroaromatic moiety of formula (I),
(b) an —NR₄—C₁₋₈-alkylene, —NR₄—C₃₋₇-cycloalkylene, —NR₄—C₁₋₃-alkylene-C₃₋₇-cycloalkylene, —NR₄—C₃₋₇-cycloalkylene-C₁₋₃-alkylene, or —NR₄—C₁₋₃-alkylene-C₃₋₇-cycloalkylene-C₁₋₃-alkylene group, wherein the —NR₄-moiety thereof in each case is linked to the bicyclic heteroaromatic moiety of formula (I),
(c) an oxygen atom linked to a carbon atom of the group B,
(d) an —NR₄—C₄₋₇-cycloalkylene-NH—SO₂—C₁₋₄-alkylene or —NR₄—C₄₋₇-cycloalkylene-N(C₁₋₄-alkyl)-SO₂—C₁₋₄-alkylene group, wherein the —NR₄-moiety thereof in each case is linked to the bicyclic heteroaromatic moiety of formula (I),
(e) an —NR₄ group linked to a carbon atom of the group B,
(f) an azetidinylene, pyrrolidinylene, piperidinylene, or hexahydroazepinylene group optionally substituted by one or two methyl groups, wherein in each case the cyclic nitrogen atom thereof is linked to the bicyclic heteroaromatic moiety of formula (I),
(g) an azetidinylene-C₁₋₃-alkylene, pyrrolidinylene-C₁₋₃-alkylene, piperidinylene-C₁₋₃-alkylene, or hexahydroazepinylene-C₁₋₃-alkylene group, wherein in each case the cyclic nitrogen atom thereof is linked to the bicyclic heteroaromatic moiety of formula (I),
(h) a 1,4-piperazinylene or 1,4-homopiperazinylene group, each linked to a carbon atom of the group B,
(i) a 1,4-piperazinylene-C₁₋₃-alkylene or 1,4-homopiperazinylene-C₁₋₃-alkylene group, wherein in each case the cyclic nitrogen atom thereof is linked to the bicyclic heteroaromatic moiety of formula (I),
(j) an —NR₄-azetidinylene, —NR₄-pyrrolidinylene, —NR₄-piperidinylene, or —NR₄-hexahydroazepinylene group, wherein the —NR₄— moiety thereof is linked in each case to the bicyclic heteroaromatic moiety of formula (I) and in each case the cyclic nitrogen atom thereof is linked to a carbon atom of the group B,
(k) an —NR₄-azetidinylene-C₁₋₃-alkylene, —NR₄-pyrrolidinylene-C₁₋₃-alkylene, —NR₄-piperidinylene-C₁₋₃-alkylene, or —NR₄-hexahydroazepinylene-C₁₋₃-alkylene group, wherein in each case the —NR₄— moiety thereof is linked to the bicyclic heteroaromatic moiety of formula (I) and the cyclic nitrogen atom thereof is in each case linked to the alkylene moiety,
(l) an —NR₄—C₃₋₇-cycloalkylenecarbonyl group, wherein the —NR₄— moiety is linked to the bicyclic heteroaromatic moiety of compound (I) and the carbonyl group is linked to a nitrogen atom of the group B,
(m) an —NR₄—C₃₋₇-cycloalkylenecarbonylamino group, wherein the —NR₄— moiety is linked to the bicyclic heteroaromatic moiety of formula (I) and the nitrogen atom of the carbonylamino moiety, which is additionally optionally substituted by a C₁₋₄-alkyl group, is linked to a carbon atom of the group B,
(n) an —NR₄—C₃₋₇-cycloalkylenecarbonylamino-C₁₋₃-alkylene group, wherein the —NR₄— moiety is linked to the bicyclic heteroaromatic moiety of formula (I) and the nitrogen atom of the carbonylamino moiety is additionally optionally substituted by a C₁₋₄-alkyl group,
(o) an azetidinylenecarbonyl, pyrrolidinylenecarbonyl, piperidinylenecarbonyl, or hexahydroazepinylenecarbonyl group, wherein in each case the cyclic nitrogen atom thereof is linked to the bicyclic heteroaromatic moiety of compound (I) and the carbonyl group in each case is linked to a nitrogen atom of the group B,
(p) an azetidinylenecarbonylamino, pyrrolidinylenecarbonylamino, piperidinylenecarbonylamino, or hexahydroazepinylenecarbonylamino group, wherein in each case the cyclic nitrogen atom thereof is linked to the bicyclic heteroaromatic moiety of compound (I) and the nitrogen atom of the carbonylamino moiety, which is additionally optionally substituted by a C₁₋₄-alkyl group, is linked to a carbon atom of the group B, and
(q) an azetidinylenecarbonylamino-C₁₋₃-alkylene, pyrrolidinylenecarbonylamino-C₁₋₃-alkylene, piperidinylenecarbonylamino-C₁₋₃-alkylene, or hexahydroazepinylenecarbonylamino-C₁₋₃-alkylene group, wherein in each case the cyclic nitrogen atom thereof is linked to the bicyclic heteroaromatic moiety of compound (I) and the nitrogen atom of the carbonylamino moiety is optionally additionally substituted by a C₁₋₄-alkyl group,
B is a group consisting of:
(a) an R₆O—CO-alkylene-NR₅, (R₇O—PO—OR₈)-alkylene-NR₅, or (R₇O—PO—R)-alkylene-NR₅ group wherein in each case the alkylene moiety, which is straight-chained and contains 1 to 6 carbon atoms, is additionally optionally substituted by one or two C₁₋₂-alkyl groups or by an R₆O—CO or R₆O—CO—C₁₋₂-alkyl group,
(b) a 4- to 7-membered alkyleneimino group substituted by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group,
(c) a piperazino or homopiperazino group substituted in the 4 position by R₁₀ and additionally at a cyclic carbon atom by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR)—C₁₋₄-alkyl, or (R₇O—PO—R₉)—C₁₋₄-alkyl group;
(d) a piperazino or homopiperazino group substituted in each case in the 4 position by an R₆O—CO—C₁₋₄-alkyl, bis(R₆O—CO)—C₁₋₄-alkyl, (R₇O—PO—OR)—C₁₋₄-alkyl, or (R₇O—PO—R₉)—C₁₋₄-alkyl group,
(e) a pyrrolidinyl, piperidinyl, or hexahydroazepinyl group substituted in the 1 position by R₁₀, wherein the 5- to 7-membered rings are each additionally substituted at a carbon atom by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR₈)—C₁₋₄-alkyl, or (R₇O—PO—R₉)-C₁₋₄-alkyl group,
(f) a pyrrolidinyl, piperidinyl, or hexahydroazepinyl group substituted in the 1 position by an R₆O—CO—C₁₋₄-alkyl, bis(R₆O—CO)—C₁₋₄-alkyl, (R₇O—PO—OR)—C₁₋₄-alkyl, or (R₇O—PO—R₉)—C₁₋₄-alkyl group,
(g) a 2-oxomorpholino group optionally substituted by one or two methyl groups,
(h) a 2-oxomorpholinyl group substituted in the 4 position by a hydrogen atom, or by a C₁₋₄-alkyl, R₆O—CO—C₁₋₄-alkyl, (R₇O—PO—OR₈)—C₁₋₄-alkyl, or (R₇O—PO—R₉)—C₁₋₄-alkyl group, wherein the 2-oxomorpholinyl group thereof in each case is linked to a carbon atom of the group A,
(i) a C₅₋₇-cycloalkyl group substituted by an amino, C₁₋₄-alkylamino, or di-(C₁₋₄-alkyl)-amino group and by an R₆O—CO group,
(j) a C₅₋₇-cycloalkyl group wherein a methylene group is replaced by an R₆O—CO—C₁₋₄-alkyleneimino, [bis(R₆O—CO)—C₁₋₄-alkylene]imino, (R₇O—PO—OR₈)-C₁₋₄-alkyleneimino, or (R₇O—PO—OR₉)—C₁₋₄-alkyleneimino group and in each case two hydrogen atoms in the cycloalkyl moiety are replaced by a straight-chained alkylene bridge, this bridge containing 2 to 6 carbon atoms, if the two hydrogen atoms are located at the same carbon atom, or contains 1 to 5 carbon atoms if the two hydrogen atoms are located at adjacent carbon atoms, or contains 2 to 4 carbon atoms, if the two hydrogen atoms are located at carbon atoms which are separated by one atom, or
A together with B are a group consisting of:
(a) 1-azetidinyl group wherein the two hydrogen atoms of a methylene group are replaced by a straight-chained C₄₋₆-alkylene bridge, wherein in each case a methylene group in the C₄₋₆-alkylene bridge is replaced by an R₆O—CO—C₁₋₄-alkyleneimino, [bis(R₆O—CO)—C₁₋₄-alkylene]imino, (R₇O—PO—OR₈)—C₁₋₄-alkyleneimino, or (R₇O—PO—R₉)—C₁₋₄-alkyleneimino group,
(b) a 1-pyrrolidinyl, 1-piperidinyl, or 1-azacyclohept-1-yl group wherein the two hydrogen atoms of a methylene group are replaced by a straight-chained C₃₋₆-alkylene bridge, wherein in each case a methylene group in the C₃₋₆-alkylene bridge is replaced by an R₆O—CO—C₁₋₄-alkyleneimino, [bis(R₆O—CO)—C₁₋₄-alkylene]imino, (R₇O—PO—OR₈)—C₁₋₄-alkyleneimino, or (R₇O—PO—R₉)—C₁₋₄-alkyleneimino group,
(c) a pyrrolidino, piperidino, or hexahydroazepino group which are substituted in each case by an amino, C₁₋₄-alkylamino, or di-(C₁₋₄-alkyl)-amino group and by an R₆O—CO group,
(d) a piperazino or homopiperazino group substituted in the 4 position by Rio and additionally at a cyclic carbon atom by an R₆O—CO, (R₇O—PO—OR₈), (R₇O—PO—R₉), R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group,
(d) a piperazino or homopiperazino group substituted in each case in the 4 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group, and
(e) a 2-oxomorpholino group optionally substituted by one or two methyl groups;
R₄ is a hydrogen atom or a C₁₄-alkyl group;
R₅ is a hydrogen atom,
a C₁₋₄-alkyl group optionally substituted by a hydroxy, C₁₋₄-alkoxy, carboxy, R₆O—CO, (R₇O—PO—OR₈), (R₇O—PO—R₉), amino, C₁₋₄-alkylamino, or di-(C₁₋₄-alkyl)-amino group, or by a 4- to 7-membered alkyleneimino group, wherein in the 6- to 7-membered alkyleneimino groups in each case, a methylene group in the 4 position is optionally replaced by an oxygen or sulfur atom, or by a sulfinyl, sulfonyl, imino, or N—(C₁₋₄-alkyl)-imino group, or
a C₃₋₇-cycloalkyl or C₃₋₇-cycloalkyl-C₁₋₃-alkyl group;
R₆, R₇, and R₈, which are identical or different, in each case are a hydrogen atom,
a C₁₋₈-alkyl group optionally substituted by a hydroxy, C₁₋₄-alkoxy, amino, C₁₋₄-alkylamino, or di-(C₁₋₄-alkyl)-amino group or by a 4- to 7-membered alkyleneimino group, wherein in the 6- to 7-membered alkyleneimino group thereof in each case a methylene group in the 4 position is optionally replaced by an oxygen or sulfur atom, or by a sulfinyl, sulfonyl, imino, or N—(C₁₋₄-alkyl)-imino group,
a C₄₋₇-cycloalkyl group optionally substituted by one or two methyl groups, a C₃₋₅-alkenyl or C₃₋₅-alkynyl group, wherein the unsaturated moiety is not linked to the oxygen atom, or
a C₃₋₇-cycloalkyl-C₁₋₄-alkyl, aryl, aryl-C₁₋₄-alkyl, or RₑCO—O—(R_{c}CR_{d}) group;
R₉ is a C₁₋₄-alkyl, aryl, or aryl-C₁₋₄-alkyl group;
R₁₀ is a hydrogen atom, a C₁₋₄-alkyl, formyl, C₁₋₄-alkylcarbonyl, or C₁₋₄-alkylsulfonyl group;
R₁₁ is a cyano, carboxy, C₁₋₄-alkoxycarbonyl, aminocarbonyl, C₁₋₄-alkylaminocarbonyl, di-(C₁₋₄-alkyl)-aminocarbonyl, C₁₋₄-alkylsulfenyl, C₁₋₄-alkylsulfinyl, C₁₋₄-alkylsulfonyl, hydroxy, C₁₋₄-alkylsulfonyloxy, trifluoromethyloxy, nitro, amino, C₁₋₄-alkylamino, di-(C₁₋₄-alkyl)-amino, C₁₋₄-alkylcarbonylamino, N—(C₁₋₄-alkyl)—C₁₋₄-alkylcarbonylamino, C₁₋₄-alkylsulfonylamino, N—(C₁₋₄-alkyl)—C₁₋₄-alkylsulfonylamino, aminosulfonyl, C₁₋₄-alkylaminosulfonyl, or di-(C₁₋₄-alkyl)-aminosulfonyl group or a carbonyl group substituted by a 5- to 7-membered alkyleneimino group, wherein in the 6- to 7-membered alkyleneimino groups thereof in each case a methylene group in the 4 position is optionally replaced by an oxygen or sulfur atom, or by a sulfinyl, sulfonyl, imino, or N—(C₁₋₄-alkyl)-imino group;
R₁₂ is a fluorine, chlorine, bromine, or iodine atom, or a C₁₋₄-alkyl, trifluoromethyl, or C₁₋₄-alkoxy group, or two R₁₂ groups, if they are bound to adjacent carbon atoms, together are a C₃₋₅-alkylene, methylenedioxy, or 1,3-butadien-1,4-ylene group;
R_{c} and R_{d}, which are identical or different, in each case are a hydrogen atom or a C₁₋₄-alkyl group; and
Rₑ is a C₁₋₄-alkyl, C₃₋₇-cycloalkyl, C₁₋₄-alkoxy, or C₅₋₇-cycloalkoxy group,
wherein the aryl moieties of the abovementioned groups are identical or different phenyl groups optionally monosubstituted by R₁₁, mono-, di-, or trisubstituted by R₁₂, or monosubstituted by R₁₁ and additionally mono- or disubstituted by R₁₂, wherein the substituents are identical or different,
or a tautomer, stereoisomer, or salt thereof.

2. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, or bromine atom,
(ii) a methyl, trifluoromethyl, ethynyl, or amino group, or
(iii) a phenyl, phenoxy, benzyl, or benzyloxy group, or
R₁ together with R₂, if they are bound to adjacent carbon atoms, are a —CH═CH—NH or —CH═N—NH group, and
R₃ is a hydrogen, fluorine, chlorine, or bromine atom;
X and Y together are a —N═C(—A—B)—CH═CH—, —CH═N—C(—A—B)═CH—, —CH═C(—A—B)—N═CH—, —CH═CH—C(—A—B)═N—, —N═C(—A—B)—N═CH—, or —CH—N—C(—A—B)═N— bridge, wherein the left-hand end of these bridges is linked to position 5 and the right-hand end of these bridges is linked to position 6 of the pyrimidine ring of the compound of formula (I),
A is a group consisting of:
(a) an —NR₄—C₁₋₄-alkylene, —NR₄-cyclohexylene, —NR₄-cyclohexylene-NH—SO₂-C₁₋₃-alkylene, —NR₄—C₁₋₃-alkylene-cyclohexylene, —NR₄-cyclohexylene-C₁₋₃-alkylene, or —NR₄—C₁₋₃-alkylene-cyclohexylene-C₁₋₃-alkylene group, wherein the —NR₄— moiety thereof in each case is linked to the bicyclic heteroaromatic moiety of compound (I),
(b) an —NR₄ group linked to a carbon atom of the group B,
(c) a pyrrolidinylene or piperidinylene group optionally substituted by one or two methyl groups, wherein in each case the cyclic nitrogen atom thereof is linked to the bicyclic heteroaromatic moiety of compound (I),
(d) a piperidinylene-C₁₋₃-alkylene group, wherein the cyclic nitrogen atom thereof is linked to the bicyclic heteroaromatic moiety of compound (I),
(e) a 1,4-piperazinylene or 1,4-homopiperazinylene group, each of these groups linked to a carbon atom of the group B,
(f) a 1,4-piperazinylene-C₁₋₂-alkylene or 1,4-homopiperazinylene-C₁₋₂-alkylene group, wherein in each case the cyclic nitrogen atom thereof is linked to the bicyclic heteroaromatic moiety of compound (I),
(g) an —NR₄-piperidinylene group, wherein the —NR₄— moiety thereof is linked to the bicyclic heteroaromatic moiety of compound
(i) and the cyclic nitrogen atom thereof is linked to a carbon atom of the group B,
(h) an —NR₄-piperidinylene-C₁₋₂-alkylene group, wherein the —NR₄— moiety thereof is linked to the bicyclic heteroaromatic moiety of compound (I) and the cyclic nitrogen atom thereof is linked to the alkylene moiety,
(i) an —NR₄-cyclohexylenecarbonyl group, wherein the —NR₄— moiety is linked to the bicyclic heteroaromatic moiety of compound (I) and the carbonyl group is linked to a nitrogen atom of the group B,
(j) an —NR₄-cyclohexylenecarbonylamino group, wherein the —NR₄— moiety is linked to the bicyclic heteroaromatic moiety of compound (I) and the nitrogen atom of the carbonylamino moiety is linked to a carbon atom of the group B,
(k) an —NR₄-cyclohexylenecarbonylamino-C₁₋₂-alkylene group, wherein the —NR₄— moiety is linked to the bicyclic heteroaromatic moiety of compound (I),
(l) a piperidinylenecarbonyl group, wherein the cyclic nitrogen atom thereof is linked to the bicyclic heteroaromatic moiety of compound (I) and the carbonyl group is linked to a nitrogen atom of the group B,
(m) a piperidinylenecarbonylamino group, wherein the cyclic nitrogen atom thereof is linked to the bicyclic heteroaromatic moiety of compound (I) and the nitrogen atom of the carbonylamino moiety is linked to a carbon atom of the group B, and
(n) a piperidinylenecarbonylamino-C₁₋₂-alkylene group, wherein the cyclic nitrogen atom thereof is linked to the bicyclic heteroaromatic moiety of compound (I),
B is a group consisting of:
(a) an R₆—CO-alkylene-NR₅, (R₇O—PO—OR₈)-alkylene-NR₅, or (R₇O—PO—R₉)-alkylene-NR₅ group wherein in each case the alkylene moiety, which is straight-chained and contains 1 to 4 carbon atoms, is additionally optionally substituted by one or two C₁₋₂-alkyl groups or by an R₆O—CO or R₆O—CO—C₁₋₂-alkyl group,
(b) a pyrrolidino or piperidino group substituted in each case by an R₆O—CO or R₆O—CO—C₁₋₄-alkyl group,
(c) a piperazino or homopiperazino group substituted in each case in the 4 position by R₁₀ and is additionally substituted at a cyclic carbon atom by an R₆O—CO or R₆O—CO—C₁₋₄-alkyl group,
(d) a piperazino or homopiperazino group substituted in each case in the 4 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group,
(e) a pyrrolidinyl or piperidinyl group substituted in the 1 position by R₁₀, which is additionally substituted in each case at a carbon atom by an R₆O—CO or R₆O—CO—C₁₋₄-alkyl group,
(f) a pyrrolidinyl, piperidinyl, or hexahydroazepinyl group substituted in the 1 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group,
(g) a 2-oxomorpholino group optionally substituted by one or two methyl groups,
(h) a 2-oxomorpholinyl group substituted in the 4 position by a hydrogen atom, or by a methyl, ethyl, or R₆O—CO—C₁₋₄-alkyl group, wherein the 2-oxomorpholinyl group thereof in each case are linked to a carbon atom of the group A,
(i) a C₅₋₆-cycloalkyl group substituted by an amino, C₁₋₂-alkylamino, or di-(C₁₋₂-alkyl)-amino group and by an R₆O—CO group, or
A together with B are a group consisting of:
(a) a 1-pyrrolidinyl or 1-piperidinyl group wherein the two hydrogen atoms of a methylene group are replaced by a straight-chained C₄₋₅-alkylene bridge, wherein in each case a methylene group in the C₄₋₅-alkylene bridge is replaced by an R₆O—CO—C₁₋₄-alkyleneimino group,
(b) a pyrrolidino or piperidino group substituted in each case by an amino, C₁₋₂-alkylamino, or di-(C₁₋₂-alkyl)-amino group and by an R₆O—CO group,
(c) a piperazino or homopiperazino group substituted in the 4 position by R₁₀ and additionally at a cyclic carbon atom by an R₆O—CO or R₆O—CO—C₁₋₄-alkyl group,
(d) a piperazino or homopiperazino group substituted in each case in the 4 position by an R₆O—CO—C₁₋₄-alkyl, bis(R₆O—CO)—C₁₋₄-alkyl, (R₇O—PO—OR₈)—C₁₋₄-alkyl, or (R₇O—PO—R₉)—C₁₋₄-alkyl group, and
(e) a 2-oxomorpholino group optionally substituted by one or two methyl groups;
R₄ is a hydrogen atom or a methyl group;
R₅ is a hydrogen atom or a C₁₋₄-alkyl group optionally substituted by an R₆O—CO group;
R₆, R₇, and R₈, which are identical or different, in each case are a hydrogen atom,
a C₁₋₈-alkyl group,
a cyclopentyl, cyclopentylmethyl, cyclohexyl, or cyclohexylmethyl group, or
a phenyl group optionally substituted by one or two methyl groups, a 5-indanyl group or a benzyl group optionally substituted in the phenyl moiety by one or two methyl groups;
R₉ is a methyl or ethyl group; and
R₁₀ is a hydrogen atom, or a methyl or ethyl group,
or a tautomer, stereoisomer, or salt thereof.

3. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are:
(i) a hydrogen, fluorine, chlorine, or bromine atom, or
(ii) a methyl, trifluoromethyl, ethynyl, or amino group, or
R₁ together with R₂, if they are bound to adjacent carbon atoms, are a —CH═CH—NH group and
R₃ is a hydrogen, fluorine, chlorine, or bromine atom;
X and Y together are a —N═C(—A—B)—CH═CH—, —CH═N—C(—A—B)═CH—, —CH═C(—A—B)—N═CH—, —CH═CH—C(—A—B)═N—, —N═C(—A—B)—N═CH—, or —CH═N—C(—A—B)═N— bridge, wherein the left-hand end of these bridges is linked to position 5 and the right-hand end of these bridges is linked to position 6 of the pyrimidine ring, and
A is a group consisting of:
(a) an —NR₄-C₁₋₄-alkylene, —NR₄-cyclohexylene, —NR₄-cyclohexylene-NH—SO₂-C₁₋₃-alkylene, —NR₄-methylene-cyclohexylene, —NR₄-cyclohexylene-methylene, or —NR₄-methylene-cyclohexylene-methylene group, wherein the —NR₄— moiety thereof in each case is linked to the bicyclic heteroaromatic moiety of compound (I),
(b) an —NR₄ group linked to a carbon atom of the group B,
(c) a pyrrolidinylene or piperidinylene group optionally substituted by one or two methyl groups, wherein in each case the cyclic nitrogen atom thereof is linked to the bicyclic heteroaromatic moiety of compound (I),
(d) a piperidinylene-C₁₋₂-alkylene group, wherein the cyclic nitrogen atom thereof is linked to the bicyclic heteroaromatic moiety of compound (I),
(e) a 1,4-piperazinylene group linked to a carbon atom of the group B,
(f) a 1,4-piperazinylene-C₁₋₂-alkylene group, wherein the cyclic nitrogen atom thereof is linked to the bicyclic heteroaromatic moiety of compound (I),
(g) an —NR₄-piperidinylene group, wherein the —NR₄— moiety thereof is linked to the bicyclic heteroaromatic moiety of compound (I) and the cyclic nitrogen atom thereof is linked to a carbon atom of the group B,
(h) an —NR₄-cyclohexylenecarbonylamino group, wherein the —NR₄— moiety is linked to the bicyclic heteroaromatic moiety of compound (I) and the nitrogen atom of the carbonylamino moiety is linked to a carbon atom of the group B, and
(i) an —NR₄-cyclohexylenecarbonylamino-C₁₋₂-alkylene group, wherein the —NR₄— moiety is linked to the bicyclic heteroaromatic moiety of compound (I),
B is a group consisting of:
(a) an R₆O—CO-alkylene-NR₅, (R₇O—PO—OR₈)-alkylene-NR₅, or (R₇O—PO—R₉)-alkylene-NR₅ group wherein in each case the alkylene moiety is straight-chained and contains 1 to 4 carbon atoms,
(b) a pyrrolidino or piperidino group substituted in each case by an R₆O—CO or R₆O—CO—C₁₋₂-alkyl group,
(c) a piperazino group substituted in the 4 position by an R₆O—CO—C₁₋₃-alkyl or (R₇O—PO—R₉)—C₁₋₃-alkyl group,
(d) a pyrrolidinyl, piperidinyl, or hexahydroazepinyl group substituted in the 1 position by an R₆O—CO—C₁₋₄-alkyl, bis(R₆O—CO)—C₁₋₄-alkyl, (R₇O—PO—OR₈)-C₁₋₄-alkyl, or (R₇O—PO—R₉)-C₁₋₄-alkyl group, and
(e) a 2-oxomorpholino group optionally substituted by one or two methyl groups, or
A together with B are a group consisting of:
(a) a 1-pyrrolidinyl or 1-piperidinyl group wherein the two hydrogen atoms of a methylene group are replaced by a straight-chained C₄₋₅-alkylene bridge, wherein in each case a methylene group in the C₄₋₅-alkylene bridge is replaced by an R₆O—CO—C₁₋₂-alkyleneimino group,
(d) a piperidino group substituted by an amino group and by an R₆—CO group,
(e) a piperazino group substituted in the 4 position by an R₆O—CO—C₁₋₄-alkyl group, and
(f) a 2-oxomorpholino group optionally substituted by one or two methyl groups;
R₄ is a hydrogen atom or a methyl group;
R₅ is a hydrogen atom or a C₁₋₂-alkyl group optionally substituted by an R₆O—CO group;
R₆, R₇, and R₈, which are identical or different, in each case are a hydrogen atom,
a C₁₋₈-alkyl group,
a cyclopentyl, cyclohexyl, cyclopentylmethyl, or cyclohexylmethyl group, or
a phenyl group optionally substituted by one or two methyl groups, a 5-indanyl group or a benzyl group optionally substituted in the phenyl moiety by one or two methyl groups; and
R₉ is a methyl or ethyl group,
or a tautomer, stereoisomer, or salt thereof.

4. The compound of formula (I) according to claim 1, wherein X and Y together are an —N═C(—A—B)—N═CH— bridge.

5. The compound of formula (I) according to claim 1, wherein:
Rₐ is a hydrogen atom;
R_{b} is a phenyl, benzyl, or 1-phenylethyl group wherein the phenyl nucleus is substituted in each case by R₁, R₂, and R₃, wherein:
R₁ and R₂, which are identical or different, each are a hydrogen, fluorine, chlorine, or bromine atom, or a methyl or amino group, or
R₁ together with R₂, if they are bound to adjacent carbon atoms, are an —CH═CH—NH group, and
R₃ is a hydrogen, fluorine, chlorine, or bromine atom;
X and Y together are an —N═C(—A—B)—N═CH— bridge, wherein the left-hand end of this bridge is linked to position 5 and the right-hand end of this bridge is linked to position 6 of the pyrimidine ring,
A is a group consisting of:
(a) an —NR₄—C₁₋₃-alkylene, —NR₄-cyclohexylene, or —NR₄-cyclohexylene-NH—SO₂-ethylene group, wherein the —NR₄— moiety thereof in each case is linked to the bicyclic heteroaromatic moiety of compound (I),
(b) an —NR₄ group linked to a carbon atom of the group B,
(c) an optionally methyl-substituted pyrrolidinylene or piperidinylene group, wherein in each case the cyclic nitrogen atom thereof is linked to the bicyclic heteroaromatic moiety of compound (I),
(d) a piperidinylenemethylene group, wherein the cyclic nitrogen atom thereof is linked to the bicyclic heteroaromatic moiety of compound (I), and
(e) a 1,4-piperazinylene group linked to a carbon atom of the group B,
B is a group consisting of:
(a) an R₆O—CO-alkylene-NR₅ group wherein the alkylene moiety is straight-chained and contains 1 to 4 carbon atoms,
(b) a pyrrolidino or piperidino group substituted in each case by an R₆O—CO or R₆O—CO—C₁₋₂-alkyl group,
(c) a piperazino group substituted in the 4 position by an R₆O—CO-methyl or (R₇O—PO—OR₈)-methyl group,
(d) a piperidinyl group substituted in the 1 position by an R₆O—CO—C₁₋₄-alkyl, bis(R₆O—CO)—C₁₋₄-alkyl or (R₇O—PO—R₉)-methyl group, and
(e) a 2-oxomorpholino group,
A together with B are a group consisting of:
(a) a piperidino group substituted by an amino group and by an R₆O—CO group, and
(b) a piperazino group substituted in the 4 position by an R₆O—CO—C₁₋₂-alkyl group,
R₄ is a hydrogen atom or a methyl group;
R₅ is a hydrogen atom, or a C₁₋₂-alkyl group optionally substituted by an R₆O—CO group;
R₆ is a hydrogen atom, or a C₁₋₄-alkyl group or a cyclohexyl group;
R₇ and R₈ in each case is a methyl or ethyl group; and
R₉ is a methyl or ethyl group,
or a tautomer, stereoisomer, or salt thereof.

6. A compound selected from the group consisting of:
(1) 4-[(3-chloro-4-fluorophenyl)amino]-6-({1-[(methoxycarbonyl)methyl]piperidin-4-yl}amino)pyrimido[5,4-d]pyrimidine;
(2) 4-[(3-chloro-4-fluorophenyl)amino]-6-[(trans-4-{N-[(methoxycarbonyl)methyl]-N-methylamino}cyclohex-1-yl)amino]pyrimido[5 ,4-d]pyrimidine;
(3) 4-[(3-chloro-4-fluorophenyl)amino]-6-[4-({N-[(methoxycarbonyl)methyl]-N-methylamino}methyl)piperidin-1-yl]pyrimido[5,4-d]pyrimidine;
(4) 4-[(3-bromophenyl)amino]-6-({1-[(methoxycarbonyl)methyl]piperidin-4-yl}amino)pyrimido[5,4-d]pyrimidine;
(5) 4-[(3-chlorophenyl)amino]-6-({1-[(methoxycarbonyl)methyl]piperidin-4-yl}amino)pyrimido[5,4-d]pyrimidine;
(6) 4-[(3-methylphenyl)amino]-6-({1-[(methoxycarbonyl)methyl]piperidin-4-yl}amino)pyrimido[5,4-d]pyrimidine;
(7) 4-[(4-amino-3,5-dichlorophenyl)amino]-6-({1-[(methoxycarbonyl)methyl]piperidin-4-yl}amino)pyrimido[5,4-d]pyrimidine;
(8) 4-[(4-amino-3,5-dibromophenyl)amino]-6-({1-[(methoxycarbonyl)methyl]piperidin-4-yl}amino)pyrimido[5,4-d]pyrimidine;
(9) 4-[(indol-5-yl)amino]-6-({1-[(methoxycarbonyl)methyl]piperidin-4-yl}amino)pyrimido [5,4-d]pyrimidine;
(10) 4-[(3-chloro-4-fluorophenyl)amino]-6-[(trans-4-{N,N-bis-[(ethoxycarbonyl)methyl]amino}cyclohex-1-yl)amino]pyrimido [5,4-d]pyrimidine;
(11) 4-[(3-chloro-4-fluorophenyl)amino]-6-[(trans-4-{N,N-bis-[(methoxycarbonyl)methyl]amino}cyclohex-1-yl)amino]pyrimido [5,4-d]pyrimidine;
(12) 4-[(3-chloro-4-fluorophenyl)amino]-6-[(trans-4-{[(methoxy-carbonyl)methyl]amino}cyclohex-1-yl)amino]pyrimido[5,4-d]pyrimidine;
(13) 4-[(3-chloro-4-fluorophenyl)amino]-6-[N-(trans-4-{N′,N′-bis[(methoxycarbonyl)methyl]amino}cyclohex-1-yl)-N-methylamino]pyrimido [5,4-d]pyrimidine;
(14) 4-[(3-bromophenyl)amino]-6-(1-[1,1-bis(methoxycarbonyl)methyl]piperidin-4-yl} amino)pyrimido[5,4-d]pyrimidine;
(15) 4-[(3-bromophenyl)amino]-6-({1-[(methoxycarbonyl)methyl]piperidin-3-yl}amino)pyrimido[5,4-d]pyrimidine;
(16) 4-[(3-chloro-4-fluorophenyl)amino]-6-(1-[1,1-bis(methoxycarbonyl)methyl]piperidin-4-yl}amino)pyrimido[5,4-d]pyrimidine;
(17) 4-[(3-bromophenyl)amino]-6-({1-[(diethoxyphosphoryl)methyl]piperidin-4-yl}amino)pyrimido[5,4-d]pyrimidine;
(18) 4-[(3-bromophenyl)amino]-6-[(1-{[(ethoxy)(methyl)phosphoryl]methyl}piperidin-4-yl)amino]pyrimido[5,4-d]pyrimidine; and
(19) 4-[(3-chloro-4-fluorophenyl)amino]-6-{[trans-4-(2-oxomorpholin-4-yl)cyclohex-1-yl]amino}pyrimido[5,4-d]pyrimidine,
and a tautomer, stereoisomer, or salt thereof.

7. The compound according to one of claims 1 to 6, wherein the compound is a physiologically acceptable salt.

8. A pharmaceutical composition comprising an effective amount of a compound of formula (I) according to one of claims 1 to 7 and an inert carrier or diluent.

9. A method for treatment or prophylaxis of benign or malignant tumors, diseases of the airways and lungs, polyps, diseases of the gastrointestinal tract, bile duct, gall bladder, kidneys, and skin, in a host in need of such treatment or prophylaxis, which method comprises administering the host an effective amount of a compound according to one of claims 1 to 7.
